(19) United States
(12) Patent Application Publication
(10) Pub. No.: US 2026/0306388 A1
(43) Pub. Date: Oct. 8, 2026

(54) COMPOSITION COMPRISING BIOSURFACTANT AND MANGANESE

(71) Applicant: Evonik Operations GmbH, Essen (DE)

(72) Inventors: Eric Hüfner, Hainburg (DE); Juliane Merz, Dortmund (DE); Wiebke Buck, Gelnhausen (DE); Jan Hellriegel, Mainz (DE)

(73) Assignee: Evonik Operations GmbH, Essen (DE)

(21) Appl. No.: 19/491,438

(22) PCT Filed: Jun. 7, 2024
(86) PCT No.: PCT/EP2024/065749

§ 371 Date: Dec. 9, 2025

(30) Foreign Application Priority Data
Jun. 22, 2023 (EP) 23180910.4

(51) Int. Cl. A61K 8/68; A61K 8/19; A61K 8/23; A61Q 5/02; A61Q 19/10; C11D 1/36; C11D 1/66; C11D 3/04; C11D 3/12; C11D 3/48

(52) U.S. Cl. CPC A61K 8/68; A61K 8/19; A61K 8/23; A61Q 5/02; A61Q 19/10; C11D 1/36; C11D 1/662; C11D 3/046; C11D 3/1213; C11D 3/48; A61K 2800/524

(57) ABSTRACT

A composition has at least one biosurfactant and manganese. A method stabilizes the colour of a composition having the at least one biosurfactant. Manganese can be used to stabilize the colour of a composition having the at least one biosurfactant.

### FIELD OF THE INVENTION

The invention relates to compositions comprising biosurfactant and manganese.

It further relates to a method to stabilize the colour of a composition comprising biosurfactant and to the use of manganese to stabilize the colour of a composition comprising biosurfactant.

### PRIOR ART

Surfactants are used in many different compositions and applications. Due to the need of more sustainability biosurfactants are replacing classical surfactants, which are frequently based on petrochemicals or non-renewable raw materials.

Biosurfactants, being glycolipids produced by fermentation, are difficult to purify and/or concentrate in a colourless way. They also have the disadvantage, that they suffer from colour instability and tend to become darker and or brownish during storage.

While not necessarily a major issue in technical applications like oil field or fracking applications, this colour instability can prevent biosurfactants to win recognition in the technical field of for example cosmetics or household care.

It is thus an object of the invention to provide for colour stable biosurfactant compositions.

### DESCRIPTION OF THE INVENTION

It was found that, surprisingly, addition of low amounts of manganese can stabilize compositions comprising biosurfactants in terms of colour.

The present invention therefore provides compositions comprising biosurfactant and manganese at certain amounts.

The invention further provides a method to stabilize the colour of a composition comprising biosurfactant and the use of manganese to stabilize the colour of a composition comprising biosurfactant.

One advantage of the present invention is that the compositions according to the instant invention have a positive odour profile.

Another advantage of the present invention is that the compositions according to the instant invention have an improved colour stability. Colour change reactions could come from Maillard reaction, which is a chemical reaction between proteins or amino acids and sugars. Both reaction partners, proteins and sugars, are typically contained in small amounts in a fermentative produced biosurfactant like Rhamnolipids, Sophorolipids or Rubiwettin. These small concentrations are sufficient to have a colour change from light to dark.

A further advantage is that the compositions according to the instant invention have improved cleaning properties of surfaces.

Another advantage is that the compositions according to the instant invention have excellent foaming properties.

A further advantage of the compositions according to the instant invention is their reduced irritation for human skin.

Another advantage of the compositions according to the instant invention is their mildness and good physiological compatibility, in particular characterized by a high value in the red blood cell (RBC) test.

A further advantage of t the compositions according to the instant invention is their good skin feel during and after washing.

Another advantage of the compositions according to the instant invention is that they leave behind a smooth and soft skin feel after washing.

Another advantage is that the compositions according to the instant invention is their outstanding microbial stability.

Another advantage is that the compositions according to the instant invention have an improved ability to dissolve oils and fats.

Another advantage is that the composition according to the instant invention leads to a better chemical stability of the raw materials, which increases the shelf life.

Another advantage is that the composition according to the instant invention leads to a lower formation of side reactions and higher formulation purity.

Another advantage of the present invention is that the composition according to the instant invention shows especially good emulsification and dispersing properties.

One advantage of the present invention is that the compositions of the instant invention have a very good cleaning performance, especially in fatty stain removal from fabrics.

Another advantage of the present invention is that the compositions of the instant invention can be rinsed off from a surface or from fibres, that had been cleaned, very easily.

A further advantage is that the compositions of the instant invention support enzyme stability in terms of storage.

One advantage of the present invention is the homogeneity of the compositions of the instant invention even at low pH values.

Another advantage of the present invention is that the compositions of the instant invention are easier to dilute, especially in aqueous media.

Another advantage of the present invention is that the compositions of the instant invention are easily miscible with other surfactants.

A further advantage of the present invention is that the compositions of the instant invention can be incorporate into cosmetic formulations more easily.

Another advantage of the present invention is that the compositions of the instant invention allow the formulation of concentrated surfactant formulations, even at very low pH.

Another advantage of the present invention is that the compositions of the instant invention have a reduced foam tendency due to their high concentration and the low achievable pH value, thus simplifying transport and delivery.

Another advantage of the present invention is that the compositions of the instant invention allow for easy incorporation of hydrophobic components such as oils.

Another advantage of the present invention is that the compositions of the instant invention have a high storage stability.

A further advantage of the present invention is that the compositions of the instant invention cause less contamination during their production and transport in pipelines and, moreover, allow easier cleaning.

Another advantage of the present invention is that the compositions of the instant invention have a very low freezing point, which means that the compositions remain processable even at low temperatures.

Another advantage of the present invention is that the compositions of the instant invention, especially cosmetic or cleaning formulations, will remain colour stable.

Another advantage of the present invention is that the compositions of the instant invention, especially cosmetic or cleaning formulations, have a lower colour intensity.

The instant invention encompasses a composition comprising
A) 0.5 wt.-% to 90 wt. %, preferably 3.0 wt.-% to 85 wt. %, even more preferably 15 wt.-% to 80 wt. %, more preferably 25 wt.-% to 75 wt. %, most preferably 40 wt.-% to 70 wt. %, at least one biosurfactant, and
B) 0.01 ppm to 500 ppm, preferably 0.1 ppm to 200 ppm, more preferably 1.0 ppm to 50 ppm, most preferably 2.0 ppm to 19 ppm, manganese ions,
wherein the percentages by weight and the parts per million refer to the total composition.

Within the context of the present invention, “biosurfactants” are understood as meaning all glycolipids produced by fermentation. The term “biosurfactant” also covers glycolipids that are chemically or enzymatically modified after fermentation, as long as structurally a glycolipid remains. Raw materials for producing the biosurfactants that can be used are carbohydrates, in particular sugars such as e.g. glucose and/or lipophilic carbon sources such as fats, oils, partial glycerides, fatty acids, fatty alcohols, long-chain saturated or unsaturated hydrocarbons.

In the context of the present invention, the terms “surfactant” is understood to mean organic substances having interface-active properties that have the ability to reduce the surface tension of water at 20° C. and at a concentration of 0.5% by weight based on the overall composition to below 45 mN/m. Surface tension is determined by the DuNoüy ring method at 20° C.

Where average values are stated hereinbelow, then, unless stated otherwise, these are number-averaged average values.

Unless stated otherwise, percentages are data in percent by weight.

Unless stated otherwise, parts per million (ppm) are data in parts per million by weight.

Wherever measurement values are stated hereinbelow, then, unless stated otherwise, these have been determined at a temperature of 25° C. and a pressure of 1013 mbar.

The “pH” in connection with the present invention—unless stated otherwise—is defined as the value which is measured for the relevant composition at 25° C. after stirring for five minutes using a pH electrode calibrated in accordance with ISO 4316 (1977).

The biosurfactant comprised in the composition according to the instant invention preferably is an isolated biosurfactant.

As in the context of the instant invention the term “biosurfactant” has the meaning of a glycolipid produced by fermentation, the term “isolated biosurfactant” has the meaning as commonly used in the field of biotechnology.

The composition according to the instant invention preferably comprises as component A) at least one biosurfactant selected from rhamnolipids, sophorolipids, glucolipids, cellulose lipids, mannosylerythritol lipids and trehalose lipids, preferably rhamnolipids, sophorolipids and glucolipids, preferably rhamnolipids and glucolipids, most preferably rhamnolipids. The biosurfactants can be produced e.g. as in EP 0 499 434, U.S. Pat. No. 7,985,722, WO 03/006146, DE 19648439, DE 19600743, JP 01 304034, CN 1337439, JP 2006 274233, JP 2006 083238, JP 2006 070231, WO 03/002700, FR 2740779, DE 2939519, U.S. Pat. No. 7,556,654, FR 2855752, EP 1445302, JP 2008 062179 and JP 2007 181789 or the documents cited therein. Suitable biosurfactants can be acquired e.g. from Soliance, France.

Preferably, the composition according to the instant invention has, as biosurfactant at least one selected from rhamnolipids, in particular mono-, di- or polyrhamnolipids, glucolipids, in particular mono-, di- or polyglucolipids, and sophorolipids, in particular mono-, di- or polysophorolipids, preferably selected from rhamnolipids and glucolipids, most preferably rhamnolipids.

The term “rhamnolipids” in the context of the present invention preferably is understood to mean particularly compounds of the general formula (I) and salts thereof,

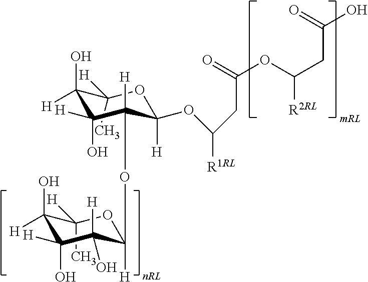

where
mRL=2, 1 or 0,
nRL=1 or 0,
R^{1RL} and R^{2RL}=mutually independently, identical or different, organic residues having 2 to 24, preferably 5 to 13 carbon atoms, in particular optionally branched, optionally substituted, particularly hydroxy-substituted, optionally unsaturated, in particular optionally mono-, bi- or tri-unsaturated alkyl residues, preferably those selected from the group consisting of pentenyl, heptenyl, nonenyl, undecenyl and tridecenyl and (CH₂)ₒ—CH₃ where o=1 to 23, preferably 4 to 12. If nRL=1, the glycosidic bond between the two rhamnose units is preferably in the α-configuration. The optically active carbon atoms of the fatty acids are preferably present as R-enantiomers (e.g. (R)-3-{(R)-3-[2-O-(α-L-rhamnopyranosyl)-α-L-rhamnopyranosyl]oxydecanoyl}oxydecanoate).

The term “di-rhamnolipid” in the context of the present invention is understood to mean compounds of the general formula (I) or salts thereof, where nRL=1.

The term “mono-rhamnolipid” in the context of the present invention is understood to mean compounds of the general formula (I) or salts thereof, where nRL=0.

Distinct rhamnolipids are abbreviated according to the following nomenclature:

“diRL-CXCY” are understood to mean di-rhamnolipids of the general formula (I) where nRL=1, in which one of the residues R^{1RL} and R^{2RL}=(CH₂)ₒ—CH₃ where o=X−4 and the remaining residue R^{1RL} or R^{2RL}=(CH₂)ₒ—CH₃ where o=Y−4.

“monoRL-CXCY” are understood to mean mono-rhamnolipids of the general formula (I), in which one of the residues R^{1RL} and R^{2RL}=(CH₂)ₒ—CH₃ where o=X−4 and the remaining residue R^{1RL} or R^{2RL}=(CH₂)ₒ—CH₃ where o=Y−4.

The nomenclature used therefore does not distinguish between “CXCY” and “CYCX”.

For rhamnolipids where mRL=0, monoRL-CX or diRL-CX is used accordingly.

If one of the abovementioned indices X and/or Y is provided with “:Z”, this signifies that the respective residue R^{1RL} and/or R^{2RL} is equal to an unbranched, unsubstituted hydrocarbon residue having X−3 or Y−3 carbon atoms having Z double bonds.

Methods for preparing the relevant rhamnolipids are disclosed, for example, in EP2786743 and EP2787065.

Rhamnolipids applicable in the context of the instant invention can also be produced by fermentation of Pseudomonas, especially Pseudomonas aeruginosa, which are preferably non genetically modified cells, a technology already disclosed in the eighties, as documented e.g. in EP0282942 and DE4127908. Rhamnolipids produced in Pseudomonas aeruginosa cells which have been improved for higher rhamnolipid titres by genetical modification can also be used in the context of the instant invention; such cells have for example been disclosed by Lei et al. in Biotechnol Lett. 2020 June; 42 (6): 997-1002.

Rhamnolipids produced by Pseudomonas aeruginosa are commercially available from Jeneil Biotech Inc., e.g. under the tradename Zonix, from Logos Technologies (technology acquired by Stepan), e.g. under the tradename NatSurFact, from Biotensidion GmbH, e.g. under the tradename Rhapynal, from AGAE technologies, e.g. under the name R90, R95, R95Md, R95Dd, from Locus Bio-Energy Solutions and from Shanghai Yusheng Industry Co. Ltd., e.g. under the tradename Bio-201 Glycolipids.

The present invention provides a composition preferably comprising as biosurfactant rhamnolipids, characterized in that the biosurfactant component A) comprises
51% by weight to 100% by weight, preferably 60% by weight to 95% by weight, particularly preferably 80% by weight to 90% by weight, of mono-rhamnolipids, especially those of formula (I) with nRL=0,
where the percentages by weight refer to the sum of all of the rhamnolipids present.

This surprisingly elevates the viscosity of the composition according to the instant invention. Preferably this preferred embodiment is combined with a content of soil release agents (see below), preferably of the carboxy methyl inulin type.

The present invention further provides a composition preferably comprising as biosurfactant rhamnolipids, characterized in that the biosurfactant component A) comprises
51% by weight to 95% by weight, preferably 55% by weight to 80% by weight, particularly preferably 60% by weight to 70% by weight, of diRL-C10C10,
where the percentages by weight refer to the sum of all of the rhamnolipids present.

A preferred composition according to the invention is characterized in that the composition comprises as biosurfactant rhamnolipids as described above with a content of
0.5% by weight to 15% by weight, preferably 3% by weight to 12% by weight, particularly preferably 5% by weight to 10% by weight, of diRL-C10C12: 1,
where the percentages by weight refer to the sum of all of the rhamnolipids present.

A further preferred composition according to the invention is characterized in that the composition comprises as biosurfactant rhamnolipids as described above with a content of
0.5 to 25% by weight, preferably 3% by weight to 15% by weight, particularly preferably 5% by weight to 12% by weight, of diRL-C10C12,
where the percentages by weight refer to the sum of all of the rhamnolipids present.

A further preferred composition according to the invention is characterized in that the composition comprises as biosurfactant rhamnolipids as described above with a content of
0.1% by weight to 25% by weight, preferably 2% by weight to 10% by weight, particularly preferably 4% by weight to 8% by weight, of diRL-C8C10,
where the percentages by weight refer to the sum total of all rhamnolipids present.

An even further preferred composition according to the invention is characterized in that the composition comprises as biosurfactant rhamnolipids as described above with a content of
0.1% by weight to 5% by weight, preferably 0.5% by weight to 3% by weight, particularly preferably 0.5% by weight to 2% by weight, of monoRL-C8C10 and/or, preferably and
0.1% by weight to 5% by weight, preferably 0.5% by weight to 3% by weight, particularly preferably 0.5% by weight to 2% by weight, of monoRL-C10C10,
where the percentages by weight refer to the sum of all of the rhamnolipids present.

The present invention provides a composition alternatively preferably comprising as biosurfactant rhamnolipids, characterized in that the biosurfactant component A) comprises
10% by weight to 50% by weight, preferably 20% by weight to 40% by weight, particularly preferably 25% by weight to 35% by weight, of monoRL-C10C10 and
where the percentages by weight refer to the sum of all of the rhamnolipids present.

The alternatively preferred composition according to the invention is preferably characterized in that the composition comprises as biosurfactant rhamnolipids as described above with a content of
10% by weight to 30% by weight, preferably 12% by weight to 25% by weight, particularly preferably 15% by weight to 20% by weight, of diRL-C10C10,
where the percentages by weight refer to the sum of all of the rhamnolipids present.

The alternatively preferred composition according to the invention is characterized in that the composition comprises as biosurfactant rhamnolipids as described above with a content of
10% by weight to 30% by weight, preferably 12% by weight to 25% by weight, particularly preferably 15% by weight to 20% by weight, of monoRL-C8C10,
where the percentages by weight refer to the sum of all of the rhamnolipids present.

The alternatively preferred composition according to the invention is characterized in that the composition comprises as biosurfactant rhamnolipids as described above with a content of
3% by weight to 25% by weight, preferably 5% by weight to 20% by weight, particularly preferably 10% by weight to 15% by weight, of monoRL-C10C12: 1,
where the percentages by weight refer to the sum total of all rhamnolipids present.

The alternatively preferred composition according to the invention is characterized in that the composition comprises as biosurfactant rhamnolipids as described above with a content of
1% by weight to 15% by weight, preferably 2% by weight to 10% by weight, particularly preferably 3% by weight to 8% by weight, of diRL-C10C12,
where the percentages by weight refer to the sum of all of the rhamnolipids present.

In the context of the present invention, the term “sophorolipids” preferably is understood as meaning compounds of the general formulae (IIa) and (IIb) and salts thereof

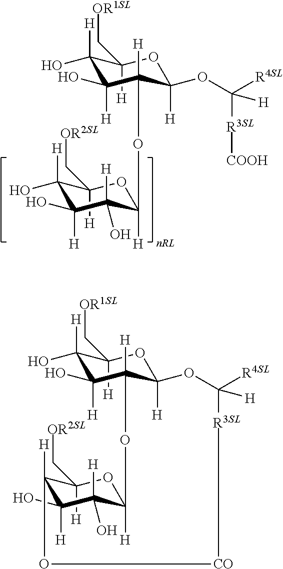

where
R^{1SL}=H or CO—CH₃,
R^{2SL}=H or CO—CH₃,
R^{3SL}=a divalent organic moiety which comprises 6 to 32 carbon atoms and which is unsubstituted or substituted by hydroxyl functions, is unbranched and optionally comprises one to three double or triple bonds,
R^{4SL}=H, CH₃ or a monovalent organic radical which comprises 2 to 10 carbon atoms and which is unsubstituted or substituted by hydroxyl functions, which is unbranched and which optionally comprises one to three double or triple bonds, and
nSL=1 or 0.

Sophorolipids may be used in accordance with the invention in their acid form or their lactone form. Preferred compositions according to the instant invention comprise a sophorolipid in which the ratio by weight of lactone form to acid form is in the range of 20:80 to 80:20, especially preferably in the ranges of 30:70 to 40:60.

To determine the content of sophorolipids in the acid or lactone form in a formulation, refer to EP1411111B1, page 8, paragraph [0053].

In connection with the present invention, the term “glucolipids” preferably is understood as meaning compounds of the general formula (III) and salts thereof,

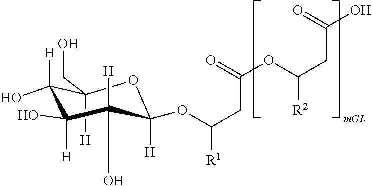

where
mGL=3, 2, 1 or 0, preferably 1 or 0,
R^{1GL} and R^{2GL}=independently of one another identical or different organic radical having 2 to 24 carbon atoms, in particular optionally branched, optionally substituted, in particular hydroxy-substituted, optionally unsaturated, in particular optionally mono-, di- or triunsaturated, alkyl radical, preferably one selected from the group consisting of pentenyl, heptenyl, nonenyl, undecenyl and tridecenyl and (CH₂)ₒ—CH₃ where o=1 to 23, preferably 4 to 12.

Distinct glucolipids are abbreviated according to the following nomenclature:

“GL-CXCY” is understood as meaning glucolipids of the general formula (III) in which one of the radicals R^{1GL} and R^{2GL}=(CH₂)ₒ—CH₃ where o=X−4 and the remaining radical R^{1GL} or R^{2GL}=(CH₂)ₒ—CH₃ where o=Y−4.

The nomenclature used thus does not differentiate between “CXCY” and “CYCX”.

If one of the aforementioned indices X and/or Y is provided with “:Z”, then this means that the respective radical R^{1GL} and/or R^{2GL}=an unbranched, unsubstituted hydrocarbon radical with X−3 or Y−3 carbon atoms having Z double bonds.

Methods for production of glucolipids can be carried out as described in WO2019154970.

When determining the content of component A) in the context of the present invention, the mass of the non-salt form is taken into account; thus, the weight of the corresponding cation is disregarded.

Within the context of the present invention, the content of component B) refers to manganese ions in solution and not to for example solid manganese salts in the composition.

To determine the content of manganese ions in the composition according to the instant invention preferably the methodology of inductively coupled plasma mass spectrometry (ICP-MS) is used.

The compositions according to the instant invention can bear high concentrations of the components A) and B) to be advantageously useful, e.g. in the production of end customer formulations.

In this context it is preferred in the instant invention, that the composition according to the instant invention is characterized in that it comprises
A) 25 wt.-% to 75 wt. %, more preferably 40 wt.-% to 70 wt. %, at least one biosurfactant, and
B) 0.1 ppm to 500 ppm, more preferably 0.5 ppm to 50 ppm, even more preferably 1.0 ppm to 19 ppm, manganese ions,
wherein the percentages by weight and the parts per million refer to the total composition.

The compositions according to the instant invention can bear concentrations of the components to be advantageously useful as end customer formulations.

In this context it is alternatively preferred in the instant invention, that the composition according to the instant invention is characterized in that it comprises
A) 0.5 wt.-% to 25 wt. %, more preferably 1.0 wt.-% to 20 wt. %, most preferably 3.0 wt.-% to 15 wt. %, at least one biosurfactant, and
B) 0.01 ppm to 500 ppm, more preferably 0.5 ppm to 50 ppm, even more preferably 1.0 ppm to 19 ppm, manganese ions,
wherein the percentages by weight and the parts per million refer to the total composition.

This alternatively preferred composition according to the instant invention is preferably a pharmaceutical, especially dermatological, or cosmetic formulation.

These alternatively preferred composition according to the instant invention is alternatively preferably a household care formulation, for example a cleaning formulation or laundry formulation.

The compositions according to the instant invention preferably comprise water, preferably in an amount of 5 wt.-% to 95 wt.-%, more preferably of 20 wt.-% to 80 wt.-%, more preferably of 30 wt.-% to 70 wt.-%, wherein the weight percentages refer to the total composition.

The composition according to the instant invention preferably has a pH of 2.0 to 12.5, preferably of 5.0 to 10.0, particularly preferably of 5.5 to 9.0.

If the composition according to the instant invention is used, for example, in laundry detergents, it preferably has a pH of 7.0 to 12.5, preferably of 7.5 to 12.0, particularly preferably of 8.0 to 12.0.

If the composition according to the instant invention is used, for example, in manual dishwashing or cosmetic agents, it preferably alternatively has a pH of 4 to 8, preferably of 4.5 to 7.5, particularly preferably of 5 to 6.5.

The composition according to the instant invention preferably has a Gardner colour value in the range of from 0.01 to 15, preferably of from 0.5 to 8, more preferably 1 to 5.

The compositions according to the instant invention preferably comprise
C) at least one preservative.

The compositions according to the instant invention preferably is characterized in that said preservative is selected from the group comprising, preferably consisting of,
p-anisic acid, benzoic acid, levulinic acid, sorbic acid, lactic acid, mandelic acid, salicylic acid, dehydroacetic acid, caprylhydroxamic acid, cinnamic acid, geranic acid, pelargonic acid, and the salts of the aforementioned acids,
methylparaben, ethylparaben,
phenylpropanol, benzyl alcohol, phenethyl alcohol, phenoxyethanol,
propanediol, pentylene glycol, 1,2-hexanediol, caprylyl glycol, undecyl alcohol, methylpropanediol, ethylhexylglycerin, n-octylglycerin, n-heptylglycerin, n-hexylglycerin, methylheptylglycerin, undecylenamidopropyltrimonium methosulfate,
isothiazolinones, preferably selected from chloromethylisothiazolinone (CIT), methylisothiazolinone (MIT), benzisothiazolinone (BIT), butylbenzisothiazolinone (BBIT),
triethyl citrate,
citral,
hydroxymethylglycinate, glyceryl caprylate,
iodopropynyl butylcarbamat (IPBC), and
Sodium Caproyl/Lauroyl Lactylate.

The composition according to the instant invention can be formulated as a cosmetic composition; preferably in this instance the following applies:

The compositions according to the invention preferably comprise at least one additional component selected from the group of
cosmetic active ingredients,
emollients,
emulsifiers,
thickeners/viscosity regulators/stabilizers,
UV light protection filters,
antioxidants,
hydrotropes,
solids and fillers,
film formers,
pearlescence additives,
deodorant and antiperspirant active ingredients,
insect repellents,
self-tanning agents,
conditioning agents,
perfumes,
dyes,
odour absorbers,
superfatting agents,
other solvents,
preferably cosmetic active ingredients.

Substances which can be used as exemplary representatives of the individual groups are known to those skilled in the art and can be found for example in German application DE 102008001788.4. This patent application is hereby incorporated as reference and thus forms part of the disclosure.

As regards further optional components and the amounts used of these components, reference is made expressly to the relevant handbooks known to those skilled in the art, for example K. Schrader, “Grundlagen und Rezepturen der Kosmetika [Cosmetics—fundamentals and formulations]”, 2nd edition, pages 329 to 341, Hüthig Buch Verlag Heidelberg.

The amounts of the particular additives are determined by the intended use.

Typical boundary formulations for the respective applications are known prior art and are contained for example in the brochures of the manufacturers of the particular base and active ingredients. These existing formulations can generally be adopted unchanged. However, if required, for adjustment and optimization, the desired modifications can be undertaken by simple tests without complication.

The composition according to the instant invention can be formulated as a household care composition; preferably in this instance the following applies:

The composition according to the instant invention preferably comprises one or more auxiliary agents selected from the group consisting of bleaching systems, hydrotropes, polymers, which may be synthetic, biopolymers, anti-redeposition aids, fiber protection agents, soil release agents, dye transfer inhibitors, fabric hueing agents, opacifiers, blueing dyes, enzyme stabilizing agents, solvents, viscosity modifiers, preservatives, pH-regulators and salts like NaCl and Na₂SO₄.

A further aspect of the instant invention is a method of stabilizing the colour of a composition comprising a biosurfactant comprising the steps
a) providing a composition comprising a biosurfactant, and
b) adding manganese to the composition to a concentration of 0.01 ppm to 500 ppm,
preferably 0.10 ppm to 200 ppm, more preferably 1.0 ppm to 50 ppm, most preferably 2.0 ppm to 19 ppm, manganese ions, wherein the parts per million refer to the total composition.

A preferred method according to the instant invention is characterized in that the manganese is added in the form of manganese oxide, manganese hydroxide and/or a manganese salt, preferably as aqueous solution, wherein the oxide is preferably selected from the group of MnO₂, Mn₃O₄, Mn₂O₃, MnO, Mn₂O₇ and MnO₃ and the salt is preferably selected from the group of MnSO₄, MnCl₂, Mn(CH₃CO₂)₂, MnCO₃, Mn(NO₃)₂, manganese gluconate and manganese permanganates, e.g. KMnO₄.

Preferably the biosurfactant comprised in the provided composition of step a) of the method of the instant invention is an isolated biosurfactant.

A preferred method according to the instant invention is characterized in that said biosurfactant is selected from the group of rhamnolipids, glucolipids and sophorolipids, preferably rhamnolipids and glucolipids, most preferably rhamnolipids.

A preferred method according to the instant invention is characterized in that said composition comprising a biosurfactant provided in step a) has a Gardner colour value in the range of from 0.01 to 15, preferably of from 0.5 to 8, more preferably 1 to 5.

A suited methodology for determination of the Gardner colour value is the third edition of ISO 4630-2015 Clear liquids—Estimation of colour by the Gardner colour scale.

In the case of turbid material, such as for example an emulsion, a different methodology for colour determination needs to be utilized.

The CIELAB colour space is defined by the L*a*b values wherein the brightness L is the relevant indicator for colour.

A preferred method according to the instant invention is characterized in that said composition comprising a biosurfactant provided in step a) has an L colour value of the CIELAB colour space in the range of from 20 to 100, preferably of from 30 to 95, more preferably 50 to 90.

A suited methodology for determination of the L colour value of the CIELAB colour space is the measurement of surface reflexion using the spectrophotometer Konica-Minolta CM-700d.

In the method according to the instant invention, particularly preferred embodiments of the compositions according to the instant invention and preferred components thereof are equally preferably used in the context of the method according to the instant invention, also regarding their amounts.

A further aspect of the instant invention is the use of manganese ions to improve the colour stability of a composition comprising at least one biosurfactant.

A further aspect of the instant invention is the use of manganese ions to prevent malodour of a composition comprising at least one biosurfactant.

In the case of the uses according to the instant invention, particularly preferred embodiments of the compositions and method according to the instant invention and preferred components thereof are equally preferably used in the context of the uses according to the instant invention, also regarding their amounts.

The examples adduced hereinafter describe the present invention by way of example, without any intention that the invention, the scope of application of which is apparent from the entirety of the description and the claims, be restricted to the embodiments specified in the examples.

### BRIEF DESCRIPTION OF THE FIGURES

FIG. 1: Dose dependent colour stabilization of 50% rhamnolipids by MnSO₄

FIG. 2: Dose dependent colour stabilization of 15% rhamnolipids by MnSO₄

FIG. 3: Dose dependent colour stabilization of 15% rhamnolipids by manganese oxides

FIG. 4: Dose dependent colour de-stabilization of 15% rhamnolipids by iron and copper ions

FIG. 5: Dose dependent colour stabilization of 50% glucolipids by MnSO₄

### EXAMPLES

### Example 1: Dose Dependent Colour Stabilization of a 50% Rhamnolipid Solution by MnSO₄

Rhamnolipids were prepared as described in example 1 of EP3023431.

MnSO₄×H₂O was added to a 50 wt. % rhamnolipid solution in water from a 1% (w/w) pre-dilution in water to the final concentration of 1 ppm and 100 ppm Mn²⁺ and mixed well.

The sample was filled into an air-tight container and incubated at 45° C.

At the indicated timepoints, samples were taken and measured spectrophotometrically according to ISO 4630.

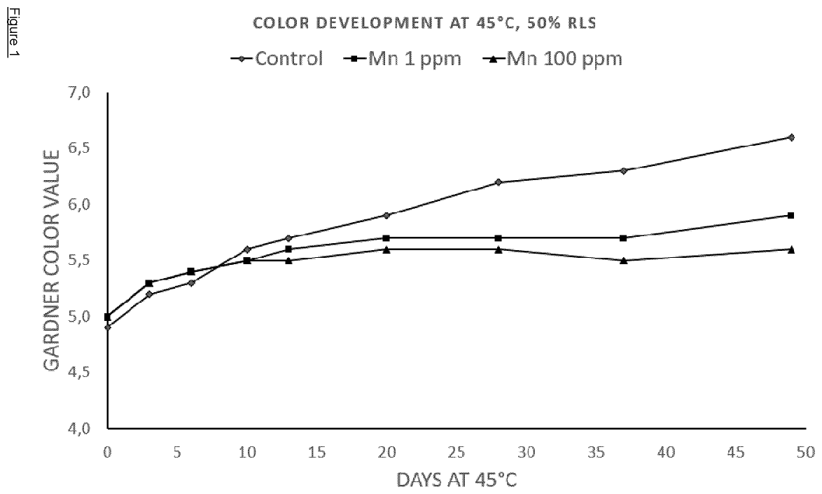

The results (Gardener) are shown in FIG. 1.

### Example 2: Dose Dependent Colour Stabilization of a 15% Rhamnolipid Solution by MnSO₄

Rhamnolipids were prepared as described in example 1 of EP3023431.

MnSO₄×H₂O was added to a 15 wt. % rhamnolipid solution in water from a 1% (w/w) pre-dilution in water to the final concentration of 1 ppm and 100 ppm Mn²⁺ and mixed well.

The sample was filled into an air-tight container and incubated at 45° C.

At the indicated timepoints, samples were taken and measured spectrophotometrically according to ISO 4630.

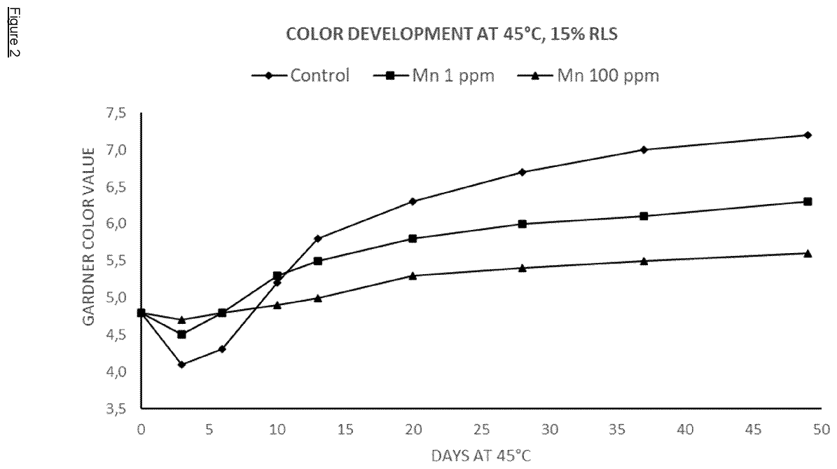

The results (Gardner) are shown in FIG. 2.

### Example 3: Colour Stabilization of a 15% Rhamnolipid Solution by Different Manganese Oxides

Rhamnolipids were prepared as described in example 1 of EP3023431.

Manganese oxides were added to 15 wt. % rhamnolipid solution in water in concentrations of 500 and 3000 ppm and incubated under stirring for 60 min at room temperature. Subsequently, manganese oxides were separated by filtration using a pressure strainer (1 bar) with K800 filter discs (Seitz PALL).

As a result, compositions with low concentrations (<10 ppm) of Mn²⁺ are obtained.

The sample was filled into an air-tight container and incubated at 45° C.

At the indicated timepoints, samples were taken and measured spectrophotometrically according to ISO 4630.

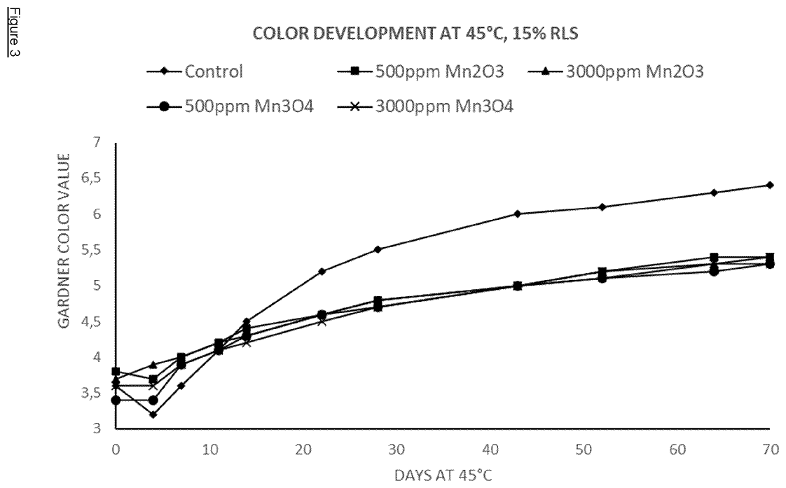

The results (Gardner) are shown in the following table and FIG. 3.

| | | added | | | | | | | | | | | |
|---|---|---|---|---|---|---|---|---|---|---|---|---|---|
| | manganese | amount of | | | | | | | | | | | |
| | oxide | manganese | days | | | | | | | | | | |
| Variant | added | oxide (ppm) | 0 | 4 | 7 | 11 | 14 | 22 | 28 | 43 | 52 | 64 | 70 |
| Control | none | | 3.6 | 3.2 | 3.6 | 4.1 | 4.5 | 5.2 | 5.5 | 6 | 6.1 | 6.3 | 6.4 |
| 500 ppm | Mn₂O₃ | 500 | 3.8 | 3.7 | 4 | 4.2 | 4.4 | 4.6 | 4.8 | 5 | 5.2 | 5.4 | 5.4 |
| Mn₂O₃ | | | | | | | | | | | | | |
| 3000 ppm | Mn₂O₃ | 3000 | 3.7 | 3.9 | 4 | 4.2 | 4.3 | 4.6 | 4.8 | 5 | 5.1 | 5.3 | 5.3 |
| Mn₂O₃ | | | | | | | | | | | | | |
| 500 ppm | Mn₃O₄ | 500 | 3.4 | 3.4 | 3.9 | 4.1 | 4.3 | 4.6 | 4.7 | 5 | 5.1 | 5.2 | 5.3 |
| Mn₃O₄ | | | | | | | | | | | | | |
| 3000 ppm | Mn₃O₄ | 3000 | 3.6 | 3.6 | 3.9 | 4.1 | 4.2 | 4.5 | 4.7 | 5 | 5.2 | 5.3 | 5.4 |
| Mn₃O₄ | | | | | | | | | | | | | |

### Example 4 (not According to the Invention): Dose Dependent Colour Impact of Iron and Copper Salts on a 15% Rhamnolipid Solution

Rhamnolipids were prepared as described in example 1 of EP3023431.

FeSO₄*7H₂O or CuSO₄*5H₂O was added to the di-Rhamnolipid material from a 1% (w/w) pre-dilution to the final concentration and mixed well. The sample was filled into an air-tight container and incubated at 45° C. At the indicated timepoints, samples were taken and measured spectrophotometrically according to ISO 4630.

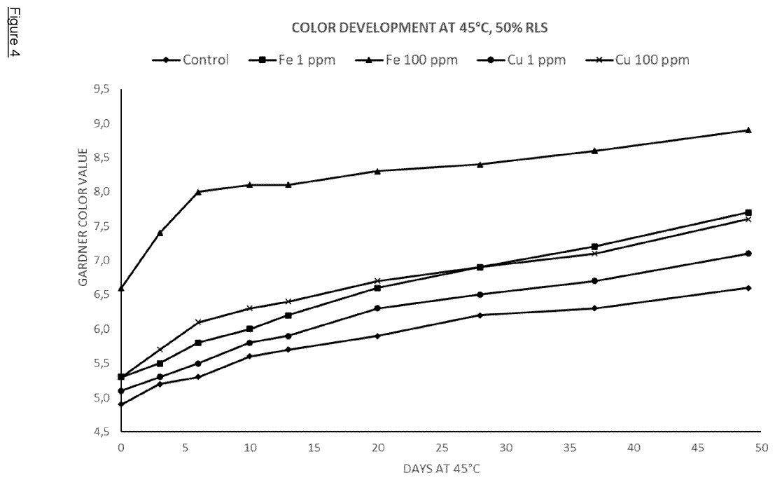

The results (Gardner) are shown in FIG. 4.

### Example 5: Dose Dependent Colour Stabilization of a 50% Glucolipid Solution by MnSO₄

Glucolipids were prepared as described WO2019/154970.

MnSO₄×H₂O was added to a 50 wt. % glucolipid solution in water from a 1% (w/w) pre-dilution in water to the final concentration of 10 ppm and 25 ppm Mn²⁺ and mixed well.

The sample was filled into an air-tight container and incubated at 45° C.

At the indicated timepoints, samples were taken and measured spectrophotometrically according to ISO 4630.

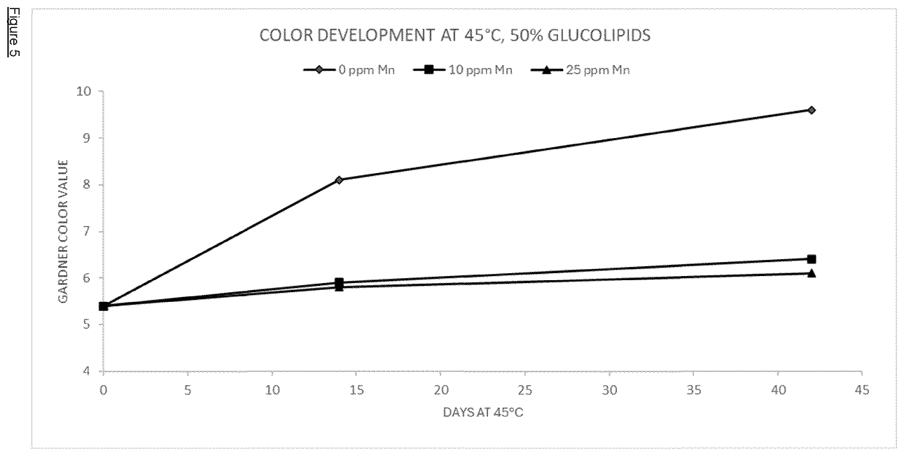

The results (Gardener) are shown in FIG. 5.

### Example 6: The Use of MnSO₄ to Prevent Malodour of a Biosurfactant Composition

Rhamnolipids were prepared as described in example 1 of EP3023431.

MnSO₄×H₂O was added to a 50 wt. % rhamnolipid solution in water from a 1% (w/w) pre-dilution in water to the final concentration of 0 ppm and 10 ppm Mn²⁺ and mixed well.

The sample was then filled into an air-tight container and incubated at 45° C. for 50 days.

The impact of manganese on sensory properties of the rhamnolipid composition was tested by odour evaluation using a panel of trained panelists.

By “trained panelists” we mean here individuals that had previously been screened for olfactive acuity and were experienced comparing biosurfactant samples. Moreover, the panelists were prior acquainted with the malodor sample before carrying out the evaluation of the samples.

The results are shown in the following table on a scale from 1 to 5 concerning the odor property “rancid”.

| | t = 0 days | t = 50 days |
|---|---|---|
| 0 ppm Mn²⁺ | 1.5 | 3.2 |
| 10 ppm Mn²⁺ | 1.6 | 2.2 |

### Example 7: Formulation Examples

Rhamnolipids (RLs) were prepared as described in example 1 of EP3023431.

Mono-Rhamnolipids (mRLs) were prepared as described in example 2 of EP3672690.

The sophorolipid (SLs) used is a sophorolipid REWOFERM® SL ONE from Evonik, which has a lactone to acid ratio of 40:60.

Glucolipids (GLs) were produced according to example 2 of WO2019154970 via fermentation.

As manganese source MnSO₄×H₂O was used.

The following compositions were prepared in water:

| | SF1 | SF2 | SF3 | SF4 | SF5 | SF6 | SF7 | SF8 | SF9 | SF10 |
|---|---|---|---|---|---|---|---|---|---|---|
| RLs/wt.-% | 40 | 40 | — | — | — | — | — | — | 16 | 16 |
| mRLs/wt.-% | — | — | 35 | 35 | — | — | — | — | 4 | 4 |
| SLs/wt.-% | — | — | — | — | 40 | 40 | — | — | 10 | 10 |
| GLs/wt.-% | — | — | — | — | — | — | 40 | 40 | 10 | 10 |
| Mn²⁺/ppm | 5 | 400 | 5 | 400 | 5 | 400 | 5 | 400 | 5 | 400 |

Each of the stock solution SF1 to SF10 is used to prepare the formulations listed below, where SFX stands for the respective stock solution; thus, 10 different formulations are explicitly disclosed in each recipe below.

Recipes 1a: Shower Cream

| Ingredients [wt. %] | 1a |
|---|---|
| Water | ad 100.0 |
| Sodium Laureth Sulfate (Texapon NSO, BASF, 28%) | 25.0 |
| Coco-Glucoside (Plantacare 818 UP, BASF, 51%) | 8.0 |
| Cocamidopropyl Betaine (TEGO ® Betain F 50, Evonik, | 8.0 |
| 38%) | |
| SFX | 2.3 |
| PEG-18 Glyceryl Oleate/Cocoate (ANTIL ® 171, Evonik) | 1.5 |
| Sorbitan Sesquicaprylate (ANTIL ® Soft SC, Evonik) | 0.8 |
| Glyceryl Oleate (TEGIN ® O V, Evonik) | 0.8 |
| Perfume Spicy Herbs (IFF) | 0.2 |
| Polyglyceryl-4 Caprate (TEGOSOFT ® PC 41, Evonik) | 0.6 |
| Helianthus Annuus Seed Oil (AEC Sunflower Oil, A & E | 0.2 |
| Connock, Perfumery & Cosmetics Ltd.) | |
| Linalool (Lipofresh, Lipo Chemicals, Inc.) | 0.1 |
| Coumarin (Rhodiascent extra pure, Solvay Rhodia) | 0.1 |
| Glycerin (Glycerol EP, vegetable, Spiga Nord) | 0.4 |
| Hydroxypropyl Methylcellulose (TEGOCEL ® HPM 50, | 0.2 |
| Evonik) | |
| Glycol Distearate (TEGIN ® G 1100 Pellets, Evonik) | 0.4 |
| Sodium Chloride | 0.5 |
| Hydroxypropyl Guar Hydroxypropyltrimonium Chloride | 0.2 |
| (Jaguar C-162, Solvay Rhodia) | |
| dermofeel TOCO 70 non GMO | 0.1 |
| Preservative | q.s. |
| Citric Acid | ad |
| | pH 5.2 |

Recipes 2a, 2b: Body Shampoo

| | Ingredients [wt. %] | 2a | 2b |
|---|---|---|---|
| Phase A | SFX | 8 | 4 |
| | Lavandula Angustifolia (Lavender) Oil | 0.2 | 0.2 |
| | (AEC Lavender Oil, A&E Connock | | |
| | Ltd.) | | |
| | Perfume | 0.1 | 0.1 |
| Phase B | Sodium Cocoamphoacetate | 10.0 | 10.0 |
| | (REWOTERIC ® AM C, Evonik, 32%) | | |
| Phase C | Water | ad 100.0 | ad 100.0 |
| | Xanthan Gum (Keltrol CG-SFT, CP | 1.2 | 1.2 |
| | Kelco) | | |
| Phase D | Sodium Lauroyl Methyl Isethionate | 4.5 | 4.5 |
| | (Iselux, Innospec Active Chemicals) | | |
| | Capryl/Capramidopropyl Betaine | 4.5 | 4.5 |
| | (TEGO ® Betaine 810, Evonik, 38%) | | |
| | Citric Acid | 1.2 | 1.2 |
| Phase E | Water | 10.0 | 10.0 |
| | Polyquaternium-7 (Merquat 550, Nalco) | 0.4 | 0.4 |
| | Preservative | q.s. | q.s. |

Recipes 3a: Shampoo

| | Ingredients [wt. %] | 3a |
|---|---|---|
| Phase A | SFX | 2.0 |
| | Isopropyl Myristate (TEGOSOFT ® M, Evonik) | 0.2 |
| | Perfume | 0.1 |
| Phase B | Water | ad 100.0 |
| Phase C | Sodium Lauryl Sulfate (Texapon LS 35, BASF, | 28.0 |
| | 30%) | |
| Phase D | Cocamidopropyl Betaine (TEGO ® Betain F 50, | 9.0 |
| | Evonik, 38%) | |
| Phase E | Cocamide MEA (REWOMID ® C 212, Evonik) | 2.0 |
| | Xanthan Gum (Keltrol CG-SFT, CP Kelco) | 0.3 |
| | Water | 10.0 |
| Phase F | Water | 10.0 |
| | Polyquaternium-10 (Polymer JR 400, Amerchol) | 0.2 |
| Phase G | Citric Acid | ad pH 5.0 |
| | Preservative | q.s. |

Recipes 4a: Shampoo

| Ingredients [wt. %] | 4a |
|---|---|
| Water | ad 100.0 |
| Cocamidopropyl Betaine (TEGO ® Betain F 50, Evonik, | 22.0 |
| 38%) | |
| SFX | 8.0 |
| Sodium Cocoyl Glutamate (Plantapon ACG HC, BASF) | 1.5 |
| Sodium Cocoyl Glycinate (Hostapon SG, Clariant) | 0.8 |
| Zinc Pyrithione (Microcare ZP, Thor) | 0.1 |
| PEG-120 Methyl Glucose Dioleate (ANTIL ® 120 Plus, | 0.4 |
| Evonik) | |
| Sodium Chloride | 0.5 |
| Isostearamide MIPA; Glyceryl Laurate (ANTIL ® SPA 80, | 0.5 |
| Evonik) | |
| Xanthan Gum (Keltrol CG-SFT, CP Kelco) | 0.3 |
| Hydroxypropyl Guar Hydroxypropyltrimonium Chloride | 0.3 |
| (Jaguar C-162, Solvay Rhodia) | |
| Quaternium-80 (ABIL ® Quat 3272, Evonik) | 0.4 |
| Palmitamidopropyltrimonium Chloride (VARISOFT ® | 0.4 |
| PATC, Evonik) | |
| Argania Spinosa Oil (Argan Oil, DSM Nutritional Products | 0.1 |
| Ltd.) | |
| Glycerin (Glycerol EP, vegetable, Spiga Nord) | 0.6 |
| Tetrasodium EDTA (Versene 100, The Dow Chemical | 0.1 |
| Company) | |
| Caffeine (Merck KGaA/EMD Chemicals, Inc.) | 0.1 |
| Hydrolyzed Wheat Protein (Gluadin WLM, BASF) | 0.1 |
| Limonene (Dipentene No. 122, Hercules Inc.) | 0.1 |
| Citric Acid | ad pH 5.5 |
| Sodium Phytate; Aqua; Alcohol (dermofeel@ PA-3; Evonik | |
| Dr. Straetmans GmbH (PA-3) | |
| Perfume | 0.2 |
| Aqua; Sodium Levulinate; Sodium Benzoate (Verstatil ® BL | |
| non GMO; Evonik Dr. Straetmans GmbH) | |

Recipes 5a: Liquid Soap

| Ingredients [wt. %] | 5a |
|---|---|
| Water | ad 100 |
| Glycerin (Glycerol EP, vegetable, Spiga Nord) | 4.0 |
| Alcohol | 4.0 |
| Sodium Coco-Sulfate (Texapon HC G, BASF) | 3.0 |
| SFX | 4 |
| Xanthan Gum (Keltrol CG-SFT, CP Kelco) | 1.5 |
| Mangifera Indica (Mango) Fruit Extract (Mango | 0.5 |
| Extract, Draco Natural Products) | |
| Limonene (Dipentene No. 122, Hercules Inc.) | 0.1 |
| Linalool (Lipofresh, Lipo Chemicals, Inc.) | 0.1 |
| Citric Acid | ad pH 4.9 |
| Preservative | q.s. |
| Dyes | q.s. |

Recipes 6a: Liquid Soap

| | Ingredients [wt. %] | 6a |
|---|---|---|
| Phase A | Zinc Ricinoleate; Tetrahydroxyproyl | 0.80 |
| | Ethylenediamine; Laureth-3; Propylene | |
| | Glycol (TEGODEO ® CW 90 E MB, Evonik) | |
| | Polyglyceryl-3 Caprylate (TEGO ® Cosmo P 813 | 0.50 |
| | MB, Evonik) | |
| | Aqua; Sodium Laureth Sulfate (Emal 228HP, Kao) | 26.00 |
| | Perfume | 0.30 |
| | Water, deionized | ad 100 |
| | Cocamidopropyl Betaine (TEGO ® Betain F 50, | 8.50 |
| | Evonik) | |
| | Lauryl Glucoside, 50% (Plantacare 1200 UP, BASF) | 3.25 |
| | Aqua, Sodium Levulinate; Potassium Sorbate | 2.00 |
| | (Verstatil ® SL; Evonik) | |
| | SFXs | 0.80 |
| Phase B | Sodium Chloride | |

Recipes 7a: Cream Soap

| Ingredients [wt. %] | 7a |
|---|---|
| Water | ad 100 |
| Propylene Glycol (Euxyl K 320, Schülke & Mayr GmbH) | 2.0 |
| Coco-Glucoside (Plantacare 818 UP, BASF, 51) | 10.0 |
| Glycerin (Glycerol EP, vegetable, Spiga Nord) | 5.0 |
| SFX | 1.25 |
| Disodium Cocoyl Glutamate (Planatpon ACG LC, BASF) | 2.5 |
| Xanthan Gum (Keltrol CG-SFT, CP Kelco) | 1.2 |
| Stearic Acid (Pristerene 4922, Croda Europe, Ltd.) | 1.2 |
| Citric Acid | ad pH 5.5 |
| Olea Europaea Fruit Oil (Cropure Olive, Croda Europe, Ltd.) | 0.2 |
| Glyceryl Oleate (TEGIN ® O V, Evonik) | 1.0 |
| Sodium Cocoyl Glutamate (Plantapon ACG HC, BASF) | 0.8 |
| Perfume | 0.1 |
| Preservative | q.s. |

Recipes 8a: Oil Bath

| Ingredients [wt. %] | 8a |
|---|---|
| Water | ad 100.0 |
| Glycine Soja Oil (Cropure Soybean, Croda Europe, Ltd.) | 20.0 |
| SFX | 6.0 |
| Polyglyceryl-3 Palmitate (Dermofeel ® PP, Evonik Dr. | 4.5 |
| Straetmans) | |
| Glyceryl Caprylate (Dermosoft ® GMCY, Evonik Dr. | 3.0 |
| Straetmans) | |
| Simmondsia Chinensis Seed Oil (AEC Jojoba Oil Refined, | 1.2 |
| A & E Connock, Perfumery & Cosmetics Ltd.) | |
| Prunus Amygdalus Dulcis (Sweet Almond) Oil (Cropure | 1.0 |
| Almond, Croda Europe, Ltd.) | |
| Triticum Vulgare Germ Oil (Cropure Wheatgerm, Croda | 0.5 |
| Europe, Ltd.) | |
| Tocopherol (Euxyl K 700, Schülke & Mayr GmbH) | 0.2 |
| Limonene (Dipentene No. 122, Hercules Inc.) | 0.1 |
| Citral | 0.1 |
| Preservative | q.s. |
| Dyes | q.s. |

Recipe 9a, 9b: Oil Bath

| | Ingredients [wt. %] | 9 |
|---|---|---|
| Phase A | C12-C15 Alkyl Benzoate (TEGOSOFT ® TN, | 29.5 |
| | Evonik) | |
| | Helianthus Annuus (Sunflower) Seed Oil | 26.0 |
| | Paraffinum Perliquidum | 30.0 |
| | Diethylhexyl Sodium Sulfosuccinate (TEGO ® | 9.0 |
| | Sulfosuccinate DO 75; Evonik | |
| | SFX | 2 |
| | Polyglyceryl-3 Caprate (TEGOSOFT ® PC 31 MB; | 5.0 |
| | Evonik) | |
| Phase B | Water | ad 100 |
| Phase Z | Preservative | q.s. |

Recipes 10a: Micellar Water for make-up removal

| Ingredients [wt. %] | 10a |
|---|---|
| Water | ad 100.0 |
| Perfume | 0.1 |
| SFX | 2.5 |
| Polyglyceryl-6 Caprylate; Polyglyceryl-3 Cocoate; | 1.0 |
| Polyglyceryl-4 Caprate; Polyglyceryl-6 Ricinoleate | |
| (TEGO ® Solve 61, Evonik) | |
| Betaine (TEGO ® Natural Betaine, Evonik) | 2.0 |
| Glycerin (Glycerol EP, vegetable, Spiga Nord) | 1.0 |
| Preservative | q.s. |

Recipes 11a: Natural Micellar Water

| | Ingredients [wt. %] | 11a |
|---|---|---|
| Phase A | Caprylyl/Capryl Glucoside; Aqua, Sodium Cocoyl | 2.5 |
| | Glutamate; Glyceryl Caprylate; Citric Acid; | |
| | Polyglyceryl-6 Oleate; Sodium Surfactin | |
| | (symbio ®solv clear plus MB, Evonik) | |
| | Perfume | 0.3 |
| | SFXs | 2 |
| Phase B | Glycerin | 5.0 |
| | Betaine (TEGO ® Natural Betaine; Evonik) | 2.0 |
| | Phenylpropanol (dermosoft ® 250 eco; Evonik) | 0.4 |
| | Pentylene Glycol (dermosoft ® Pentiol eco; Evonik) | 2.0 |
| | Sodium Phytate; Aqua; Alcohol (dermofeel ® PA-3; | 0.1 |
| | Evonik) | |
| | Water, deionized | ad 100 |

Recipes 12a: Micellar Water

| Ingredients [wt. %] | 12a |
|---|---|
| SFXs | 5 |
| Water | ad 100 |
| Betaine (TEGO ® Natural Betaine; Evonik) | 2.0 |
| Pentylene Glycol (dermosoft ® Pentiol eco; Evonik) | 4.0 |
| Sodium Anisate (dermosoft ® anisate eco; Evonik) | 0.3 |
| Glycerin | 1.0 |
| Perfume | q.s. |

Recipes 13a: Skin Tonic

| | Ingredients [wt. %] | 13a |
|---|---|---|
| Phase A | Water, deionized | ad 100 |
| | Sodium PCA; Aqua (Ajidew NL-50N; Ajinomoto) | 0.5 |
| | Glycerin | 4.0 |
| Phase B | SFXs | 2 |
| | Sodium Caproyl/Lauroyl Lactylate; Triethyl Citrate | 1.0 |
| | (dermosoft ® decalact liquid; Evonik) | |
| | Alcohol denat. | 25.0 |
| | Perfume | 0.3 |
| | Triethyl Citrate (dermofeel ® TEC eco; Evonik) | 2.0 |

Recipes 14a: Scalp Serum

| | Ingredients [wt. %] | 14a |
|---|---|---|
| Phase A | Water | ad 100 |
| | Propylene Glycol | 1.0 |
| | Xanthan Gum (Keltrol CG-SFT) | 0.2 |
| Phase B | SFXs | 0.6 |
| Phase C | Ceramide NG; Olea Europaea (Olive) Fruit Oil; | 1.0 |
| | Ricinus Communis (Castor) Seed Oil; Evonik) | |
| | Phenoxyethanol; Caprylyl Glycol (Verstatil ® PC; | 1.0 |
| | Evonik) | |
| | Polyglyceryl-3 Caprylate (TEGO ® Cosmo P 813 | 1.0 |
| | MB; Evonik) | |
| Phase D | Citric Acid (10% sol.) | q.s. |

Recipes 15a: Micellar Cleansing Foam

| | Ingredients [wt. %] | 15a |
|---|---|---|
| Phase | Polyglyceryl-Caprylate; Polyglyceryl-3 Cocoate; Polyglyceryl- | 4.0 |
| A | 4 Caprtar; Polyglyceryl-6 Ricinoleate (TEGO ® Solve 61 MB, | |
| | Evonik | |
| | SFX | 1 |
| | Prunus Amygdalus Dulcis (Sweet Almond) Oil | 0.2 |
| | Caprylyl/Capryl Glucoside; Aqua; Sodium Cocoyl Glutamate; | 1.67 |
| | Glyceryl Caprylate; Citric Acid; Polyglyceryl-6 Oleate; | |
| | Sodium Surfactin (symbio ®solv clear plus MB, Evonik) | |
| Phase | Bisabolol | 0.1 |
| B | Water | ad 100 |
| | Glycerin | 3.0 |
| | Benzyl Alcohol; Caprylyl Glycol; Benzoic Acid (Verstatil ® | 1.0 |
| | BOB, Evonik) | |
| | NaOH (10%) | Ad |
| | | pH 5.5 |

Recipes 16a: Liquid Facial Cleanser

| Ingredients [wt. %] | 16a |
|---|---|
| SFXs | 3 |
| Polyglyceryl-4 Caprate (TEGOSOFT ® PC 41 MB; Evonik) | 2.0 |
| Benzyl Alcohol; Caprylyl Glycol; Benzoic Acid (Verstatil ® BOB, | 2.0 |
| Evonik) | |
| Water | ad 100 |
| Glycerin | 2.0 |
| 1,2-Hexanediol (dermosoft ® Hexiol) | 1.4 |
| Sodium Hydroxide (25% sol) | q.s. |

Recipes 17a: Biphasic Make-up Remover

| | Ingredients [wt. %] | 17a |
|---|---|---|
| Phase | Water, deionized | ad 100 |
| A | Glycerin (Glycerin 99.5%, Ph. Eur .; Cremer Oleo) | 10.0 |
| | Glycerin; Aqua; Sodium Levulinate; Sodium Anisate | 3.5 |
| | (dermosoft ® 1388 eco) | |
| | Polyglyceryl-10 Laurate; Aqua; Citric Acid | 0.45 |
| | (dermofeel ® G 10 L 70 MB; Evonik) | |
| | SFXs | 1 |
| | Sodium Chloride | 1.0 |
| Phase | Glyceryl Caprylate (dermosoft ® GMCY MB; Evonik) | 0.25 |
| B | Helianthus annuus seed oil (Sunflower oil) (Henry | 42.0 |
| | Lamotte Oils GmbH) | |
| | Tocopherol; Helianthus Annuus (Sunflower) Seed | 0.1 |
| | Oil (derrmofeel Toco 70 non GMO; Evonik) | |
| | Seabuckthorn Pulp Oil (Hippophae Rhamnoides | 0.1 |
| | Fruit Oil (Henry Lamotte Oils GmbH) | |

Recipes 18a: Facial Cleansing Gel

| Ingredients [wt. %] | 18a |
|---|---|
| Water | ad 100 |
| Xanthan Gum (Keltrol CG-SFT) | 1.5 |
| Sodium Cocoamphoacetate (REWOTERIC ® AM C | 10.0 |
| MB; Evonik) | |
| Citric Acid (30% sol.) | 1.6 |
| Capryl/Capramidopropyl Betaine (TEGO ® Betain 810 | 6.6 |
| MB; Evonik) | |
| SFX | 5 |
| Sodium Chloride | q.s |
| Sorbitan Caprylate, Glyceryl Oleate (TEGO ® Remo | 1.5 |
| 95 MB) | |
| Phenoxyethanol; Benzoic Acid (Verstatil ® BP; Evonik) | 0.8 |

Recipes 19a: Wet Wipe solution

| Ingredients [wt. %] | 19a |
|---|---|
| SFX | 8 |
| Aloe Barbadensis Leaf Extract (Aloe-Con UP 40, Florida Food Products Inc.) | 0.2 |
| Isopropyl Myristate (TEGOSOFT ® M, Evonik) | 0.2 |
| Disodium Cocoamphodiacetate (REWOTERIC ® AM 2 C NM, Evonik, 39%) | 1.5 |
| Perfume | 0.2 |
| Propylene Glycol (Euxyl K 320, Schulke & Mayr GmbH) | 2.5 |
| Hydrolyzed Silk (Crosilk 10000, Croda Inc.) | 0.2 |
| Caprylyl/Capryl Glucoside (Plantacare 810 UP, BASF) | 0.7 |
| Water | ad 100.0 |
| Citric Acid | ad pH 5.0 |
| Phenoxyethanol (S&M Phenoxyethanol, Schülke & Mayr GmbH) | 0.5 |
| Dehydroacetic Acid (Unisept DHA (Universal Preserv-A-Chem, Inc.) | 0.1 |
| Sodium Benzoate (Euxyl K 712, Schülke & Mayr GmbH) | 0.4 |
| Salicylic Acid (Salicylic acid nat.; Evonik Dr. Straetmans GmbH) | 0.5 |

Recipes 20a Cleansing Bar

| Ingredients [wt. %] | 20a |
|---|---|
| Sorbitan Oleate (TEGO ® SMO V; Evonik) | 1.00 |
| Theobrom Cacao (Cocoa) Seed Butter | 6.80 |
| Hydrogenated Palm Oil; Hydrogenated Rapeseed Oil | 2.50 |
| (dermofeel ® viscolid MB; Evonik) | |
| Cetearyl Alcohol (TEGO ® Alkanol 1618 MB; Evonik) | 24.5 |
| Sorbitan Caprylate; Glyceryl Oleate (TEGO ® Remo 95 | 3.00 |
| MB; Evonik) | |
| Cellulose (TEGO ® Feel C 10; Evonik) | 2.50 |
| Sodium Coco-Sulfate, 94% | 25.0 |
| Cocamidopropyl Betaine (TEGO ® Betain CK D MB; | 25.0 |
| Evonik) | |
| SFX | 0.8 |
| Tocopherol; Helianthus Annuus (Sunflower) Seed Oil | 0.2 |
| (dermofeel ® Toco 70 non GMO; Evonik) | |
| Glyceryl Caprylate (dermosoft ® GMCY MB; Evonik) | 0.5 |
| Oleyl Erucate (TEGOSOFT ® OER MB; Evonik) | 6.0 |
| Zinc Oxide | 2.0 |
| Perfume | 1.0 |

Recipes 21a: Cleansing Cream

| | Ingredients [wt. %] | 21 |
|---|---|---|
| Phase A | Helianthus Annuus (Sunflower) Seed Oil | 50.0 |
| | Sorbitan Sesquicaprylate (ANTIL ® Soft SC; Evonik) | 7.0 |
| Phase B | Sodium Laureth Sulfate, 28% | 10.7 |
| | SFXs | 3.5 |
| | Glycerin | 1.0 |
| | Water | ad 100 |
| Phase C | Preservative, perfume | q.s. |

Recipes 22a: Body Wash Peeling

| | Ingredients [wt. %] | 22a |
|---|---|---|
| Phase | Helianthus Annuus (Sunflower) Seed Oil | 50.0 |
| A | Diethylhexyl Sodium Sulfosuccinate (TEGO ® | 9.0 |
| | Sulfosuccinate DO 75; Evonik) | |
| Phase | Disodium Laureth Sulfate (REWOPOL ® SB FA 30 | 7.0 |
| | B MB; Evonik) | |
| B | Cocamidopropyl Betaine (TEGO ® Betain F 50 MB; | 5.0 |
| | Evonik) | |
| | SFXs | 4 |
| | Glycerin | 2.0 |
| | Water | ad 100 |
| Phase | Hydrated Silica (Sipernat 2200 PC; Evonik) | q.s. |
| C | Preservative, perfume | q.s. |

Recipes 23a: Cleansing Powder

| | Ingredients [wt. %] | 23a |
|---|---|---|
| Phase | Capryl/Capramidopropyl Betaine (TEGO ® Betain | 12.0 |
| | 810 MB; Evonik) | |
| A | Sodium Cocoamphoacetate (REWOTERIC ® AM C | 15.0 |
| | MB; Evonik) | |
| | Sucrose Cocoate (TEGOSOFT ® LSE 65 K Soft) | 3.0 |
| | Sorbitan Sesquicaprylate (ANTIL ® Soft SC MB; | 1.0 |
| | Evonik) | |
| | Bis-(Isostearoyl/Oleoyl Isopropyl) imonium | 1.0 |
| | Methosulfate (VARISOFT ® EQ 100; Evonik) | |
| | SFXs | 6 |
| | Oleyl Erucate (TEGOSOFT ® OER MB; Evonik) | 2.0 |
| | Helianthus Annuus (Sunflower) Seed Oil | 5.0 |
| | Citric Acid (30% sol.) | 3.0 |
| Phase | Sipernat 500 LS Hydrated Silica) | |
| B | Wheat Flour | 29.0 |
| | Cocamidopropyl Betaine (TEGO ® Betain CK D MB; | 12.5 |
| | Evonik) | |
| | Preservative, Perfume | q.s. |

Recipes 24a: Cleansing Oil

| Ingredients [wt. %] | 24a |
|---|---|
| SFXs | 0.5 |
| Polyglyceryl-2 Sesquioleate (dermofeel ® GO soft; | 5.0 |
| Evonik) | |
| Helianthus Annuus (Sunflower) Seed Oil | 36.0 |
| Isoamyl Laurate (dermofeel ® sensolv MB; Evonik) | 35.0 |
| TEGOSOFT ® DC MB (Decyl Cocoate; Evonik) | 5 |
| Polyglyceryl-2 Caprate (dermosoft ® DGMC MB; | 2 |
| Evonik) | |
| Tocopherol; Helianthus Annuus (Sunflower) Seed Oil | 1.0 |
| (dermofeel ® Toco 70 non GMO; Evonik) | |
| Sesamum Indicum (Sesame Seed) Oil | 16 |

Recipes 25a: Anti-Transpirant Deo

| | Ingredients [wt. %] | 25a |
|---|---|---|
| Phase A | SFX | 1 |
| | Dicaprylyl Ether (Cetiol OE, BASF) | 0.3 |
| | Geraniol (Nerol 800, International Flavors & Fragrances Inc.) | 0.1 |
| | Linalool (Lipofresh, Lipo Chemicals, Inc.) | 0.1 |
| | Perfume | 0.1 |
| Phase B | Propylene Glycol (Euxyl K 320, Schülke & Mayr GmbH) | 1.0 |
| | Butylene Glycol (Oxea Corparation) | 0.2 |
| | Water | 5.0 |
| | Palmitamidopropyltrimonium Chloride (VARISOFT ® PATC, Evonik) | 1.0 |
| Phase C | Water | 50.0 |
| | Hydroxethyl Ethylcellulose (Structure Cel 4400 E, AkzoNobel) | 0.8 |
| | Sodium Hydroxide (10% in water) | 0.3 |
| Phase D | Aluminium Chlorohydrate (Locron L, Clariant) | 15.0 |
| Phase E | Preservative | q.s. |
| | Water | ad 100.0 |

Recipes 26a: Mouthwash

| Ingredients [wt. %] | 26a |
|---|---|
| SFX | 1 |
| Flavor | 0.2 |
| Water | ad 100.0 |
| Sorbitol (Karion FP Liquid, Merck) | 3.0 |
| Preservative | q.s. |
| Dyes | q.s. |

Recipes 27a: Toothpaste

| | Ingredients [wt. %] | 27a |
|---|---|---|
| Phase A | Sorbitol (Karion FP Liquid , Merck) | 50.0 |
| | Water | ad 100.0 |
| | SFX | 2 |
| | Sodium Carboxymethylcellulose (Blanose 7MXF, Ashland) | 1.2 |
| Phase B | Sodium Saccharine (Sigma Aldrich) | 0.1 |
| | Sodium Fluoride (Sigma Aldrich) | 0.1 |
| Phase C | Titanium Dioxide (Caesar & Loretz) | 0.4 |
| | Hydrated Silica (Zeodent ® 113, Evonik) | 14.0 |
| | Hydrated Silica (Zeodent ® 165, Evonik) | 8.0 |
| Phase D | Flavor oil | 1.0 |
| Phase E | Glyceryl Caprylate (dermosoft ® GMCY, Evonik) | 0.3 |

Recipes 28a: Kitchen Cleaning Spray

| Ingredient | 28a |
|---|---|
| REWOTERIC ® AM V | 0.5 |
| SFX | 1 |
| TOMAKLEEN ® G-14 | 2.0 |
| Water (Dye. Perfume) | ad 100 |
| Triethanolamine | 4.0 |

Recipes 29a: Extra Mild Dish Wash Foam

| Ingredient | 29a |
|---|---|
| C10-16 Alkylpolyglucoside. 50 | 10.0 |
| Xanthan Gum | 0.1 |
| Glycerin | 2.0 |
| Water | ad 100 |
| SFX | 0.8 |
| Sophorolipid | 2.0 |
| REWOPOL ® SB CS 50 | 5.0 |
| TEGO ® Betain C 60 | 3.0 |
| Preservative | qs. |

Recipes 30a: Automatic Rinse Aid for Direct Use 1

| Ingredient | 30a |
|---|---|
| TEGOTENS ® EC 11 | 5.0 |
| SFX | 5 |
| Citric acid monohydrate | 5.0 |
| Water (Dye. Perfume) | ad 100 |

Recipes 31a: Automatic Rinse Aid for Direct Use 2

| Ingredient | 31a |
|---|---|
| Citric acid monohydrate | 5.0 |
| Sodium Cumene Sulfonate. 92 | 1.5 |
| Water (Dye; Perfume) | ad 100 |
| TEGOTENS ® EC 11 | 6.0 |
| SFX | 4 |

Recipes 32a: Automatic Rinse Aid for Direct Use 3

| Ingredient | 32a |
|---|---|
| Citric acid monohydrate | 5.0 |
| Sodium Cumene Sulfonate. 92 | 1.5 |
| Water (Dye. Perfume) | ad 100 |
| TEGOTENS ® EC 11 | 7.0 |
| SFX | 3 |

Recipes 33a: Glass Cleaner With
Optimized Antifogging Efficiency

| Ingredient | 33a |
|---|---|
| REWOPOL ® TS 35 | 1.0 |
| SFX | 0.5 |
| Isopropanol | 15.0 |
| EDTA | 1.0 |
| Water (Dye. Perfume) | ad 100 |

Recipes 34a: Oven Cleanser for Smoking Chamber

| Ingredient | 34a |
|---|---|
| Water | ad 100 |
| Trilon A liquid | 8.0 |
| Sodium carbonate | 6.0 |
| REWOTERIC ® AM KSF 40 | 5.0 |
| Laureth-6 | 1.2 |
| SFX | 1.2 |
| DOWANOL DPnB | 1.0 |

Recipes 35a: All Purpose Cleanser (Microemulsion)

| Ingredient | 35a |
|---|---|
| Water | ad 100 |
| TEGOTENS ® AM VSF | 6.0 |
| SFX | 2 |
| REWOPOL ® D 510 NC | 5.0 |
| Undeceth-6 | 3.0 |
| Potassium hydroxide. 50 | 5.0 |
| Chelating Agent. MGDA | 5.0 |

Recipes 36a: Low Foaming Hard Surface Degreaser

| Ingredient | 36a |
|---|---|
| C9-11 FATTY ALCOHOL | 2.0 |
| ETHOXYLATE - 8 MOLES OF EO | |
| SFX | 1 |
| TEGOTENS ® G 826 C | 4.0 |
| Chelating Agent. MGDA | 7.5 |
| Water (Dye. Perfume) | ad 100 |

Recipes 37a: Low Foaming Hard Surface Degreaser (101630-23)

| Ingredient | 37a |
|---|---|
| C9-11 FATTY ALCOHOL | 2.0 |
| ETHOXYLATE - 8 MOLES OF EO | |
| SFX | 1 |
| TEGOTENS ® G 826 C | 4.0 |
| Water (Dye. Perfume) | ad 100 |

Recipes 38a: Foaming Hard Surface Degreaser 1

| Ingredient | 38a |
|---|---|
| C9-11 FATTY ALCOHOL | 2.0 |
| ETHOXYLATE - 8 MOLES OF EO | |
| TEGOTENS ® AM VSF | 6.0 |
| SFX | 1 |
| Water (Dye. Perfume) | ad 100 |

Recipes 39a: Foaming Hard Surface Degreaser 2

| Ingredient | 39a |
|---|---|
| C9-11 FATTY ALCOHOL | 3.0 |
| ETHOXYLATE - 8 MOLES OF EO | |
| REWOTERIC ® AM KSF 40 | 2.5 |
| SFX | 1 |
| Water (Dye. Perfume) | ad 100 |

Recipes 40a: Low Foaming Hard Surface Degreaser 3

| Ingredient | 40a |
|---|---|
| SFX | 0.5 |
| C8-10 Alkylpolyglucosid | 4.0 |
| Sodium C13-17 Alkane Sulfonate. 30 | 2.0 |
| Water (Dye. Perfume) | ad 100 |

Recipes 41a: Low Foaming Hard Surface Degreaser 4

| Ingredient | 41a |
|---|---|
| SFX | 1 |
| C8-10 Alkylpolyglucosid | 4.0 |
| Sodium C13-17 Alkane Sulfonate. 30 | 2.0 |
| Chelating Agent. MGDA | 7.0 |
| Water (Dye. Perfume) | ad 100 |

Recipes 42a: Low Foaming Hard Surface Degreaser 4

| Ingredient | 42a |
|---|---|
| SFX | 1 |
| C8-10 Alkylpolyglucosid | 4.0 |
| Sodium C13-17 Alkane Sulfonate. 30 | 2.0 |
| Water (Dye. Perfume) | ad 100 |

Recipes 43a: Hard Surface Degreaser From Renewable Surfactants

| Ingredient | 43a |
|---|---|
| Water | ad 100 |
| Sodium carbonate | 3.0 |
| TEGOTENS ® AM VSF | 2.4 |
| Laureth-6 | 0.6 |
| SFX | 0.4 |
| TEGO ® Polish Additiv Q 70 | 0.3 |

Recipes 44a: Highly Efficient Floor Cleanser

| Ingredient | 44a |
|---|---|
| Water | ad 100 |
| Desferrioxamine | 4.0 |
| Sodium carbonate | 3.0 |
| TEGOTENS ® AM VSF | 2.4 |
| Laureth-6 | 0.6 |
| SFX | 0.4 |
| TEGO ® Polish Additiv Q 70 | 0.3 |

Recipes 45a: Super Natural Wash Lotion for Textile Face Masks

| Ingredient | 45a |
|---|---|
| Xanthan Gum | 0.1 |
| Glycerin | 2.0 |
| Water | ad 100 |
| SFX | 1 |
| Sophorolipid | 2.0 |
| REWOPOL ® SB CS 50 | 5.0 |
| C8-10 Alkylpolyglucosid | 10.0 |
| TEGO ® Betain C 60 | 3.0 |
| Preservative | qs. |

Recipes 46: Highly Effective Presoaker

| Ingredient | 46a |
|---|---|
| Water | ad 100 |
| Sodium metasilicate | 2.0 |
| Sodium gluconate | 2.0 |
| Sodium carbonate | 2.0 |
| TEGOTENS ® AM VSF | 6.5 |
| Undeceth-6 | 0.6 |
| SFX | 0.4 |
| DOWANOL DPnB | 1.0 |

Recipes 47a: Presoaker (Basic Formula)

| Ingredient | 47a |
|---|---|
| Water | ad 100 |
| Trilon A 92 | 3.0 |
| Sodium metasilicate | 2.0 |
| Sodium carbonate | 2.0 |
| Bayhibit AM | 1.0 |
| REWOTERIC ® AM KSF 40 | 4.0 |
| C9-11 FATTY ALCOHOL ETHOXYLATE - 8 MOLES OF EO | 1.2 |
| SFX | 1 |
| DOWANOL DPnB | 3.0 |

Recipes 48a: Good Dispersing Presoaker

| Ingredient | 48a |
|---|---|
| Water | ad 100 |
| Trilon A 92 | 3.0 |
| Sodium metasilicate | 2.0 |
| Sodium carbonate | 2.0 |
| Sequion 10 Na 430 | 2.0 |
| TEGOTENS ® 475 | 5.0 |
| C9-11 FATTY ALCOHOL ETHOXYLATE - 7 MOLES OF EO | 1.5 |
| SFX | 3 |
| DOWANOL PnB | 3.0 |

Recipes 49a: Cost Efficient Presoaker

| Ingredient | 49a |
|---|---|
| Water | ad 100 |
| Chelating Agent. M | 8.0 |
| Sodium carbonate | 6.0 |
| TEGOTENS ® AM VSF | 3.5 |
| Laureth-6 | 0.6 |
| SFX | 0.4 |
| DOWANOL DPnB | 1.0 |

Recipes 50a: Rinse Aid to minimize remaining water

| Ingredient | 50a |
|---|---|
| REWOPAL ® MPG 40 | 6.0 |
| Butyl Cellusolve (BG) | 14.0 |
| SFX | 3 |
| REWOQUAT ® CR 3099 | 10.0 |
| TEGOSOFT ® OP | 5.0 |
| Water | ad 100 |

Recipes 51a: Rinse Aid with Optimal Oil Content

| Ingredient | 51a |
|---|---|
| DOWANOL DPnB | 8.6 |
| REWOPAL ® MPG 40 | 6.2 |
| REWOQUAT ® CR 3099 | 10.0 |
| REWOCARE ® DOC | 4.0 |
| TEGOSOFT ® OP | 3.0 |
| SFX | 1 |
| Water | ad 100 |
| TEGOPREN ® 6923 | 0.4 |

Recipes 52a: Low Foaming All Purpose Cleaner

| Ingredient | 52a |
|---|---|
| Water | ad 100 |
| Trilon A 92 | 3.0 |
| Sodium metasilicate | 2.0 |
| Sodium carbonate | 2.0 |
| Sequion 10 Na 430 | 2.0 |
| TEGOTENS ® 475 | 5.0 |
| C9-11 FATTY ALCOHOL ETHOXYLATE - 7 MOLES OF EO | 1.5 |
| SFX | 1.5 |
| DOWANOL PnB | 1.0 |

Recipes 53a: Low Foaming Alkaline Cleanser

| Ingredient | 53a |
|---|---|
| Water | ad 100 |
| Chelating Agent. M | 4.0 |
| Sodium carbonate | 6.0 |
| TEGOTENS ® AM VSF | 3.5 |
| Laureth-6 | 0.6 |
| SFX | 1 |

Recipes 54a: Low Foaming Alkaline Cleaner

| Ingredient | 54a |
|---|---|
| Water | ad 100 |
| TOMAKLEEN G-14 | 0.75 |
| TEGOTENS ® AM VSF | 3.5 |
| SFX | 1.8 |
| Sodium carbonate | 6.0 |

Recipes 55a: Alkaline Cleanser (Automatic Wash)

| Ingredient | 55a |
|---|---|
| Water | ad 100 |
| Tetra Potassium Pyrophosphate (TKPP) | 15.0 |
| Trilon A liquid | 5.0 |
| KOH. 45 | 4.0 |
| Sequion 10 Na 430 | 10.0 |
| Sodium metasilicate | 6.0 |
| SFX | 1 |

Recipes 56a: Metal Cleaner

| Ingredient | 56a |
|---|---|
| Water | ad 100 |
| TOMAKLEEN ® G-14 | 12.0 |
| REWOTERIC ® AM V | 1.5 |
| SFX | 3 |
| Triethanolamine | 1.5 |

Recipes 57a: Metal Cleanser

| Ingredient | 67a |
|---|---|
| Water (Dye. Perfume) | ad 100 |
| Triethanolamine | 1.5 |
| DOWANOL PnP | 6.0 |
| REWOTERIC ® AM V | 1.5 |
| SFX | 3 |
| C8-10 Fatty alcohol • 5 EO | 6.0 |

Recipes 58a: Cost Efficient Facade Cleanser

| Ingredient | 58a |
|---|---|
| Water | ad 100 |
| Chelating Agent. MGDA | 4.0 |
| Sodium carbonate | 6.0 |
| TEGOTENS ® AM VSF | 2.4 |
| Laureth-6 | 0.6 |
| SFX | 0.8 |

Recipes 59a: Anti-dust Glass and Window Cleaner 1

| Ingredient | 59a |
|---|---|
| Water | ad 100 |
| Ethanol | 15 |
| Rewocare ® 755 | 0.5 |
| SFX | 2 |
| 2-Phenoxyethanol | 0.1 |
| pH value (adjust to) | 8 |

Recipes 60a: Anti-dust Glass and Window Cleaner 2

| Ingredient | 60a |
|---|---|
| Water | ad 100 |
| Ethanol | 15 |
| Rewocare ® 755 | 0.5 |
| SFX | 1 |
| Tegotens ® AM VSF | 1 |
| 2-Phenoxyethanol | 0.1 |
| pH value (adjust to) | 8.00 |

Recipes 61a: Anti-dust Glass and Window Cleaner 3

| Ingredient | 61a |
|---|---|
| Water | ad 100 |
| Ethanol | 3 |
| SFX | 1.5 |
| Tegotens ® DO 40 | 1 |
| Tegopren ® 5840 | 0.05 |
| 2-Phenoxyethanol | 0.1 |
| pH value (adjust to) | 6.0-7.0 |

Recipes 62a: All-purpose Cleaner concentrate

| Ingredient | 62a |
|---|---|
| Water | ad 100 |
| Ethanol | 3 |
| SFX | 2 |
| Rewocare ® 755 | 0.5 |
| Rewopol ® SB DO 75 | 3 |
| 2-Phenoxyethanol | 0.1 |
| pH value (adjust to) | 8 |

Recipes 63a: All-purpose Cleaner Cleaner Trigger spray

| Ingredient | 63a |
|---|---|
| Water | ad 100 |
| Ethanol | 3 |
| SFX | 1.5 |
| Tego ® Betain Powder MB | 1 |
| Rewocare ® 755 | 0.5 |
| 2-Phenoxyethanol | 0.1 |
| pH value (adjust to) | 8.00 |

Recipes 64a: Floor Cleaner

| Ingredient | 64a |
|---|---|
| Water | ad 100 |
| SFX | 1 |
| Tego ® PP 1027 | 0.5 |
| Tego ® Betain Powder MB | 1 |
| Tomadol 91-6 | 4 |
| 2-Phenoxyethanol | 0.1 |
| pH value (adjust to) | 8.00 |

Recipes 65a: Bathroom Cleaner

| Ingredient | 65a |
|---|---|
| Water | ad 100 |
| SFX | 3 |
| Tego ® Betain Powder MB | 1.5 |
| Rewocare ® 755 | 0.5 |
| Tegotens ® AM VSF | 1.5 |
| 2-Phenoxyethanol | 0.1 |
| pH value (adjust to) | 5.5 |

Recipes 66a, 66b, 66c: Handdishwash Liquid

| Ingredient | 66a | 66b | 66c |
|---|---|---|---|
| Water | ad 100 | ad 100 | ad 100 |
| Texapon 70 | 15 | 5 | |
| TEGO ® Betain C 60 | 4.8 | 3 | 3 |
| SFX | 3 | 2 | 2 |
| Sophorolipid | | 1.3 | |
| Glucolipid | | | 2.6 |
| C10-16 APG, 50 | | 10 | 10 |
| NaCl | 1.25 | | |
| Xanthan Gum | | 0.3 | 0.3 |
| Glycerin | | 2 | 2 |
| Rewopol ® SB CS 50 | | | 5 |
| Preservative | q.s. | q.s. | q.s. |
| pH value | 5.5 | 5.5 | 5.5 |

Recipes 67a: Cleaner for Plastic furniture and interieur

| Ingredient | 67a |
|---|---|
| Water | ad 100 |
| SFX | 1 |
| Tego ® Betain C 60 | 1.5 |
| Tomakleen 91-6 | 1.5 |
| Rewoquat ® Q 70 | 0.5 |
| pH value (adjust to) | 9 |

Recipes 68a: Grill and Oven Cleaner

| Ingredient | 68a |
|---|---|
| Water | ad 100 |
| MGDA | 5 |
| Sodium Carbonate | 5 |
| Rewoteric ® AM KSF 40 MB | 5 |
| Tomadol 91-6 | 1.5 |
| SFX | 1.5 |
| Dowanol DPnB | 1 |
| pH (adjust to) | 12 |

Recipes 69a, 69b, 69c: Cleaner for Food&Beverage Fatsolve

| Ingredient | 69a | 69b | 69c |
|---|---|---|---|
| Water | ad 100 | ad 100 | ad 100 |
| Rewoquat ® CQ 200 | 20 | 12 | 12 |
| Rewoteric ® AM KSF 40 MB | 10 | | |
| SFX | 5 | 5 | 5 |
| MGDA | 5 | | 5 |
| Sodium Carbonate | 3 | | 3 |
| Tomadol 91-8 | | 5 | |
| Tomadol 1-5 | | 3 | 2 |
| pH value (adjust to) | 12 | 12 | 12 |

Recipes 70a, 70b: Car Shampoo

| Ingredient | 70a | 70b |
|---|---|---|
| Water | ad 100 | ad 100 |
| Tomakleen G-14 | 5 | |
| MGDA | 2 | 3 |
| Sodium Carbonate | 2 | |
| SFX | 0.5 | 3 |
| Sophorolipid | 1.5 | 1.5 |
| Tomadol 91-6 | 3 | |
| Rewocare ® 755 | 0.5 | 0.5 |
| Tego ® Betain C 60 | 2 | 3 |
| pH value (adjust to) | 10 | 9 |

Recipes 70a, 70b, 70c: Car Rinse

| Ingredient | 70a | 70b | 70c |
|---|---|---|---|
| Water | ad 100 | ad 100 | ad 100 |
| Carspray 90 MB | 20 | — | — |
| Rewopol ® MPG 40 | — | 5 | 5 |
| SFX | 0.5 | 0.5 | 1 |
| Rewoquat ® CR 3099 | — | 15 | — |
| Tegopren ® 6922 | — | — | 20 |
| Isopropanol | 2 | 2 | 2 |
| pH value (adjust to) | 8 | 8 | 8 |

Recipes 71a, 71b, Automatic Dish Wash Powder

| Ingredient | 71a | 71b |
|---|---|---|
| Sodium carbonate | 42.0 | 42.0 |
| Sodium sulphate | 20.0 | 20.0 |
| Sodium citrate | 15.0 | 15.0 |
| Sodium percarbonate | 10.0 | 10.0 |
| Polymer | 3.0 | 3.0 |
| Phosphonate (HEDP) | 2.5 | 2.5 |
| Sodium metasilicate | 2.0 | 2.0 |
| Tomakleen G-14 | 1.0 | 1.0 |
| SFX | 1 | 1 |
| TAED | 2.0 | 2.0 |
| Protease | 1.0 | — |
| Amylase | 0.5 | — |

Recipes 72a: Automatic Dish Wash Gel

| Ingredient | 72a |
|---|---|
| Water | ad 100 |
| Tetrasodium Glutamare diacetate/GLDA | 14 |
| Glycerol | 9.0 |
| Polyacrylate polymer | 2.0 |
| Protease | 1.0 |
| Formic acid | 0.2 |
| PPG-1 C6-10 Pareth-20 | 2.0 |
| Amylase | 0.50 |
| SFX | 0.5 |
| Xanthan Gum | 0.50 |
| Perfumes | 0.20 |
| Zinc acetate dihydrate | 0.10 |
| Calcium Chloride | 0.10 |
| Limonen | 0.050 |
| Colorant | 0.050 |
| CIT/MIT | 0.050 |

Recipes 73a, 73b, 73c, 73d, 73e, 73f: Laundry pre-spotter

| Ingredient | 73a | 73b | 73c | 73d | 73e | 73f |
|---|---|---|---|---|---|---|
| SFXs | 5 | 5 | 12 | 12 | 5 | 5 |
| Sophorolipids | 6 | 6 | 2.5 | 2.5 | 0.5 | 0.5 |
| Lauryl/Myristil | | | | | 12 | 12 |
| Glucoside | | | | | | |
| Propylene Glycol | 2 | 2 | 2 | 2 | 2 | 2 |
| Na citrate | 2 | 2 | 2 | 2 | 2 | 2 |
| Na formate | 0.5 | 0.5 | 0.5 | 0.5 | 0.5 | 0.5 |
| CaCl2 | 0.1 | 0.1 | 0.1 | 0.1 | 0.1 | 0.1 |
| NaOH | 0.05 | 0.05 | 0.05 | 0.05 | 0.05 | 0.05 |
| Protease | 1 | | 1 | | | 0.4 |
| Amylase | 0.4 | 0.4 | 0.4 | 0.4 | | 0.2 |
| Mannanase | 0.2 | 0.2 | 0.2 | 0.2 | | 0.2 |
| Pectinase | 0.2 | 0.2 | 0.2 | 0.2 | | 0.2 |
| Cellulase | 0.2 | 0.2 | 0.2 | 0.2 | | 0.2 |
| Water | ad 100 | ad 100 | ad 100 | ad 100 | ad 100 | ad 100 |

Recipes 74a, 74, 74c, 74d: Laundry pre-spotter

| Ingredient | 74a | 74b | 74c | 74d |
|---|---|---|---|---|
| SFXs | 5 | 5 | 3 | 3 |
| Lauryl/Myristil | 12 | 12 | 9 | 9 |
| Glucoside | | | | |
| Propylene Glycol | 2 | 2 | 2 | 2 |
| Na citrate | 2 | 2 | 2 | 2 |
| Na formate | 0.5 | 0.5 | 0.5 | 0.5 |
| CaCl2 | 0.1 | 0.1 | 0.1 | 0.1 |
| NaOH | 0.05 | 0.05 | 0.05 | 0.05 |
| Protease | 1 | — | 1 | — |
| Amylase | 0.4 | — | 0.4 | — |
| Mannanase | 0.2 | — | 0.2 | — |
| Pectinase | 0.2 | — | 0.2 | — |
| Cellulase | 0.2 | — | 0.2 | — |
| Water | ad 100 | ad 100 | ad 100 | ad 100 |

Recipes 75a, 75, 75c, 75d: Laundry pre-spotter

| Ingredient | 75a | 75b | 75c | 75d |
|---|---|---|---|---|
| SFXs | 5 | 5 | 1 | 1 |
| Propylene Glycol | 2 | 2 | 2 | 2 |
| Na citrate | 2 | 2 | 2 | 2 |
| Na formate | 0.5 | 0.5 | 0.5 | 0.5 |
| CaCl2 | 0.1 | 0.1 | 0.1 | 0.1 |
| NaOH | 0.05 | 0.05 | 0.05 | 0.05 |
| Protease | 1 | 1 | 1 | 1 |
| Amylase | 0.4 | 0.4 | 0.4 | 0.4 |
| Mannanase | 0.2 | 0.2 | 0.2 | 0.2 |
| Pectinase | 0.2 | 0.2 | 0.2 | 0.2 |
| Cellulase | 0.2 | 0.2 | 0.2 | 0.2 |
| Water | ad 100 | ad 100 | ad 100 | ad 100 |

Recipes 76a, 76b, 76c, 76d, 76e, 76f: laundry detergents

| Ingredient | 76a | 76b | 76c | 76d | 76e | 76f |
|---|---|---|---|---|---|---|
| SFXs | 14 | 7 | 7 | 7 | 7 | 7 |
| C12/14 7EO (Dehydol ® LT7) | — | 5.0 | 5.0 | 2.5 | — | 5.0 |
| SLES 2EO (Texapon ® N70) | — | 7.0 | 5.0 | 2.5 | 5.0 | .0 |
| Linear alkylbenzene Sulfonates (LAS) | — | — | 0.5 | 5 | — | — |
| C8C16 Alkyl polyglucosid | — | — | — | — | 2.5 | — |
| (Glucopon ® 650 EC) | | | | | | |
| Amine oxide | — | — | — | 1 | — | — |
| Sodium xylene sulfonate (Eltesol ® | 1.0 | 1.0 | 1.0 | 1.0 | 1.0 | 1.0 |
| SX40) | | | | | | |
| Na soap from palm kernel oil | 5.00 | 5.00 | 5.00 | 5.00 | 5.00 | 5.00 |
| Amide polyglycol ether (Amidet ® N) | — | — | — | — | — | 1.0 |
| Polyethyleneimine PEI, 20 EO | 1.0 | 1.0 | 1.0 | 1.0 | 1.0 | 1.0 |
| (Sokalan ® HP20) | | | | | | |
| Sodium citrate | 3.50 | 3.50 | 3.50 | 3.50 | 3.50 | 3.50 |
| Sodium Phosphonate (Dequest ® | 2.0 | 2.0 | 2.0 | 2.0 | 2.0 | 2.0 |
| 2066) | | | | | | |
| Ethanol | 2.50 | 2.50 | 2.50 | 2.50 | 2.50 | 2.50 |
| MPG | 1.0 | 1.0 | 1.0 | 1.0 | 1.0 | 1.0 |
| Boric acid | 1.0 | 1.0 | 1.0 | 1.0 | 1.0 | 1.0 |
| Phenoxyethanol (Acticide ® SR150) | 0.45 | 0.45 | 0.45 | 0.45 | 0.45 | 0.45 |
| Liquanase ® 3.5 L | 1.0 | 1.0 | 1.0 | 1.0 | 1.0 | 1.0 |
| Amplify ® Prime 100 L | 0.4 | 0.4 | 0.4 | 0.4 | 0.4 | 0.4 |
| Mannaway ® 200 L | 0.2 | 0.2 | 0.2 | 0.2 | 0.2 | 0.2 |
| Carezyme ® Premium 4500 L | 0.2 | 0.2 | 0.2 | 0.2 | 0.2 | 0.2 |
| XPect ® 1000 L | 0.2 | 0.2 | 0.2 | 0.2 | 0.2 | 0.2 |
| Celluclean ® 5000 L | 0.2 | 0.2 | 0.2 | 0.2 | 0.2 | 0.2 |
| Pristine ® 100 L | 0.1 | 0.1 | 0.1 | 0.1 | 0.1 | 0.1 |
| NaOH | 1.0 | 1.0 | 1.0 | 1.0 | 1.0 | 1.0 |
| Water | ad 100 | ad 100 | ad 100 | ad 100 | ad 100 | ad 100 |
| pH | 8.4 | 8.4 | 8.4 | 8.4 | 8.4 | 8.4 |

Recipes 77a, 77b, 77c, 77d, 77e, 77f: laundry detergents

| Ingredient | 77a | 77b | 77c | 77d | 77e | 77f |
|---|---|---|---|---|---|---|
| SFXs | 15 | 8 | 8 | 8 | 8 | 8 |
| C12/14 7EO (Dehydol ® LT7) | — | 5.0 | 5.0 | 2.5 | | 5.0 |
| SLES 2EO (Texapon ® N70) | — | 7.0 | 5.0 | 2.5 | 5.0 | 5.0 |
| Linear alkylbenzene Sulfonates (LAS) | — | — | 2.5 | 2.5 | — | — |
| C8C 16 Alkyl polyglucosid | — | — | — | — | 2.5 | — |
| (Glucopon ® 650 EC) | | | | | | |
| Amine oxide | — | — | — | 1 | — | — |
| Sodium xylene sulfonate (Eltesol ® SX40) | 1.0 | 1.0 | 1.0 | 1.0 | 1.0 | 1.0 |
| Na soap from palm kernel oil | 5.0 | 5.0 | 5.0 | 5.0 | 5.0 | 5.0 |
| Amide polyglycol ether (Amidet ® N) | — | — | — | — | — | 1.0 |
| Vinylpyrrolidone/Vinylimidazole copolymer | 2.0 | 2.0 | 2.0 | 2.0 | 2.0 | 2.0 |
| (Sokalan ® HP 56) | | | | | | |
| Sodium citrate | 3.5 | 3.5 | 3.5 | 3.5 | 3.5 | 3.5 |
| Sodium Phosphonate (Dequest ® 2066) | 2.0 | 2.0 | 2.0 | 2.0 | 2.0 | 2.0 |
| Ethanol | 2.5 | 2.5 | 2.5 | 2.5 | 2.5 | 2.5 |
| MPG | 1.0 | 1.0 | 1.0 | 1.0 | 1.0 | 1.0 |
| Boric acid | 1.0 | 1.0 | 1.0 | 1.0 | 1.0 | 1.0 |
| Phenoxyethanol (Acticide ® SR150) | 0.45 | 0.45 | 0.45 | 0.45 | 0.45 | 0.45 |
| Liquanase ® 3.5 L | 1.0 | 1.0 | 1.0 | 1.0 | 1.0 | 1.0 |
| Amplify ® Prime 100 L | 0.4 | 0.4 | 0.4 | 0.4 | 0.4 | 0.4 |
| Mannaway ® 200 L | 0.2 | 0.2 | 0.2 | 0.2 | 0.2 | 0.2 |
| Carezyme ® Premium 4500 L | 0.2 | 0.2 | 0.2 | 0.2 | 0.2 | 0.2 |
| XPect ® 1000 L | 0.2 | 0.2 | 0.2 | 0.2 | 0.2 | 0.2 |
| Celluclean ® 5000 L | 0.2 | 0.2 | 0.2 | 0.2 | 0.2 | 0.2 |
| Pristine ® 100 L | 0.1 | 0.1 | 0.1 | 0.1 | 0.1 | 0.1 |
| NaOH | 1.0 | 1.0 | 1.0 | 1.0 | 1.0 | 1.0 |
| Water | ad | ad | ad | ad | ad | ad |
| | 100 | 100 | 100 | 100 | 100 | 100 |
| pH | 8.4 | 8.4 | 8.4 | 8.4 | 8.4 | 8.4 |

Recipes 78a, 78b, 78c, 78d, 78e, 78f: laundry detergents

| Ingredient | 78a | 78b | 78c | 78d | 78e | 78f |
|---|---|---|---|---|---|---|
| SFXs | 15 | 8 | 8 | 8 | 8 | 8 |
| C12/14 7EO (Dehydol ® LT7) | — | 5.0 | 5.0 | 2.5 | — | 5.0 |
| SLES 2EO (Texapon ® N70) | — | 7.0 | 5.0 | 2.5 | 5.0 | 5.0 |
| Linear alkylbenzene Sulfonates (LAS) | — | — | 5 | 5 | — | — |
| C8C 16 Alkyl polyglucosid | — | — | — | — | 2.5 | — |
| (Glucopon ® 650 EC) | | | | | | |
| Sodium xylene sulfonate (Eltesol ® | 1.0 | 1.0 | 1.0 | 1.0 | 1.0 | 1.0 |
| SX40) | | | | | | |
| Na soap from palm kernel oil | 5.0 | 5.0 | 5.0 | 5.0 | 5.0 | 5.0 |
| Amide polyglycol ether (Amidet ® N) | — | — | — | — | — | 1.0 |
| Texcare ® SRN 170 | 2.0 | 2.0 | 2.0 | 2.0 | 2.0 | 2.0 |
| Sodium citrate | 3.5 | 3.5 | 3.5 | 3.5 | 3.5 | 3.5 |
| Sodium Phosphonate (Dequest ® | 2.0 | 2.0 | 2.0 | 2.0 | 2.0 | 2.0 |
| 2066) | | | | | | |
| Ethanol | 2.5 | 2.5 | 2.5 | 2.5 | 2.5 | 2.5 |
| MPG | 1.0 | 1.0 | 1.0 | 1.0 | 1.0 | 1.0 |
| Boric acid | 1.0 | 1.0 | 1.0 | 1.0 | 1.0 | 1.0 |
| Phenoxyethanol (Acticide ® SR150) | 0.45 | 0.45 | 0.45 | 0.45 | 0.45 | 0.45 |
| Liquanase ® 3.5 L | 1.0 | 1.0 | 1.0 | 1.0 | 1.0 | 1.0 |
| Amplify ® Prime 100 L | 0.4 | 0.4 | 0.4 | 0.4 | 0.4 | 0.4 |
| Mannaway ® 200 L | 0.2 | 0.2 | 0.2 | 0.2 | 0.2 | 0.2 |
| Carezyme ® Premium 4500 L | 0.2 | 0.2 | 0.2 | 0.2 | 0.2 | 0.2 |
| XPect ® 1000 L | 0.2 | 0.2 | 0.2 | 0.2 | 0.2 | 0.2 |
| Celluclean ® 5000 L | 0.2 | 0.2 | 0.2 | 0.2 | 0.2 | 0.2 |
| Pristine ® 100 L | 0.1 | 0.1 | 0.1 | 0.1 | 0.1 | 0.1 |
| NaOH | 1.0 | 1.0 | 1.0 | 1.0 | 1.0 | 1.0 |
| Water | ad 100 | ad 100 | ad 100 | ad 100 | ad 100 | ad 100 |
| pH | 8.4 | 8.4 | 8.4 | 8.4 | 8.4 | 8.4 |

Recipes 79a, 79b, 79c, 79d, 79e, 79f: laundry detergents

| Ingredient | 79a | 79b | 79c | 79d | 79e | 79f |
|---|---|---|---|---|---|---|
| SFXs | 15 | 8 | 8 | 8 | 8 | 8 |
| C12/14 7EO (Dehydol ® LT7) | — | 5.0 | 5.0 | 2.5 | — | 5.0 |
| SLES 2EO (Texapon ® N70) | — | 7.0 | 5.0 | 2.5 | 5.0 | 5.0 |
| Linear alkylbenzene Sulfonates (LAS) | — | — | 5 | 5 | — | — |
| C8C 16 Alkyl polyglucosid | — | — | — | — | 2.5 | — |
| (Glucopon ® 650 EC) | | | | | | |
| Amine oxide | — | — | — | 1 | — | — |
| Sodium xylene sulfonate (Eltesol ® | 1.0 | 1.0 | 1.0 | 1.0 | 1.0 | 1.0 |
| SX40) | | | | | | |
| Na soap from palm kernel oil | 5.0 | 5.0 | 5.0 | 5.0 | 5.0 | 5.0 |
| Amide polyglycol ether (Amidet ® N) | — | — | — | — | — | 1.0 |
| Texcare ® SRN 170 | 2.0 | 2.0 | 2.0 | 2.0 | 2.0 | 2.0 |
| Sodium citrate | 3.5 | 3.5 | 3.5 | 3.5 | 3.5 | 3.5 |
| Sodium Phosphonate (Dequest ® | 2.0 | 2.0 | 2.0 | 2.0 | 2.0 | 2.0 |
| 2066) | | | | | | |
| Ethanol | 2.5 | 2.5 | 2.5 | 2.5 | 2.5 | 2.5 |
| MPG | 1.0 | 1.0 | 1.0 | 1.0 | 1.0 | 1.0 |
| Phenoxyethanol (Acticide ® SR150) | 0.45 | 0.45 | 0.45 | 0.45 | 0.45 | 0.45 |
| Liquanase ® 3.5 L | 1.0 | 1.0 | 1.0 | 1.0 | 1.0 | 1.0 |
| Amplify ® Prime 100 L | 0.4 | 0.4 | 0.4 | 0.4 | 0.4 | 0.4 |
| Mannaway ® 200 L | 0.2 | 0.2 | 0.2 | 0.2 | 0.2 | 0.2 |
| Carezyme ® Premium 4500 L | 0.2 | 0.2 | 0.2 | 0.2 | 0.2 | 0.2 |
| XPect ® 1000 L | 0.2 | 0.2 | 0.2 | 0.2 | 0.2 | 0.2 |
| Celluclean ® 5000 L | 0.2 | 0.2 | 0.2 | 0.2 | 0.2 | 0.2 |
| Pristine ® 100 L | 0.1 | 0.1 | 0.1 | 0.1 | 0.1 | 0.1 |
| NaOH | 1.0 | 1.0 | 1.0 | 1.0 | 1.0 | 1.0 |
| Water | ad 100 | ad 100 | ad 100 | ad 100 | ad 100 | ad 100 |
| pH | 8.4 | 8.4 | 8.4 | 8.4 | 8.4 | 8.4 |

Recipes 80a, 80b, 80c, 80d, 80e, 80f: laundry detergents

| Ingredient | 80a | 80b | 80c | 80d | 80e | 80f |
|---|---|---|---|---|---|---|
| SFXs | 14 | 8 | 8 | 8 | 8 | 8 |
| C12/14 7EO (Dehydol ® LT7) | — | 5.0 | 5.0 | 2.5 | | 5.0 |
| SLES 2EO (Texapon ® N70) | — | 7.0 | 5.0 | 2.5 | 5.0 | 5.0 |
| Linear alkylbenzene Sulfonates | — | — | 0.5 | 5 | — | — |
| (LAS) | | | | | | |
| Sodium xylene sulfonate | 1.0 | 1.0 | 1.0 | 1.0 | 1.0 | 1.0 |
| (Eltesol ® SX40) | | | | | | |
| Na soap from palm kernel oil | 5.00 | 5.00 | 5.00 | 5.00 | 5.00 | 5.00 |
| Texcare ® SRN 170 | 1 | 1 | 1 | 1 | 1 | — |
| Sodium citrate | 6 | 6 | 6 | 6 | 6 | 6 |
| Phenoxyethanol (Acticide ® SR150) | 0.45 | 0.45 | 0.45 | 0.45 | 0.45 | 0.45 |
| Liquanase ® 3.5 L | 1.0 | 1.0 | 1.0 | 1.0 | 1.0 | 1.0 |
| Amplify ® Prime 100 L | 0.4 | 0.4 | 0.4 | 0.4 | 0.4 | 0.4 |
| Mannaway ® 200 L | 0.2 | 0.2 | 0.2 | 0.2 | 0.2 | 0.2 |
| Carezyme ® Premium 4500 L | 0.2 | 0.2 | 0.2 | 0.2 | 0.2 | 0.2 |
| XPect ® 1000 L | 0.2 | 0.2 | 0.2 | 0.2 | 0.2 | 0.2 |
| Celluclean ® 5000 L | 0.2 | 0.2 | 0.2 | 0.2 | 0.2 | 0.2 |
| Pristine ® 100 L | 0.1 | 0.1 | 0.1 | 0.1 | 0.1 | 0.1 |
| NaOH | 1.0 | 1.0 | 1.0 | 1.0 | 1.0 | 1.0 |
| Desferrioxamine | 1.0 | 1.0 | 1.0 | 1.0 | 1.0 | 1.0 |
| Water | ad 100 | ad 100 | ad 100 | ad 100 | ad 100 | ad 100 |
| pH | 8.4 | 8.4 | 8.4 | 8.4 | 8.4 | 8.4 |

### Shampoo Recipes:

Recipes 81: Volume and Body Shampoo

| Ingredient | % |
|---|---|
| Aqua | ad 100 |
| Sodium Lauryl Sulfate | 7.0 |
| Sodium Laureth Sulfate | 5.0 |
| Cocamidopropyl Betaine | 2.0 |
| SFX | 2.0 |
| Sodium Citrate | 1.5 |
| Sodium Xylenesulfonate | 1.5 |
| Sodium Chloride | 1.4 |
| Citric Acid | ad pH 5.5 |
| Sodium Levulinate, | 1.0 |
| Sodium Benzoate | |
| Hydroxypropyl Methylcellulose | 1.0 |
| Tetrasodium EDTA | 0.8 |
| Butylphenyl Methylpropional | 0.5 |
| Panthenol | 0.5 |
| Panthenyl Ethyl Ether | 0.3 |
| Starch Hydroxypropltrimonium | 0.2 |
| chloride | |
| Linalool | 0.1 |
| Hexyl Cinnamal | 0.1 |
| Limonene | 0.1 |
| Benzyl Salicylate | 0.2 |
| Magnesium Nitrate | 0.1 |
| Magnesium Chloride | 0.1 |
| Methylchloroisothiazolinone | 0.1 |
| Dyes | q.s. |

Recipes 82: Repair Shampoo

| Ingredient | % |
|---|---|
| Aqua | ad 100 |
| Sodium Laureth Sulfate | 7.0 |
| SFX | 3.0 |
| Glycol Distearate | 2.5 |
| Cocamidopropyl Betaine | 2.0 |
| Sodium Citrate | 1.8 |
| Cocamide MEA | 1.8 |
| Sodium Xylenesulfonate | 1.5 |
| Dimethicone | 1.5 |
| Citric Acid | ad pH 5.0 |
| Sodium Benzoate | 0.7 |
| Polyquaternium-76 | 0.5 |
| Sodium Chloride | 0.9 |
| Glycerin | 0.5 |
| Tetrasodium EDTA | 0.3 |
| Butylphenyl Methylpropional | 0.2 |
| Hexyl Cinnamal | 0.1 |
| Linalool | 0.1 |
| alpha-Isomethyl Ionone | 0.1 |
| Geraniol | 0.2 |
| Hydrochloric Acid | 0.1 |
| Magnesium Nitrate | 0.1 |
| Methylchloroisothiazolinone | 0.1 |
| Magnesium Chloride | 0.1 |
| Methylisothiazolinone | 0.1 |
| Dyes | q.s. |

Recipes 83: Anti-Dandruff Shampoo

| Ingredient | % |
|---|---|
| Aqua | ad 100 |
| Sodium Laureth Sulfate | 6.0 |
| Sodium Lauryl Sulfate | 5.0 |
| SFX | 4.0 |
| Cocamide MEA | 4.0 |
| Zinc Carbonate | 3.0 |
| Glycol Distearate | 2.5 |
| Dimethicone | 1.0 |
| Zinc Pyrithione | 1.0 |
| Sodium Chloride | 0.6 |
| Cetyl Alcohol | 0.8 |
| Guar Hydroxypropyltrimonium | 0.3 |
| Chloride | |
| Sodium Xylenesulfonate | 0.5 |
| Magnesium Sulfate | 0.7 |
| Sodium Benzoate | 0.7 |
| Ammonium Laureth Sulfate | 0.3 |
| Citric Acid | ad pH 4.5 |
| Benzyl Alcohol | 0.2 |
| Methylchloroisothiazolinone | 0.1 |
| Methylisothiazolinone | 0.1 |
| Parfum, Dyes | q.s. |

Recipes 84: Strengthening Shampoo

| Ingredient | % |
|---|---|
| Water | ad 100 |
| Sodium Lauryl Sulfate | 6.0 |
| Sodium Laureth Sulfate | 4.0 |
| Cocamidopropyl Betaine | 3.0 |
| SFX | 3.0 |
| Glycol Distearate | 2.5 |
| Dimethicone | 2.0 |
| Sodium Citrate | 1.5 |
| Cocamide MEA | 1.0 |
| Sodium Xylenesulfonate | 1.0 |
| Citric Acid | ad pH 5.0 |
| Sodium Benzoate | 0.7 |
| Sodium Chloride | 0.8 |
| Tetrasodium EDTA | 0.3 |
| Polyquaternium-6 | 0.5 |
| Honey (Mel) | 0.5 |
| Prunus Armeniaca (Apricot) | 0.5 |
| Fruit Extract | |
| Methylchloroisothiazolinone | 0.1 |
| Methylisothiazolinone | 0.1 |
| Parfum, Dyes | q.s. |

Recipes 85: Shampoo

| Ingredient | % |
|---|---|
| Aqua | ad 100 |
| Sodium Laureth Sulfate | 9.0 |
| Cocamidopropyl Betaine | 3.0 |
| SFX | 3.0 |
| Glycerin | 2.5 |
| Sodium Chloride | 1.7 |
| Glycol Distearate | 1.5 |
| Panthenol | 0.2 |
| Propylene Glycol | 0.3 |
| Hydroxypropyl Guar | 0.3 |
| Hydroxypropyltrimonium Chloride | |
| Sodium Benzoate | 0.7 |
| PEG-55 Propylene Glycol Oleate | 0.5 |
| Citric Acid | ad pH 5.5 |
| Hydrolyzed Wheat Protein | 0.3 |
| Argania Spinosa Kernel Oil | 0.1 |
| Laureth-4 | 0.5 |
| Potassium Sorbate | 0.2 |
| Parfum, Dyes | q.s. |

Recipes 86: Moisturising Shampoo

| Ingredient | % |
|---|---|
| Aqua | ad 100 |
| Sodium Laureth Sulfate | 8.0 |
| Cocamidopropyl Betaine | 3.0 |
| Sodium Chloride | 2.2 |
| SFX | 2.0 |
| Hydrolyzed Keratin | 2.0 |
| Water Lily | 1.8 |
| Nymphaea Odorata Root Extract | 1.5 |
| Glyceryl Glucoside | 1.0 |
| Panthenol | 1.0 |
| Decyl Glucoside | 1.0 |
| Polyquaternium-10 | 0.2 |
| Guar Hydroxypropyltrimonium | 0.3 |
| Chloride | |
| PEG-40 Hydrogenated Castor Oil | 0.7 |
| Glycerin | 0.5 |
| Citric Acid | ad pH 5.0 |
| Sodium Benzoate | 1.2 |
| Propylene Glycol | 1.0 |
| Citronellol | 0.1 |
| Limonene | 0.1 |
| alpha-Isomethyl Ionone | 0.1 |
| Benzyl Alcohol | 0.2 |
| Dyes | q.s. |

Recipes 87: Shampoo

| Ingredient | % |
|---|---|
| Aqua | ad 100 |
| Decyl Glucoside | 6.0 |
| Sodium Myreth Sulfate | 5.5 |
| SFX | 3.0 |
| PEG-200 Hydrogenated | 2.5 |
| Glyceryl Palmate | |
| PEG-80 Sorbitan Laurate | 2.0 |
| PEG-90 Glycol Isostearate | 2.0 |
| Disodium PEG-5 Laurylcitrate | 1.5 |
| Sulfosuccinate | |
| Polyquaternium-10 | 1.2 |
| Hydrolyzed Silk | 1.2 |
| Laureth-2 | 1.2 |
| Citric Acid | ad pH 5.5 |
| Sodium Laureth Sulfate | 1.0 |
| PEG-40 Hydrogenated Castor Oil | 0.9 |
| Sodium Benzoate | 0.5 |
| Parfum, Dyes | q.s. |

Recipes 88: Cleansing Oil Shampoo

| Ingredient | % |
|---|---|
| Aqua | ad 100 |
| Sodium Laureth Sulfate | 7.0 |
| MIPA-Laureth Sulfate | 4.0 |
| Sodium Chloride | 3.2 |
| Cocamidopropyl Betaine | 3.0 |
| SFX | 3.0 |
| Glycerin | 2.5 |
| PEG-18 Castor Oil Dioleate | 2.0 |
| Propylene Glycol | 2.0 |
| PEG-55 Propylene Glycol Oleate | 1.7 |
| Behenamidopropyl Dimethylamine | 1.2 |
| Laureth-5 Carboxylic Acid | 1.0 |
| Persea Gratissima (Avocado) Oil | 1.0 |
| PPG-5-Ceteth-20 | 1.0 |
| Sodium Benzoate | 0.7 |
| Laureth-2 | 0.5 |
| Salicylic Acid | 0.3 |
| Linalool | 0.2 |
| alpha-Isomethyl Ionone | 0.1 |
| Limonene | 0.1 |
| Zea Mays (Corn) Germ Oil | 0.2 |
| Argania Spinosa Oil Kernel Oil | 0.1 |
| Camellia Oleifera Seed Oil | 0.1 |
| Sodium Hydroxide | 0.3 |
| Citric Acid | ad pH 5.0 |
| Parfum, Dyes | q.s. |

Recipes 89: Shine Shampoo

| Ingredient | % |
|---|---|
| Aqua | ad 100 |
| Ammonium Lauryl Sulfate | 5.5 |
| SFX | 2.0 |
| Cocamidopropyl Betaine | 2.0 |
| Sodium Chloride | 2.0 |
| Cocamide MEA | 2.0 |
| Nacre Powder | 1.0 |
| Sodium Benzoate | 0.7 |
| Hydroxypropyl Guar | 0.7 |
| Hydroxypropyltrimonium Chloride | |
| Hydroxypropyltrimonium | 0.7 |
| Hydrolyzed Wheat Protein | |
| Sodium Hydroxide | 0.2 |
| Salicylic Acid | 0.5 |
| Limonene | 0.1 |
| Benzoic Acid | 0.5 |
| Linalool | 0.3 |
| Benzyl Alcohol | 0.5 |
| Tin Oxide | 0.2 |
| alpha-Isomethyl Ionone | 0.1 |
| Acrylates Copolymer | 0.1 |
| Citric Acid | ad pH 5.5 |
| Citronellol | 0.1 |
| Hexyl Cinnamal | 0.1 |
| Dyes | q.s. |

Recipes 90: Shampoo

| Ingredient | % |
|---|---|
| Aqua | ad 100 |
| Sodium Cocoyl Isethionate | 3.5 |
| Sodium Lauryl Sulfoacetate | 3.5 |
| Disodium Laureth Sulfosuccinate | 3.5 |
| Cocamidopropyl Betaine | 3.0 |
| SFX | 3.0 |
| Behenamidopropyl Dimethylamine | 0.9 |
| Sodium Lauroyl Sarcosinate | 1.0 |
| Glycol Distearate | 1.0 |
| C11-15 Pareth-7 | 1.0 |
| C12-13 Pareth-23 | 1.0 |
| C12-13 Pareth-3 | 1.0 |
| Decyl Glucoside | 1.0 |
| Tocopherol | 1.0 |
| Hydrogenated Coconut Acid | 1.0 |
| Sodium Chloride | 0.8 |
| Sodium Isethionate | 0.7 |
| Sodium Benzoate | 0.7 |
| Sodium Hydroxide | 0.3 |
| Phenoxyethanol | 0.6 |
| PPG-5 Ceteth-20 | 0.5 |
| PEG-55 Propylene Glycol Oleate | 0.5 |
| Trideceth-12 | 0.5 |
| Polyquaternium-7 | 0.5 |
| Polyquaternium-10 | 0.3 |
| Limonene | 0.1 |
| Benzoic Acid | 0.3 |
| Benzophenone-4 | 0.3 |
| Benzyl Salicylate | 0.2 |
| Linalool | 0.6 |
| Benzyl Alcohol | 0.6 |
| Amodimethicone | 0.5 |
| Propylene Glycol | 0.5 |
| Carbomer | 0.7 |
| Rosmarinus Officinalis | 0.7 |
| (Rosemary) Leaf Oil | |
| Methylparaben | 0.3 |
| Methylchloroisothiazolinone | 0.1 |
| Methylisothiazolinone | 0.1 |
| Citric Acid | ad pH 5.0 |
| Laureth-9 | 0.5 |
| Divinyldimethicone/Dimethicone | 0.2 |
| Copolymer | |
| Glycerin | 0.2 |
| Parfum, Dyes | q.s. |

Recipes 91: Reinforcing Shampoo

| Ingredient | % |
|---|---|
| Aqua | ad 100 |
| Sodium Laureth Sulfate | 6.0 |
| Dimethicone | 3.0 |
| Sodium Chloride | 3.0 |
| Coco-Betaine | 3.0 |
| SFX | 2.0 |
| Glycol Distearate | 1.0 |
| Guar Hydroxypropyltrimonium | 0.3 |
| Chloride | |
| Cocamide MIPA | 0.5 |
| Hydrolyzed keratin | 0.7 |
| Sodium Benzoate | 0.7 |
| Hydroxypropyltrimonium | 0.3 |
| Hydrolyzed Wheat Protein | |
| Sodium Cocoate | 0.7 |
| Sodium Hydroxide | 0.6 |
| Arginine | 0.7 |
| Salicylic Acid | 0.5 |
| Limonene | 0.2 |
| Linalool | 0.2 |
| 2-Oleamido-1.3-Octadecanediol | 0.1 |
| Carbomer | 0.5 |
| Methyl Cocoate | 0.5 |
| Citric Acid | ad pH 5.0 |
| Hexylene Glycol | 0.2 |
| Hexyl Cinnamal | 0.1 |
| Glyceryl Linoleate | 0.1 |
| Glyceryl Oleate | 0.2 |
| Glyceryl Linolenate | 0.2 |
| Dyes | q.s. |

Recipes 92: Shampoo

| Ingredient | % |
|---|---|
| Aqua | ad 100 |
| Sodium Laureth Sulfate | 9.0 |
| Dimethicone | 3.5 |
| Sodium Chloride | 3.2 |
| Coco-Betaine | 3.0 |
| Glycol Distearate | 3.0 |
| SFX | 2.0 |
| Guar Hydroxypropyltrimonium Chloride | 0.3 |
| Niacinamide | 0.1 |
| Cocamide MIPA | 2.0 |
| Saccharum Officinarum | 2.0 |
| (Sugar Cane) Extract | |
| Sodium Benzoate | 1.0 |
| Sodium Cocoate | 1.5 |
| Sodium Hydroxide | 0.6 |
| Salicylic Acid | 0.5 |
| Camellia Sinensis Extract/ | 0.5 |
| Camellia Sinensis Leaf Extract | |
| Linalool | 0.2 |
| Benzyl Salicylate | 0.2 |
| Pyrus Malus (Apple) Fruit Extract | 0.3 |
| Carbomer | 0.5 |
| Pyridoxine HCl | 0.3 |
| Persea Gratissima (Avocado) Oil | 0.2 |
| Citric Acid | ad pH 5.5 |
| Butylphenyl Methylpropional | 0.1 |
| Methyl Cocoate | 0.3 |
| Hexylene Glycol | 0.1 |
| Hexyl Cinnamal | 0.1 |
| Dyes | q.s. |

Recipes 93: Fortifying Shampoo

| Ingredient | % |
|---|---|
| Aqua | ad 100 |
| Ammonium Lauryl Sulfate | 6.0 |
| Cocamidopropyl Betaine | 3.0 |
| Sodium Chloride | 2.7 |
| SFX | 2.0 |
| Niacinamide | 0.1 |
| Saccharum Officinarum | 0.9 |
| (Sugar Cane) Extract | |
| Cocos Nucifera (Coconut) Oil | 0.9 |
| Sodium Benzoate | 0.9 |
| Hydroxypropyl Guar | 0.1 |
| Hydroxypropyltrimonium Chloride | |
| Sodium Hydroxide | ad pH |
| Salicylic Acid | 0.4 |
| Limonene | 0.1 |
| Benzoic Acid | 0.4 |
| Linalool | 0.3 |
| Cinnamyl Alcohol | 0.2 |
| Pyrus Malus (Apple) | 0.5 |
| Fruit Extract | |
| Pyridoxine HCl | 0.2 |
| Bisabolol | 0.2 |
| Citric Acid | ad pH 5.0 |
| Citronellol | 0.1 |
| Hexylene Glycol | 0.2 |
| Dyes | q.s. |

Recipes 94: Shampoo

| Ingredient | % |
|---|---|
| Aqua | ad 100 |
| Sodium Laureth Sulfate | 9.0 |
| Cocamidopropyl Betaine | 2.5 |
| SFX | 2.5 |
| Sodium Chloride | 2.5 |
| Camellia Sinensis Leaf Extract | 2.0 |
| Guar Hydroxypropyltrimonium Chloride | 0.3 |
| Maltodextrin | 1.2 |
| Disodium EDTA | 1.0 |
| PPG-12 | 0.7 |
| Citric Acid | ad pH 5.0 |
| Sodium Hydroxide | 0.3 |
| Sodium Benzoate | 0.7 |
| Butylphenyl Methylpropional | 0.2 |
| Geraniol | 0.2 |
| Hexyl Cinnamal | 0.1 |
| Limonene | 0.1 |
| Linalool | 0.1 |
| Dyes | q.s. |

Recipes 95: Brunette Shampoo

| Ingredient | % |
|---|---|
| Aqua | ad 100 |
| Sodium Laureth Sulfate | 8.5 |
| Cocamidopropyl Betaine | 4.0 |
| Sodium Chloride | 3.5 |
| SFX | 2.0 |
| Selaginella Lepidophylla Aerial Extract | 2.0 |
| Gluconolactone | 1.8 |
| Glycerin | 1.8 |
| Dimethiconol | 1.0 |
| Guar Hydroxypropyltrimonium Chloride | 0.5 |
| TEA-Dodecylbenzenesulfonate | 1.0 |
| Maltodextrin | 1.0 |
| Disodium EDTA | 0.7 |
| Carbomer | 0.9 |
| PPG-12 | 0.7 |
| Citric Acid | ad pH 5.5 |
| Sodium Hydroxide | 0.9 |
| TEA-Sulfate | 0.7 |
| Triethanolamine | 0.5 |
| DMDM Hydantoin | 0.1 |
| Sodium Benzoate | 0.7 |
| Methylchloroisothiazolinone | 0.1 |
| Methylisothiazolinone | 0.1 |
| alpha-Isomethyl Ionone | 0.1 |
| Butylphenyl Methylpropional | 0.2 |
| Hexyl Cinnamal | 0.1 |
| Limonene | 0.1 |
| Linalool | 0.1 |
| Dyes | q.s. |

Recipes 96: 2 in 1 Shampoo

| Ingredient | % |
|---|---|
| Aqua | ad 100 |
| Sodium Laureth Sulfate | 8.0 |
| PEG-200 Hydrogenated Glyceryl Palmate | 3.0 |
| SFX | 3.0 |
| Disodium Cocoamphodiacetate | 3.0 |
| Polysorbate 20 | 2.5 |
| Glycerin | 2.5 |
| Sodium Chloride | 2.0 |
| PEG-7 Glyceryl Cocoate | 2.0 |
| Sodium Benzoate | 1.0 |
| Sodium Laureth-8 Sulfate | 1.0 |
| Sodium Oleth Sulfate | 1.0 |
| Sodium Hydroxide | 0.3 |
| PPG-5 Ceteth-20 | 1.0 |
| PEG-55 Propylene Glycol Oleate | 0.5 |
| Polyquaternium-10 | 0.2 |
| Salicylic Acid | 0.3 |
| Maltodextrin | 0.2 |
| Propylene Glycol | 0.2 |
| Disodium Ricinoleamido MEA-Sulfosuccinate | 0.1 |
| Citric Acid | ad pH 5.5 |
| Prunus Armeniaca (Apricot) Kernel Oil | 0.1 |
| Parfum, Dyes | q.s. |

Recipes 97: Shampoo

| Ingredient | % |
|---|---|
| Aqua | ad 100 |
| Sodium Laureth Sulfate | 9.0 |
| Cocamidopropyl Betaine | 3.0 |
| Sodium Chloride | 2.5 |
| Glycol Distearate | 2.5 |
| Hydrolyzed Keratin | 2.0 |
| Simmondsia Chinensis (Jojoba) Seed Oil | 2.0 |
| SFX | 2.0 |
| Argania Spinosa Kernel Oil | 0.5 |
| Persea Gratissima (Avocado) Oil | 0.5 |
| Rosmarinus Officinalis (Rosemary) Leaf Extract | 0.3 |
| Helianthus Annuus (Sunflower) Seed Oil | 0.3 |
| Cocamide MEA | 0.4 |
| Guar Hydroxypropyltrimonium Chloride | 0.2 |
| Polyquaternium-47 | 0.2 |
| PEG-20 Castor Oil | 0.5 |
| Phenethyl Benzoate | 0.7 |
| Disodium EDTA | 0.2 |
| Methylchloroisothiazolinone | 0.3 |
| Methylisothiazolinone | 0.3 |
| Benzyl Salicylate | 0.1 |
| Hexyl Cinnamal | 0.1 |
| Citronellol | 0.1 |
| Dyes | q.s. |

Recipes 98: Foam Shampoo

| Ingredient | % |
|---|---|
| Aqua | ad 100 |
| Sodium Laureth Sulfate | 5.0 |
| Isobutane | 5.0 |
| Decyl Glucoside | 5.0 |
| Propane | 3.5 |
| Sodium Cocoamphoacetate | 3.5 |
| SFX | 3.5 |
| Panthenol | 0.2 |
| Polyquaternium-10 | 0.2 |
| Hydroxypropyl Guar Hydroxypropyltrimonium Chloride | 0.1 |
| Sodium PCA | 0.5 |
| Octyldodecyl PCA | 0.5 |
| Maltodextrin | 0.5 |
| Tilia Cordata Flower Extract | 0.7 |
| Sodium Chloride | 0.9 |
| Citric Acid | ad pH 5.0 |
| Glycerin | 0.9 |
| Propylene Glycol | 0.3 |
| Butane | 0.7 |
| Sodium Acetate | 0.7 |
| Sorbitol | 0.5 |
| Isopropyl Alcohol | 0.5 |
| Dyes | q.s. |

Recipes 99: Anti-Dandruff Shampoo

| Ingredient | % |
|---|---|
| Aqua | ad 100 |
| Sodium Laureth Sulfate | 10.0 |
| Cocamidopropyl Betaine | 3.0 |
| Acrylates Copolymer | 2.5 |
| Sodium Lauroyl Glutamate | 2.5 |
| Zinc Pyrithione | 2.0 |
| SFX | 2.0 |
| Stearamidopropyl Dimethylamine | 1.0 |
| Piroctone Olamine | 0.7 |
| Hydroxypropyl Guar Hydroxypropyltrimonium Chloride | 0.2 |
| Propylene Glycol | 0.5 |
| Sodium PCA | 0.2 |
| Citrus Medica Limonum (Lemon) Peel Extract | 0.5 |
| Citric Acid | ad pH 5.0 |
| Glycol Distearate | 0.8 |
| PPG-9 | 0.2 |
| Laureth-4 | 0.2 |
| Sodium Chloride | 0.9 |
| Sodium Hydroxide | 0.3 |
| Sodium Naphthalenesulfonate | 0.7 |
| Sorbitol | 0.7 |
| DMDM Hydantoin | 0.1 |
| Sodium Benzoate | 0.8 |
| Limonene | 0.1 |
| Linalool | 0.1 |
| Butylphenyl Methylpropional | 0.1 |
| alpha-Isomethyl Ionone | 0.1 |
| Parfum, Dyes | q.s. |

Recipes 100: Shampoo

| Ingredient | % |
|---|---|
| Aqua | ad 100 |
| Sodium Laureth Sulfate | 5.0 |
| Disodium Cocoamphodiacetate | 2.0 |
| SFX | 2.0 |
| Laureth-2 | 0.3 |
| Panthenol | 0.2 |
| Niacinamide | 0.1 |
| Sodium Chloride | 0.7 |
| Citric Acid | ad pH 5.0 |
| PEG-7 Glyceryl Cocoate | 0.5 |
| Sodium Benzoate | 0.5 |
| Polyquaternium-10 | 0.7 |
| Salicylic Acid | 0.5 |
| Hexyl Salicylate | 0.2 |
| Benzyl Salicylate | 0.1 |
| Hexyl Cinnamal | 0.1 |
| Linalool | 0.1 |
| Limonene | 0.1 |
| Propylene Glycol | 0.2 |
| Dyes | q.s. |

Recipes 101: Intensive Care Oil-Shampoo

| Ingredient | % |
|---|---|
| Aqua | ad 100 |
| Sodium Laureth Sulfate | 8.0 |
| Disodium Cocoamphodiacetate | 3.0 |
| SFX | 0.5 |
| Prunus Armeniaca (Apricot) Kernel Oil | 2.5 |
| Hydrolyzed Keratin | 2.5 |
| Panthenol | 2.4 |
| Sodium Chloride | 2.2 |
| Citric Acid | ad pH 5.5 |
| Stearamidopropyl Dimethylamine | 1.2 |
| Polyquaternium-10 | 0.5 |
| Sodium Benzoate | 0.5 |
| PEG-7 Glyceryl Cocoate | 0.7 |
| Laureth-2 | 0.4 |
| PEG-40 Hydrogenated Castor Oil | 0.4 |
| Propylene Glycol | 0.8 |
| PEG-55 Propylene Glycol Oleate | 0.7 |
| Hexyl Cinnamal | 0.1 |
| Butylphenyl Methylpropional | 0.2 |
| Benzyl Salicylate | 0.1 |
| Linalool | 0.2 |
| Benzyl Alcohol | 0.5 |
| Limonene | 0.1 |
| Sodium Acetate | 0.2 |
| Dyes | q.s. |

Recipes 102: Shampoo

| Ingredient | % |
|---|---|
| Aqua | ad 100 |
| Sodium Laureth Sulfate | 8.5 |
| Disodium Cocoamphodiacetate | 3.5 |
| Sodium Chloride | 3.5 |
| SFX | 3.0 |
| Hydrolyzed Collagen | 1.0 |
| Panthenol | 0.4 |
| Cocodimonium Hydroxypropyl Hydrolyzed Keratin | 0.3 |
| Hydrolyzed Keratin | 0.3 |
| Citric Acid | ad pH 5.5 |
| Sodium Benzoate | 0.7 |
| Polyquaternium-10 | 0.2 |
| Hexyl Cinnamal | 0.1 |
| Limonene | 0.1 |
| Linalool | 0.1 |
| Benzyl Salicylate | 0.1 |
| Benzyl Alcohol | 0.2 |
| Propylene Glycol | 0.2 |
| Dyes | q.s. |

Recipes 103: Shampoo

| Ingredient | % |
|---|---|
| Aqua | ad 100 |
| Sodium Laureth Sulfate | 8.0 |
| Cocamidopropyl Betaine | 3.0 |
| SFX | 3.0 |
| PEG-7 Glyceryl Cocoate | 2.5 |
| Dimethicone | 2.5 |
| Prunus Armeniaca (Apricot) Kernel Oil | 2.5 |
| Panthenol | 0.3 |
| Cocodimonium Hydroxypropyl Hydrolyzed Keratin | 0.7 |
| Hydrolyzed Keratin | 0.7 |
| Sodium Chloride | 0.8 |
| Cocamide MEA | 0.5 |
| Glycol Distearate | 0.2 |
| Sodium Benzoate | 0.7 |
| Citric Acid | ad pH 5.5 |
| Polyquaternium-10 | 0.5 |
| Laureth-4 | 0.4 |
| PEG-40 Hydrogenated Castor Oil | 0.2 |
| Hydrogenated Castor Oil | 0.7 |
| Laureth-7 | 0.5 |
| Hexyl Cinnamal | 0.1 |
| Propylene Glycol | 0.8 |
| Hexyl Salicylate | 0.3 |
| Glycerin | 0.5 |
| Linalool | 0.2 |
| Limonene | 0.1 |
| Dyes | q.s. |

Recipes 104: Shampoo

| Ingredient | % |
|---|---|
| Aqua | ad 100 |
| Sodium Coco Sulfate | 6.0 |
| Lauryl Glucoside | 4.0 |
| Glycerin | 3.5 |
| Sodium Lactate | 2.5 |
| SFX | 2.0 |
| Stearamidopropyl Dimethylamine | 1.0 |
| Betaine | 0.8 |
| Sodium Chloride | 0.9 |
| Simmondsia Chinensis Seed Oil | 0.8 |
| Sodium Cocoyl Glutamate | 0.7 |
| Disodium Cocoyl Glutamate | 0.5 |
| Xanthan Gum | 0.5 |
| Hydrated Silica | 0.5 |
| Parfum, Dyes | q.s. |

Recipes 105: Shampoo for Children

| Ingredient | % |
|---|---|
| Aqua | ad 100 |
| Sodium Coco Sulfate | 7.0 |
| Decyl Glucoside | 5.0 |
| Lactis Proteinum | 3.5 |
| Sorbitan Caprylate | 3.0 |
| SFX | 3.0 |
| Glycerin | 2.5 |
| Sodium Lactate | 2.5 |
| Alcohol | 2.0 |
| Hydrolyzed Wheat Protein | 0.7 |
| Hydrolyzed Wheat Starch | 0.7 |
| Sodium Chloride | 0.9 |
| Limonene | 0.1 |
| Citral | 0.1 |
| Phenethyl Alcohol | 0.1 |
| Dyes | q.s. |

Recipes 106: Intensive Cream Shampoo

| Ingredient | % |
|---|---|
| Aqua | ad 100 |
| Sodium Laureth Sulfate | 8.5 |
| Cocamidopropyl Betaine | 3.0 |
| SFX | 2.5 |
| Sodium Chloride | 2.5 |
| Panthenol | 0.5 |
| Hydrolyzed Keratin | 0.8 |
| Argania Spinosa Kernel Oil | 0.5 |
| Cocamide MEA | 0.5 |
| Glycol Distearate | 0.5 |
| PEG-7 Glyceryl Cocoate | 0.4 |
| Sodium Benzoate | 0.7 |
| Citric Acid | ad pH 5.0 |
| Laureth-4 | 0.3 |
| PEG-40 Hydrogenated Castor Oil | 0.4 |
| Hydrogenated Castor Oil | 0.4 |
| Styrene/Acrylates Copolymer | 0.5 |
| Dimethicone | 0.8 |
| Polyquaternium-7 | 0.9 |
| PEG-12 Dimethicone | 0.5 |
| Guar Hydroxypropyltrimonium Chloride | 0.3 |
| Benzyl Salicylate | 0.1 |
| Propylene Glycol | 0.8 |
| Glycerin | 0.5 |
| Limonene | 0.1 |
| Linalool | 0.1 |
| PEG-14m | 0.1 |
| Dyes | q.s. |

Recipes 107: Shampoo

| Ingredient | % |
|---|---|
| Aqua | ad 100 |
| Sodium Laureth Sulfate | 7.5 |
| Cocamidopropyl Betaine | 3.0 |
| SFX | 2.5 |
| Sodium Chloride | 2.0 |
| Sodium Xylenesulfonate | 1.7 |
| Cocamide MEA | 1.5 |
| Sodium Citrate | 1.5 |
| Citric Acid | ad pH 5.0 |
| Dimethiconol | 1.0 |
| Cassia Hydroxypropyltrimonium Chloride | 0.3 |
| Sodium Benzoate | 0.9 |
| Disodium EDTA | 0.7 |
| Panthenol | 0.7 |
| Panthenyl Ethyl Ether | 0.3 |
| Persea Gratissima (Avocado) Oil | 0.2 |
| Methylchloroisothiazolinone | 0.1 |
| Methylisothiazolinone | 0.1 |
| Parfum, Dyes | q.s. |

Recipes 108: Caffeine Shampoo

| Ingredient | % |
|---|---|
| Aqua | ad 100 |
| Sodium Laureth Sulfate | 6.0 |
| Laureth-2 | 4.0 |
| Disodium Laureth Sulfosuccinate | 2.5 |
| Sodium Lauroyl Glutamate | 2.5 |
| SFX | 2.0 |
| Sodium Chloride | 2.0 |
| Caffeine | 1.5 |
| Panthenol | 0.2 |
| PEG-120 Methyl Glucose Dioleate | 0.5 |
| Hydrolysed Wheat Protein | 0.7 |
| Citric Acid | ad pH 5.5 |
| Sodium Citrate | 0.5 |
| Menthol | 0.7 |
| PEG-40 Hydrogenated Castor Oil | 0.4 |
| Potassium Sorbate | 0.3 |
| Polyquaternium-7 | 0.4 |
| Disodium EDTA | 0.5 |
| Sodium Benzoate | 0.7 |
| Zinc PCA | 0.3 |
| Niacinamide | 0.1 |
| Limonene | 0.1 |
| Tocopherol | 0.1 |
| Phenoxyethanol | 0.5 |
| Methylparaben | 0.3 |
| Propylparaben | 0.3 |
| Dyes | q.s. |

Recipes 109: Power Grey Shampoo

| Ingredient | % |
|---|---|
| Aqua | ad 100 |
| Sodium Laureth Sulfate | 6.5 |
| Sodium Cocoamphoacetate | 4.5 |
| PEG-3 Distearate | 3.0 |
| Cocamidopropyl Betaine | 3.0 |
| SFX | 2.5 |
| Sodium Chloride | 2.5 |
| Caffeine | 1.5 |
| Panthenol | 0.2 |
| Phenoxyethanol | 0.7 |
| Coco Glucoside | 0.9 |
| Glyceryl Oleate | 0.4 |
| Phosphoric Acid | 0.2 |
| PEG-120 Methyl Glucose Dioleate | 0.9 |
| Menthol | 0.9 |
| Polyquaternium-7 | 0.7 |
| Sodium Hydroxide | 0.3 |
| Citric Acid | ad pH 5.5 |
| Niacinamide | 0.1 |
| Zinc PCA | 0.5 |
| Limonene | 0.1 |
| Sodium Benzoate | 0.2 |
| Methylparaben | 0.3 |
| Propylparaben | 0.3 |
| Dyes | q.s. |

Recipes 110: Shampoo

| Ingredient | % |
|---|---|
| Aqua | ad 100 |
| Lauryl Glucoside | 6.5 |
| Cocamidopropyl Betaine | 3.0 |
| Coco Glucoside | 2.5 |
| SFX | 2.0 |
| Glyceryl Oleate | 1.5 |
| Sodium Cocoyl Glutamate | 1.0 |
| Panthenol | 0.5 |
| Polyquaternium-10 | 0.3 |
| Chamomilla Recutita Extract | 0.5 |
| Glyceryl Caprylate | 0.3 |
| p-Anisic Acid | 0.05 |
| Citric Acid | ad pH 5.0 |
| Parfum, Dyes | q.s. |

Recipes 111: Caffeine Shampoo

| Ingredient | % |
|---|---|
| Aqua | ad 100 |
| Sodium Coco Sulfate | 6.0 |
| Glycerin | 5.5 |
| Lauryl Glucoside | 4.5 |
| Cocamidopropyl Betaine | 3.0 |
| SFX | 2.5 |
| Lauroyl Sarcosine | 2.0 |
| Caffeine | 1.0 |
| Xanthan Gum | 1.0 |
| Hydroxypropyl Guar Hydroxypropyltrimonium Chloride | 0.3 |
| Alcohol | 0.8 |
| Coffea Arabica Bean Extract | 0.3 |
| Panthenyl Ethyl Ether | 0.4 |
| Panthenol | 0.2 |
| Limonene | 0.1 |
| Linalool | 0.1 |
| Dyes | q.s. |

Recipes 112: Shampoo

| Ingredient | % |
|---|---|
| Aqua | ad 100 |
| Sodium Laureth Sulfate | 6.5 |
| Cocamidopropyl Betaine | 3.0 |
| Sodium Chloride | 3.0 |
| SFX | 2.0 |
| Panthenol | 0.2 |
| Benzophenone-4 | 0.9 |
| Glycol Distearate | 0.7 |
| Laureth-4 | 0.8 |
| Glycerin | 0.9 |
| Formic Acid | 0.2 |
| Decyl Glucoside | 0.9 |
| Polyquaternium-10 | 0.4 |
| Niacinamide | 0.1 |
| Sodium Benzoate | 0.1 |
| Potassium Sorbate | 0.2 |
| Citric Acid | ad pH 5.0 |
| Parfum, Dyes | q.s. |

Recipes 113: Shampoo

| Ingredient | % |
|---|---|
| Aqua | ad 100 |
| Sodium Laureth Sulfate | 8.5 |
| Cocamidopropyl Betaine | 3.0 |
| Sodium Chloride | 2.0 |
| Disodium Cocoamphodiacetate | 2.0 |
| SFX | 2.0 |
| Sodium Benzoate | 0.7 |
| Panthenol | 0.2 |
| Sodium Laureth-8 Sulfate | 0.7 |
| Citric Acid | ad pH 5.0 |
| Sodium Cocoyl Glutamate | 0.8 |
| Glycol Distearate | 0.9 |
| Styrene/Acrylates Copolymer | 0.5 |
| Sodium Oleth Sulfate | 0.7 |
| Coco Glucoside | 0.9 |
| Cocamide MEA | 0.7 |
| Isopropyl Alcohol | 0.2 |
| Parfum, Dyes | q.s. |

Recipes 114: Volume Shampoo

| Ingredient | % |
|---|---|
| Aqua | ad 100 |
| Sodium Laureth Sulfate | 7.0 |
| Cocamidopropyl Betaine | 3.0 |
| Coco Glucoside | 3.0 |
| SFX | 2.0 |
| Nymphaea Coerulea Flower Extract | 2.0 |
| Hydrolyzed Rice Protein | 2.0 |
| Cocamide MEA | 2.0 |
| Polyquaternium-10 | 0.5 |
| Malic Acid | ad pH 5.5 |
| Disodium EDTA | 0.5 |
| Propylene Glycol | 0.5 |
| Benzophenone-4 | 0.3 |
| Ethylhexyl Methoxycinnamate | 0.3 |
| Sodium Chloride | 0.9 |
| Benzyl Alcohol | 0.9 |
| PPG-9 | 0.2 |
| Methylisothiazolinone | 0.1 |
| Methylparaben | 0.1 |
| Propylparaben | 0.1 |
| Parfum, Dyes | q.s. |

Recipes 115: Shampoo

| Ingredient | % |
|---|---|
| Aqua | ad 100 |
| Sodium Laureth Sulfate | 6.5 |
| Coco Glucoside | 5.0 |
| Cocamidopropyl Betaine | 3.0 |
| SFX | 3.0 |
| Glycol Distearate | 2.8 |
| Dimethicone | 1.7 |
| Laureth-23 | 1.5 |
| Laureth-4 | 1.5 |
| Polyquaternium-7 | 0.5 |
| Cocamide MEA | 0.7 |
| Cetyl Alcohol | 0.4 |
| Simmondsia Chinensis Seed Oil | 0.8 |
| Panthenol | 0.3 |
| Malic Acid | ad pH 5.5 |
| Glycine | 0.7 |
| Benzyl Alcohol | 0.7 |
| Disodium EDTA | 0.5 |
| Sodium Hydroxide | 0.2 |
| Guar Hydroxypropyltrimonium Chloride | 0.5 |
| Ethylhexyl Methoxycinnamate | 0.7 |
| Methylparaben | 0.3 |
| Propylparaben | 0.3 |
| PPG-9 | 0.2 |
| Benzophenone-4 | 0.3 |
| Sodium Chloride | 0.8 |
| DMDM Hydantoin | 0.1 |
| Hexyl Cinnamal | 0.1 |
| Limonene | 0.1 |
| Dyes | q.s. |

Recipes 116: Shampoo

| Ingredient | % |
|---|---|
| Aqua | ad 100 |
| Sodium Laureth Sulfate | 5.0 |
| Sodium Lauryl Sulfate | 5.0 |
| Cocamidopropyl Betaine | 3.0 |
| SFX | 2.5 |
| Olea Europaea Fruit Oil | 2.0 |
| Cocamide MEA | 2.0 |
| Sodium Chloride | 1.9 |
| Glycol Distearate | 1.7 |
| Benzyl Alcohol | 1.0 |
| Polyquaternium-10 | 0.5 |
| Hydroxypropyl Methylcellulose | 0.5 |
| Silicone Quaternium-18 | 0.9 |
| Malic Acid | ad pH 5.5 |
| Disodium EDTA | 0.5 |
| Sodium Xylenesulfonate | 0.7 |
| Trideceth-6 | 0.7 |
| Trideceth-12 | 0.5 |
| DMDM Hydantoin | 0.2 |
| Methylparaben | 0.3 |
| Propylparaben | 0.3 |
| Linalool | 0.1 |
| Hexyl Cinnamal | 0.1 |
| Butylphenyl Methylpropional | 0.1 |
| Limonene | 0.1 |
| Dyes | q.s. |

Recipes 117: Shampoo

| Ingredient | % |
|---|---|
| Aqua | ad 100 |
| Sodium Laureth Sulfate | 3.0 |
| Sodium Lauryl Sulfate | 3.5 |
| Glycol Distearate | 3.3 |
| Dimethicone | 3.0 |
| Cocamidopropyl Betaine | 2.5 |
| SFX | 2.0 |
| Benzyl Alcohol | 0.7 |
| Betaine | 0.4 |
| Cetyl Alcohol | 0.7 |
| Cocamide MEA | 0.7 |
| Glyceryl Oleate | 0.5 |
| Propylene Glycol | 0.6 |
| Menthol | 0.7 |
| Guar Hydroxypropyltrimonium Chloride | 0.4 |
| Butylene Glycol | 0.5 |
| Menthyl Lactate | 0.2 |
| Disodium EDTA | 0.5 |
| Tocopheryl Acetate | 0.1 |
| Laureth-4 | 0.7 |
| Niacinamide | 0.1 |
| Maltodextrin | 0.2 |
| Sodium Ascorbyl Phosphate | 0.1 |
| PPG-9 | 0.2 |
| Salicylic Acid | 0.7 |
| Pyridoxine HCl | 0.1 |
| Malic Acid | ad pH 5.0 |
| Laureth-23 | 0.5 |
| Silica | 0.7 |
| Sodium Chloride | 0.7 |
| Sodium Hydroxide | 0.3 |
| Methylchloroisothiazolinone | 0.1 |
| Methylisothiazolinone | 0.1 |
| Parfum, Dyes | q.s. |

Recipes 118: Shampoo

| Ingredient | % |
|---|---|
| Aqua | ad 100 |
| Cocamidopropyl Betaine | 7.0 |
| Sodium Methyl Cocoyl Taurate | 4.0 |
| SFX | 1.0 |
| Sodium Chloride | 2.0 |
| Sodium C14-16 Olefin Sulfonate | 1.0 |
| PEG-120 Methyl Glucose Dioleate | 1.0 |
| Glycol Distearate | 1.0 |
| Sodium Benzoate | 1.0 |
| Caprylyl/Capryl Glucoside | 0.7 |
| PEG-7 Glyceryl Cocoate | 0.7 |
| Citric Acid | ad pH 5.3 |
| Parfum | 0.2 |
| Polyquaternium-10 | 0.2 |
| Coconut Acid | 0.1 |
| Laureth-4 | 0.1 |
| Hydrogenated Castor Oil | 0.1 |
| Glycerin | 0.1 |
| Lactic Acid | 0.1 |
| Propylene Glycol | 0.1 |
| Hydrolyzed Keratin | 0.1 |
| Ceramide NP | 0.05 |
| Panthenol | 0.05 |

Recipes 119: Conditioning Shampoo

| Ingredient | % |
|---|---|
| Aqua | ad 100 |
| Sodium Cocoamphoacetate | 5.0 |
| Cocamidopropyl Betaine | 4.0 |
| SFX | 1.5 |
| Starch Hydroxypropltrimonium chloride | 1.2 |
| Isostearamide MIPA; Glyceryl Laurate | 1.2 |
| Isoamyl Laurate | 1.0 |
| Bis-(Isostearoyl/Oleoyl Isopropyl) Dimonium Methosulfate | 0.5 |
| Citric Acid | 3.0 |
| Perfume, Preservative, Dyes | q.s. |

Recipes 120: Conditioning Shampoo

| Ingredient | % |
|---|---|
| Aqua | ad 100 |
| Sodium Laureth Sulfate | 7.0 |
| Cocamidopropyl Betaine | 1.5 |
| SFXs | 1.5 |
| Palmitamidopropyltrimonium Chloride | 1.0 |
| Polysilicone-19 | 1.0 |
| PEG-18 Glyceryl Oleate/Cocoate | 2.5 |
| Citric Acid | ad pH 5.5 |
| Perfume, Preservative | q.s. |

Recipes 121: Conditioning Shampoo

| Ingredient | % |
|---|---|
| Aqua | ad 100 |
| Sodium Cocoamphoacetate | 5.0 |
| Cocamidopropyl Betaine | 3.0 |
| Disodium Lauryl Sulfosuccinate | 3.0 |
| SFXs | 1.5 |
| Palmitamidopropyltrimonium Chloride | 2.0 |
| Isostearamide MIPA, Glyceryl Laurate | 1.0 |
| PEG-200 Hydrogenated Glyceryl Palmate; | 1.0 |
| PEG-7 Glyceryl Cocoate | |
| Methoxy PEG/PPG-7/3 Aminopropyl Dimethicone | 0.5 |
| Starch Hydroxypropltrimonium chloride | 0.3 |
| Polyquaternium-10 | 0.2 |
| Citric Acid | ad pH 5.5 |
| Perfume, Preservative | q.s. |

Recipes 122: Conditioning Shampoo

| Ingredient | % |
|---|---|
| Aqua | ad 100 |
| Sodium Laureth Sulfate | 6.0 |
| Cocamidopropyl Betaine | 3.0 |
| SFX | 3.0 |
| Glycol Distearate, Laureth-4, Cocamidopropyl Betaine | 2.0 |
| PEG-120 Methyl Glucose Dioleate | 1.7 |
| SFX | 1.5 |
| Sorbitan Sesquicaprylate | 1.0 |
| Isoamyl Laurate | 1.0 |
| Sodium Chloride | 1.0 |
| Silicone-Quaternium-22 | 0.8 |
| Hydroxypropyl Guar Hydroxypropyltrimonium Chloride | 0.5 |
| Creatine | 0.5 |
| Citric Acid | ad pH 5.5 |
| Perfume, Preservative | q.s. |

Recipes 123: Natural Shampoo

| Ingredient | % |
|---|---|
| Aqua | ad 100 |
| Sodium Coco-Sulfate | 9.0 |
| Glycerin | 5.0 |
| Lauryl Glucoside | 2.5 |
| SFX | 2.5 |
| Sodium Lactate | 1.0 |
| Betaine | 1.0 |
| Hamamelis Virginiana Leaf Extract | 1.0 |
| Alcohol Denat. | 1.0 |
| Sodium/Disodium Cocoyl Glutamate | 0.8 |
| Maris Sal | 0.5 |
| Dipotassium Glycyrrhizate | 0.5 |
| Glycerophosphocholine | 0.1 |
| Parfum, Preservative | q.s. |

Recipes 124: Natural Shampoo

| Ingredient | % |
|---|---|
| Aqua | ad 100 |
| Sodium Coco-sulfate | 6.0 |
| Glycerin | 5.0 |
| Coco-glucoside | 4.0 |
| Lauryl Glucoside | 3.0 |
| SFX | 2.0 |
| Glyceryl Oleate | 1.5 |
| Sodium Chloride | 1.0 |
| Calendula Officinalis Flower Extract | 0.5 |
| Parfum | 0.5 |
| Starch Hydroxypropyltrimonium chloride | 0.3 |
| Glyceryl Undecylenate | 0.3sarch |
| Polyglyceryl-4 Caprate | 0.3 |
| Xanthan Gum | 0.3 |
| Locust bean Gum | |
| Lysolecithin | 0.2 |
| Coco-Caprylate | 0.1 |
| Magnolia Officinalis Bark Extract | 0.1 |
| Tocopherol | 0.1 |
| Squalene | 0.1 |

Recipes 125: Natural Shampoo

| Ingredient | % |
|---|---|
| Aqua | ad 100 |
| Sodium Lauroyl Methyl Isethionate | 5.0 |
| Disodium Cocoamphodipropionate | 4.0 |
| Acrylates Copolymer | 2.0 |
| Hydroxyethylcellulose | 2.0 |
| SFX | 2.0 |
| Cocos Nucifera Oil | 0.8 |
| Panthenol | 0.4 |
| Tocopherol | 0.4 |
| Tocopheryl Acetate | 0.2 |
| Cetyl Triethylmonium Dimethicone PEG-8 Succinate | 0.2 |
| Glycol Distearate | 0.2 |
| Laureth-4 | 0.2 |
| Cocamidopropyl Betaine | 0.2 |
| Ethylhexyl Salicylate | 0.1 |
| Disodium EDTA | 0.1 |
| Ethylhexylglycerin | 0.1 |
| Glycerin | 0.1 |
| Hexylene Glycol | 0.1 |
| Sodium Coco PG-dimonium Chloride Phosphate | 0.1 |
| BHT | 0.1 |
| Citric Acid | 0.1 |
| Phenoxyethanol | 0.1 |
| Formic Acid | 0.1 |
| Parfum | q.s. |

Recipes 126: Natural Shampoo

| Ingredient | % |
|---|---|
| Aqua | ad 100 |
| Moroccan Lava Clay | 5.0 |
| Behenyl Alcohol | 3.0 |
| Glycerin | 2.5 |
| Glyceryl Stearate Citrate | 2.0 |
| Lauryl Glucoside | 2.0 |
| Coco-glucoside | 2.0 |
| Helianthus Annuus Hybrid Oil | 1.5 |
| Prunus Armeniaca Kernel Oil | 1.5 |
| SFX | 1.0 |
| Argania Spinosa Kernel Oil | 1.0 |
| Magnolia Officinalis Bark Extract | 1.0 |
| Triethyl Citrate, Glyceryl Caprylate Benzoic Acid | 1.0 |
| Xanthan Gum | 0.8 |
| Sodium Lactate | 0.5 |
| Benzyl Alcohol | 0.3 |
| Lactic Acid | 0.2 |
| Parfum | q.s. |

Recipes 127: Natural Shampoo

| Ingredient | % |
|---|---|
| Aqua | ad 100 |
| Cocamidopropyl Betaine | 7.0 |
| Cetrimonium Chloride | 5.0 |
| Decyl Glucoside | 5.0 |
| Sodium Chloride | 2.0 |
| SFX | 1.0 |
| Sorbitan Caprylate | 0.8 |
| Cocos Nucifera Oil | 0.8 |
| Inulin | 0.5 |
| Benzyl Alcohol | 0.2 |
| Cetearyl Alcohol | 0.2 |
| Xanthan Gum | 0.2 |
| Sodium Cocoyl Glutamate | 0.1 |
| Parfum | 0.1 |
| Glycerin | 0.2 |
| Argania Spinosa Kernel Oil | 0.1 |
| Titanium Dioxide | 0.1 |
| Hydroxypropyl Guar Hydroxypropyltrimonium Chloride | 0.1 |
| Lactic Acid | 0.1 |
| Hexyl Cinnamal | 0.1 |
| Sodium Hydroxide | 0.1 |
| Ascorbyl Palmitate | 0.1 |
| Tocopherol | 0.1 |

Recipes 128: Natural Shampoo

| Ingredient | % |
|---|---|
| Aqua | ad 100 |
| Glycerin | 5.0 |
| Sodium Coco-sulfate | 5.0 |
| Lauryl Glucoside | 4.0 |
| SFX | 4.0 |
| Betaine | 3.0 |
| Maris Sal | 2.0 |
| Camellia Oleifera Leaf Extract | 1.5 |
| Pisum Sativum Peptide | 1.5 |
| Oryza Sativa Extract | 1.0 |
| Pisum Sativum Seed Extract | 0.5 |
| Disodium Cocoyl Glutamate | 0.4 |
| Sodium Cocoyl Glutamate | 0.4 |
| PCA Glyceryl Oleate | 0.2 |
| Alcohols | 0.1 |
| Parfum | q.s. |

Recipes 129: Anti-Hair Loss Tonic

| Ingredient | % |
|---|---|
| Aqua | ad 100 |
| Alcohol Denat. | 50.0 |
| PEG-40 Hydrogenated Castor Oil | 2.5 |
| SFX | 1.5 |
| PEG-7 Glyceryl Cocoate | 2.0 |
| Sphinganine | 0.1 |
| Parfum, Preservative | q.s. |

Recipes 130: Shower Oil

| Ingredient | % |
|---|---|
| Aqua | ad 100 |
| Magnesium Laureth Sulfate | 6.0 |
| PEG-7 Glyceryl Cocoate | 5.0 |
| Hydrogenated Starch Hydrolysate | 3.0 |
| Cocamidopropyl Betaine | 3.0 |
| SFX | 3.0 |
| Decyl Glucoside | 1.0 |
| Prunus Amygdalus Dulcis (Sweet Almond) Oil | 1.0 |
| Olive Oil | 0.5 |
| PEG-7 Esters | 0.2 |
| Parfum | 0.1 |
| Polysorbate 20 | 0.1 |
| Sodium Benzoate | 0.2 |
| Citric Acid | 0.1 |
| PEG-6 Caprylic/Capric Glycerides | 0.1 |
| PEG-120 Methyl Glucose Dioleate | 0.1 |
| Disodium EDTA | 0.1 |

Recipes 131: Shower Mousse

| Ingredient | % |
|---|---|
| Aqua | ad 100 |
| Sodium Lauroyl Glutamate | 15.0 |
| Isobutane | 6.0 |
| Cocamidopropyl Betaine | 4.0 |
| Glycerin | 3.0 |
| SFX | 3.0 |
| Sodium Chloride | 2.6 |
| Sodium Lauroyl Glycinate | 1.5 |
| Propane | 1.0 |
| Butane | 0.6 |
| Citric Acid | 0.4 |
| Iodopropynyl Butylcarbamate | 0.3 |
| Lauric Acid | 0.3 |
| Parfum | 0.1 |
| Polysorbate 20 | 0.1 |
| Sodium Benzoate | 0.1 |
| Sodium Hydroxide | 0.1 |
| Stearic Acid | 0.1 |
| Tetrasodium EDTA | 0.1 |

Recipes 132: Shower Jelly

| Ingredient | % |
|---|---|
| Glycerin | 3.5 |
| Sodium Laureth Sulfate | 3.5 |
| SFX | 3.0 |
| Propylene Glycol | 2.0 |
| Chondrus Crispus Extract | 2.0 |
| Pyrus Malus Juice | 1.0 |
| Vitis Vinifera Juice | 0.5 |
| Parfum | 0.2 |
| Butylphenyl Methylpropional | 0.1 |

Recipes 133: Shower Jelly

| Ingredient | % |
|---|---|
| Aqua | ad 100 |
| Glycerin | 9.0 |
| Coco-Glucoside | 5.0 |
| Decyl Glucoside | 5.0 |
| SFX | 2.0 |
| Chondrus crispus extract | 1.0 |
| Parfum | 0.2 |
| Citric Acid | 0.2 |
| Sodium Anisate | 0.1 |
| Sodium Levulinate | 0.1 |

Recipes 134: Shower Scrub

| Ingredient | % |
|---|---|
| Aqua | ad 100 |
| Maris Sal | 15.0 |
| Sodium Laureth Sulfate | 9.0 |
| SFX | 2.0 |
| Sodium Cocoamphoacetate | 1.0 |
| Betaine | 1.0 |
| Citrus Limon (Lemon) Peel Powder | 0.5 |
| Lactic Acid | ad pH 5.5 |
| Parfum, Dyes | q s. |

Recipes 135: Cellulose Scrub

| Ingredient | % |
|---|---|
| Aqua | ad 100 |
| SFX | 7.0 |
| Coco Glucoside | 6.0 |
| Glycerin | 5.0 |
| Glyceryl Stearate | 5.0 |
| Citrus Aurantium Dulcis Fruit Water | 4.5 |
| Helianthus Annuus Hybrid Oil | 4.0 |
| Cellulose | 2.0 |
| Jojoba Esters | 2.0 |
| Cetyl Alcohol | 2.0 |
| Simmondsia Chinensis (Jojoba) Seed Oil | 2.0 |
| Olea Europaea (Olive) Fruit Oil | 2.0 |
| Hydrogenated Castor Oil | 0.8 |
| Prunus Armeniaca (Apricot) Kernel Oil | 0.5 |
| Leontopodium Alpinum Extract | 0.1 |
| Xanthan Gum | 0.3 |
| Benzyl Alcohol | 0.2 |
| Parfum | 0.1 |
| Dehydroacetic Acid | 0.1 |
| Citric acid | ad pH 5.0 |
| Lactic acid | 0.1 |
| Sodium Hydroxide | 0.1 |
| Parfum, Dyes | q. s. |

Recipes 136: Exfoliating Body Scrub

| Ingredient | % |
|---|---|
| Aqua | ad 100 |
| Coco-Glucoside | 7.0 |
| Alcohol | 6.0 |
| Glycerin | 5.0 |
| Glycine Soja Oil | 5.0 |
| Hydrated Silica | 4.0 |
| Xanthan Gum | 3.0 |
| SFX | 3.0 |
| Disodium/Sodium Cocoyl Glutamate | 1.0 |
| Coffea Arabica (Coffee) Seed Powder | 1.0 |
| Cocos Nucifera (Coconut) Shell Powder | 1.0 |
| Citric Acid | ad pH 5.2 |
| Glyceryl Oleate | 0.5 |
| Parfum | 0.1 |
| Preservative, Dyes | q.s. |

Recipes 137: Dry Shampoo

| Ingredient | % |
|---|---|
| Butane, Propane | 53.0 |
| Alcohol Denat. | 36.0 |
| Aluminum Starch Octenylsuccinate | 3.0 |
| Panthenol | 1.5 |
| Tocopherol | 1.0 |
| Cocos Nucifera Oil | 1.0 |
| Curcuma Longa (Turmeric) Root Extract | 0.6 |
| Silica | 0.5 |
| Sodium Bicarbonate | 0.5 |
| Charcoal Powder | 0.4 |
| Hydrolyzed Rice Protein | 0.4 |
| Aminopropyl Phenyl Trimethicone | 0.4 |
| Benzophenone-4 | 0.3 |
| Polysorbate 20 | 0.3 |
| SFX | 0.3 |
| Cyclopentasiloxane | 0.2 |
| Cyclohexasiloxane | 0.2 |
| Glycerin | 0.2 |
| Aqua | 0.2 |
| Parfum | q.s. |

Recipes 138: Dry Shampoo

| | % |
|---|---|
| Butane, Isobutane, Propane | 55.0 |
| Oryza Sativa (Rice) Starch | 25.0 |
| Alcohol denat. | 18.0 |
| Parfum | 1.0 |
| SFX | 0.5 |
| Distearyldimonium Chloride | 0.3 |
| Cetrimonium Chloride | 0.2 |

Recipes 139: Shower Gel

| Ingredient | % |
|---|---|
| Aqua | ad 100 |
| Sodium Laureth Sulfate | 8.5 |
| Cocamidopropyl Betaine | 3.0 |
| Sodium Chloride | 2.7 |
| SFX | 2.5 |
| Guar Gum | 0.5 |
| Polyquaternium-7 | 0.5 |
| Bisabolol | 0.2 |
| Trideceth-9 | 0.5 |
| Sodium Sulfate | 0.9 |
| Tetrasodium EDTA | 0.2 |
| Citric Acid | ad pH 5.5 |
| Sodium Benzoate | 0.7 |
| Benzyl Alcohol | 0.7 |
| Benzyl Salicylate | 0.1 |
| Geraniol | 0.2 |
| Hexyl Cinnamal | 0.1 |
| Limonene | 0.1 |
| Linalool | 0.1 |
| Dyes | q.s. |

Recipes 140: Body Wash

| Ingredient | % |
|---|---|
| Water | ad 100 |
| Sodium Laureth Sulfate | 4.0 |
| Sodium Lauryl Sulfate | 4.0 |
| Sodium Chloride | 3.3 |
| Cocamidopropyl Betaine | 3.0 |
| SFX | 3.0 |
| Sodium Benzoate | 1.5 |
| Citric Acid | ad pH 5.5 |
| Disodium EDTA | 0.5 |
| Zea Mays (Corn) Silk Extract | 0.5 |
| Methylchloroisothiazolinone | 0.1 |
| Methylisothiazolinone | 0.1 |
| Potassium Sorbate | 0.2 |
| Parfum, Dyes | q.s. |

Recipes 141: Cool Down Shower Gel

| Ingredient | % |
|---|---|
| Aqua | ad 100 |
| Sodium Laureth Sulfate | 8.0 |
| Cocamidopropyl Betaine | 3.0 |
| SFX | 3.0 |
| Glycerin | 2.7 |
| Acrylates Copolymer | 2.5 |
| Sodium Chloride | 2.2 |
| Phenoxyethanol | 1.5 |
| Taurine | 1.5 |
| Benzyl Alcohol | 1.0 |
| Sodium Hydroxide | 0.5 |
| PEG-40 Hydrogenated Castor Oil | 0.7 |
| Citric Acid | ad pH 5.0 |
| Mannitol | 0.7 |
| Benzophenone-4 | 0.3 |
| Ethylhexyl Salicylate | 0.5 |
| Cellulose | 0.5 |
| Linalool | 0.1 |
| Butyl Methoxydibenzoylmethane | 0.2 |
| Hydroxypropyl Methylcellulose | 0.2 |
| Dyes | q.s. |

Recipes 142: Shower Gel

| Ingredient | % |
|---|---|
| Aqua | ad 100 |
| Cocamidopropyl Betaine | 5.0 |
| Sodium Myreth Sulfate | 4.5 |
| Sodium Laureth Sulfate | 4.0 |
| SFX | 3.5 |
| PEG-7 Glyceryl Cocoate | 3.2 |
| Glycerin | 3.0 |
| Glyceryl Glucoside | 2.5 |
| Bisabolol | 2.0 |
| Chamomilla Recutita (Matricaria) Flower Extract | 2.2 |
| Sodium Chloride | 2.2 |
| PEG-40 Hydrogenated Castor Oil | 2.0 |
| Citric Acid | ad pH 5.0 |
| PEG-90 Glyceryl Isostearate | 1.2 |
| PEG-200 Hydrogenated Glyceryl Palmate | 1.2 |
| Benzophenone-4 | 1.0 |
| Laureth-2 | 0.7 |
| Polyquaternium-7 | 0.5 |
| BHT | 0.2 |
| Sodium Benzoate | 0.2 |
| Methylparaben | 0.2 |
| Benzyl Alcohol | 0.2 |
| Citronellol | 0.1 |
| Limonene | 0.1 |
| Dyes | q.s. |

Recipes 143: Shower Gel

| Ingredient | % |
|---|---|
| Aqua | ad 100 |
| Sodium Laureth Sulfate | 8.0 |
| Cocamidopropyl Betaine | 3.0 |
| Acrylates Copolymer | 3.0 |
| PEG-7 Glyceryl Cocoate | 2.7 |
| SFX | 2.5 |
| Helianthus Annuus Seed Oil | 1.5 |
| Glycerin | 1.5 |
| Sodium Chloride | 1.0 |
| PEG-40 Hydrogenated Castor Oil | 1.0 |
| Disodium Cocoyl Glutamate | 1.0 |
| Trisodium EDTA | 1.0 |
| Propylene Glycol | 1.0 |
| Microcrystalline Cellulose | 0.9 |
| Benzophenone-4 | 0.5 |
| Sodium Sulfate | 0.7 |
| Phenoxyethanol | 0.7 |
| Acacia Gum | |
| Propylparaben | 0.1 |
| Methylparaben | 0.1 |
| Benzyl Alcohol | 0.2 |
| Parfum, Dyes | q.s. |

Recipes 144: Body Wash

| Ingredient | % |
|---|---|
| Aqua | ad 100 |
| Sodium Hydroxypropyl Starch Phosphate | 6.0 |
| Sodium Laureth Sulfate | 5.5 |
| Cocamidopropyl Betaine | 3.0 |
| SFX | 3.0 |
| Lauric Acid | 2.5 |
| Petrolatum | 2.0 |
| Sodium Cocoyl Glycinate | 2.0 |
| Glycerin | 1.7 |
| Sodium Lauroyl Isethionate | 1.5 |
| Glycine Soja (Soybean) Oil | 1.0 |
| Helianthus Annuus (Sunflower) Oil | 1.0 |
| Sodium Chloride | 1.0 |
| Stearic Acid | 0.7 |
| Guar Hydroxypropyltrimonium Chloride | 0.3 |
| DMDM Hydantoin | 0.1 |
| Tallow Acid | 0.2 |
| Palmitic Acid | 0.1 |
| Sodium Isethionate | 0.2 |
| BHT | 0.1 |
| Tetrasodium EDTA | 0.2 |
| Methylisothiazolinone | 0.3 |
| Iodopropynyl Butylcarbamate | 0.1 |
| Titanium Dioxide | 0.1 |
| Parfum, Dyes | q.s. |

Recipes 145: Anti-Aging Body Wash

| Ingredient | % |
|---|---|
| Aqua | ad 100 |
| Petrolatum | 5.0 |
| Mineral (Paraffinum Liquidum) Oil | 4.0 |
| Sodium Trideceth Sulfate | 3.5 |
| Sodium Lauryl Sulfate | 3.0 |
| Sodium Lauroamphoacetate | 3.0 |
| Sodium Chloride | 2.3 |
| SFX | 2.0 |
| Trideceth-3 | 1.0 |
| Simmondsia Chinensis (Jojoba) Butter | 0.7 |
| Niacinamide | 0.1 |
| Panthenol | 0.3 |
| Soluble Collagen | 0.5 |
| Citric Acid | ad pH 5.5 |
| Disodium EDTA | 0.3 |
| Guar Hydroxypropyltrimonium Chloride | 0.3 |
| Methylchloroisothiazolinone | 0.1 |
| Methylisothiazolinone | 0.1 |
| PEG-90m | 0.7 |
| Sodium Benzoate | 0.3 |
| Sodium Hydroxide | 0.1 |
| Xanthan Gum | 0.5 |
| Calcium Alginate | .1 |
| Parfum, Dyes | q.s. |

Recipes 146: Shower Gel

| Ingredient | % |
|---|---|
| Aqua | ad 100 |
| Sodium Laureth Sulfate | 9.0 |
| Cocamidopropyl Betaine | 3.0 |
| SFX | 3.0 |
| Glycerin | 3.0 |
| Sodium Chloride | 2.8 |
| PEG-7 Glyceryl Cocoate | 2.5 |
| PEG-40 Hydrogenated Castor Oil | 2.5 |
| Citric Acid | ad pH 5.5 |
| Sodium Benzoate | 1.0 |
| Potassium Sorbate | 0.7 |
| Alcohol | 0.5 |
| Citronellol | 0.2 |
| Benzyl Alcohol | 0.7 |
| Limonene | 0.1 |
| Linalool | 0.2 |
| Geraniol | 0.2 |
| Hexyl Cinnamal | 0.1 |
| Ethylhexyl Salicylate | 0.1 |
| Octocrylene | 0.3 |
| Butyl Methoxydibenzoylmethane | 0.2 |
| Dyes | q.s. |

Recipes 147: Shower Gel

| Ingredient | % |
|---|---|
| Aqua | ad 100 |
| Alcohol | 6.0 |
| Coco Glucoside | 6.0 |
| Glycerin | 3.0 |
| SFX | 3.0 |
| Xanthan Gum | 1.5 |
| Disodium Cocoyl Glutamate | 2.0 |
| Citric Acid | ad pH 5.5 |
| Citrus Aurantifolia (Lime) Oil | 0.5 |
| Sodium Cocoyl Glutamate | 0.7 |
| Glyceryl Oleate | 0.3 |
| Sodium Lauroyl Lactylate | 0.8 |
| Limonene | 0.1 |
| Linalool | 0.1 |
| Dyes | q.s. |

Recipes 148: Shower Gel for Men

| Ingredient | % |
|---|---|
| Aqua | ad 100 |
| Sodium Laureth Sulfate | 9.5 |
| Cocamidopropyl Betaine | 3.0 |
| Cocamide MEA | 3.0 |
| SFX | 3.0 |
| Glycerin | 2.5 |
| PPG-12 | 2.0 |
| Sodium Chloride | 2.0 |
| Citric Acid | ad pH 5.5 |
| Sodium Benzoate | 1.0 |
| Phenoxyethanol | 0.7 |
| Benzyl Alcohol | 0.7 |
| Butylphenyl Methylpropional | 0.1 |
| Citronellol | 0.1 |
| Geraniol | 0.2 |
| Hexyl Cinnamal | 0.1 |
| Limonene | 0.1 |
| Linalool | 0.1 |
| Dyes | q.s. |

Recipes 149: Moisturising Shower Cream

| Ingredient | % |
|---|---|
| Aqua | ad 100 |
| Sodium Laureth Sulfate | 8.5 |
| Cocamidopropyl Betaine | 3.0 |
| Glycerin | 2.5 |
| SFX | 2.0 |
| Sodium Chloride | 1.7 |
| Cocamide MEA | 1.5 |
| Styrene/Acrylates Copolymer | 1.5 |
| Sodium Salicylate | 1.0 |
| Sodium Benzoate | 1.0 |
| Polyquaternium-7 | 1.0 |
| Citric Acid | ad pH 5.0 |
| Tetrasodium EDTA | 1.0 |
| Glycol Distearate | 0.9 |
| Laureth-4 | 0.5 |
| Magnolia Officinalis Flower Extract | 0.2 |
| Butyrospermum Parkii Butter Extract | 0.2 |
| Prunus Amygdalus Dulcis (Sweet Almond) Extract | 0.1 |
| Tapioca Starch polymethylsilsesquioxane | 0.5 |
| Benzyl Benzoate | 0.1 |
| Cinnamyl Alcohol | 0.1 |
| Hexyl Cinnamal | 0.1 |
| Parfum, Dyes | q.s. |

Recipes 150: Shower Cream

| Aqua | ad 100 |
|---|---|
| Ammonium Lauryl Sulfate | 6.0 |
| Coco Glucoside | 3.5 |
| Cocamidopropyl Betaine | 2.0 |
| Sodium Cocoamphoacetate | 2.0 |
| SFX | 1.5 |
| Argania Spinosa Kernel Oil | 1.0 |
| Butyrospermum Parkii Butter | 1.0 |
| Glycerin | 1.0 |
| Polyquaternium-7 | 1.0 |
| Styrene/Acrylates Copolymer | 1.0 |
| Gelatin Crosspolymer | 0.5 |
| Sodium Chloride | 0.5 |
| Citric Acid | ad pH 5.0 |
| Sodium Benzoate | 0.3 |
| Sodium Salicylate | 0.1 |
| Linalool | 0.1 |
| Dyes | q.s. |

Recipes 151: Shower Cream

| Ingredient | % |
|---|---|
| Aqua | ad 100 |
| Sodium Laureth Sulfate | 7.0 |
| Cocamidopropyl Betaine | 3.0 |
| SFX | 2.0 |
| Sodium Chloride | 1.9 |
| Polyquaternium-7 | 1.5 |
| Cocamide MEA | 1.5 |
| PEG-7 Glyceryl Cocoate | 1.0 |
| Laureth-10 | 1.0 |
| Glycol Distearate | 0.8 |
| Citric Acid | ad pH 5.0 |
| Linalool | 0.1 |
| Limonene | 0.1 |
| Benzyl Salicylate | 0.1 |
| Butylphenyl Methylpropional | 0.1 |
| alpha-Isomethyl lonone | 0.1 |
| Hexyl Cinnamal | 0.1 |
| Citronellol | 0.1 |
| Sodium Benzoate | 0.7 |
| Sodium Salicylate | 0.1 |
| Dyes | q.s. |

Recipes 152: Shower Cream

| Ingredient | % |
|---|---|
| Aqua | ad 100 |
| Glycerin | 7.5 |
| Glycine Soja Oil | 3.0 |
| Lauryl Glucoside | 3.0 |
| Sodium Coco Sulfate | 3.0 |
| SFX | 2.5 |
| Alcohol | 1.5 |
| Xanthan Gum | 1.5 |
| Butyrospermum Parkii Butter | 1.2 |
| Sodium Cetearyl Sulfate | 1.0 |
| Sodium Cocoyl Glutamate | 1.0 |
| Disodium Cocoyl Glutamate | 1.0 |
| Tocopherol | 0.1 |
| Helianthus Annuus Seed Oil | 0.3 |
| Limonene | 0.1 |
| Benzyl Salicylate | 0.1 |
| Linalool | 0.1 |
| Dyes | q.s. |

Recipes 153: Shower Gel

| Ingredient | % |
|---|---|
| Aqua | ad 100 |
| Sodium Coco Sulfate | 5.0 |
| Glycerin | 5.0 |
| Lauryl Glucoside | 4.0 |
| Sodium Lactate | 2.5 |
| SFX | 2.0 |
| Sodium Cocoyl Glutamate | 2.0 |
| Disodium Cocoyl Glutamate | 2.0 |
| Alcohol | 1.0 |
| Prunus Cerasus Fruit Extract | 1.0 |
| Limonene | 0.1 |
| Coumarin | 0.2 |
| Linalool | 0.1 |
| Citral | 0.1 |
| Dyes | q.s. |

Recipes 154: Shower Gel

| Ingredient | % |
|---|---|
| Aqua | ad 100 |
| Alcohol | 5.0 |
| Coco-Glucoside | 5.0 |
| Glycerin | 5.0 |
| SFX | 2.5 |
| Xanthan Gum | 2.5 |
| Disodium Cocoyl Glutamate | 2.0 |
| Citric Acid | ad pH 5.0 |
| Citrus Aurantifolia (Lime) Oil | 1.0 |
| Sodium Cocoyl Glutamate | 1.0 |
| Glyceryl Oleate | 1.0 |
| Sodium Lauroyl Lactylate | 0.7 |
| Limonene | 0.1 |
| Citral | 0.1 |
| Linalool | 0.1 |
| Dyes | q.s. |

Recipes 155: Oil Shower

| Ingredient | % |
|---|---|
| Aqua | ad 100 |
| Sodium Laureth Sulfate | 8.0 |
| Cocamidopropyl Betaine | 3.0 |
| SFX | 2.0 |
| Acrylates Copolymer | 2.0 |
| Tocopheryl Acetate | 0.5 |
| PEG-7 Glyceryl Cocoate | 0.5 |
| Disodium EDTA | 0.4 |
| Benzophenone-4 | 0.3 |
| PEG-40 Hydrogenated Castor Oil | 0.7 |
| Cellulose | 0.7 |
| Hydroxypropyl Methylcellulose | 0.7 |
| Sodium Chloride | 0.9 |
| Ethylhexylglycerin | 0.2 |
| Phenoxyethanol | 0.7 |
| Methylisothiazolinone | 0.3 |
| Sodium Benzoate | 0.3 |
| Sodium Hydroxide | 0.3 |
| Citric Acid | ad pH 5.5 |
| Parfum, Dyes | q.s. |

Recipes 156: Shower Gel

| Ingredient | % |
|---|---|
| Aqua | ad 100 |
| Ammonium Lauryl Sulfate | 6.0 |
| Coco-Betaine | 3.0 |
| SFX | 3.0 |
| Lauroyl/Myristoyl Methyl Glucamide | 1.5 |
| Citric Acid | ad pH 5.0 |
| Benzyl Alcohol, Glycerin, Benzoic Acid, Sorbic Acid | 1.0 |
| Parfum | 0.8 |
| Aloe Vera Leaf Extract | 0.1 |

Recipes 157: Showergel

| Ingredient | % |
|---|---|
| Aqua | ad 100 |
| SFX | 7.0 |
| Sesamum Indicum (Sesame) Seed Oil | 4.0 |
| Sucrose. Alcohol | 2.0 |
| Amino Acids | 1.0 |
| Glycerin | 1.0 |
| Carrageenan | 0.3 |
| Lavandula Officinalis Flower Oil | 0.3 |
| Xanthan Gum | 0.3 |
| Lactic Acid | 0.1 |

Recipes 158: Shower Scrub

| Ingredient | % |
|---|---|
| Aqua | ad 100 |
| Maris Sal | 15.0 |
| Sodium Laureth Sulfate | 9.0 |
| SFX | 2.0 |
| Sodium Cocoamphoacetate | 1.0 |
| Betaine | 1.0 |
| Citrus Limon (Lemon) Peel Powder | 0.5 |
| Lactic Acid | ad pH 5.5 |
| Parfum, Dyes | q s. |

Recipes 159: Cellulose Scrub

| Ingredient | % |
|---|---|
| Aqua | ad 100 |
| SFX | 7.0 |
| Coco Glucoside | 6.0 |
| Glycerin | 5.0 |
| Glyceryl Stearate | 5.0 |
| Citrus Aurantium Dulcis Fruit Water | 4.5 |
| Helianthus Annuus Hybrid Oil | 4.0 |
| Cellulose | 2.0 |
| Jojoba Esters | 2.0 |
| Cetyl Alcohol | 2.0 |
| Simmondsia Chinensis (Jojoba) Seed Oil | 2.0 |
| Olea Europaea (Olive) Fruit Oil | 2.0 |
| Hydrogenated Castor Oil | 0.8 |
| Prunus Armeniaca (Apricot) Kernel Oil | 0.5 |
| Leontopodium Alpinum Extract | 0.1 |
| Xanthan Gum | 0.3 |
| Benzyl Alcohol | 0.2 |
| Parfum | 0.1 |
| Dehydroacetic Acid | 0.1 |
| Citric acid | ad pH 5.0 |
| Lactic acid | 0.1 |
| Sodium Hydroxide | 0.1 |
| Parfum, Dyes | q.s. |

Recipes 160: Exfoliating Body Scrub

| Ingredient | % |
|---|---|
| Aqua | ad 100 |
| Coco-Glucoside | 7.0 |
| Alcohol | 6.0 |
| Glycerin | 5.0 |
| Glycine Soja Oil | 5.0 |
| Hydrated Silica | 4.0 |
| Xanthan Gum | 3.0 |
| SFX | 3.0 |
| Disodium/Sodium Cocoyl Glutamate | 1.0 |
| Coffea Arabica (Coffee) Seed Powder | 1.0 |
| Cocos Nucifera (Coconut) Shell Powder | 1.0 |
| Citric Acid | ad pH 5.2 |
| Glyceryl Oleate | 0.5 |
| Parfum | 0.1 |
| Preservative, Dyes | q.s. |

Recipes 161: Washing Powder

| Ingredient | % |
|---|---|
| Talc | 60.0 |
| Sodium Cocoyl Isethionate | 15.0 |
| Sodium C14-16 Olefin Sulfonate | 10.0 |
| Sodium Lauroyl Glutamate | 4.0 |
| SFX | 3.0 |
| Potassium Laurate | 2.0 |
| Sodium Myristoyl Glutamate | 1.5 |
| Chondrus Crispus | 1.5 |
| Isostearyl Alcohol | 1.0 |
| Glycerin | 1.0 |
| BHT | 0.4 |
| Butylene Glycol | 0.3 |
| Protease | 0.2 |
| Salicylic Acid | 0.1 |

Recipes 162: Washing Powder

| Ingredient | % |
|---|---|
| Hydrates Silica | ad 100 |
| Zea Mays Starch | 30.0 |
| Sodium Cocoyl Isethionate | 6.0 |
| Amylopektin | 5.0 |
| Sodium Lauryl Sulfate | 2.5 |
| Sodium Bicarbonate | 2.0 |
| Citric Acid | 1.5 |
| Sodium Lauroyl Glutamate | 1.0 |
| Sodium Palmitate | 1.0 |
| SFX | 1.0 |
| Diglycerin | 0.9 |
| Sodium Lauroyl Aspartate | 0.8 |
| Titanium Dioxide | 0.5 |
| Maltodextrin | 0.5 |
| Allantoin | 0.4 |
| Aqua | 0.3 |
| Butylene Glycol | 0.3 |
| Aloe Vera Leaf Extract | 0.1 |
| Citrus Limon (Lemon) Peel Powder | 0.1 |
| Parfum, Preservatives, Dyes | q.s. |

Recipes 163: Powder Cleanser

| Ingredient | % |
|---|---|
| Hydrated Silica | ad 100 |
| Tapioca Flour | 10.0 |
| Capryl/Capramidopropyl Betaine | 5.0 |
| Sodium Cocoamphoacetate | 5.0 |
| Helianthus Annuus (Sunflower) Seed Oil | 4.0 |
| Oleyl Erucate | 2.0 |
| Sucrose Cocoate | 2.0 |
| SFX | 2.0 |
| Coconut Acid | 1.0 |
| Citric Acid | ad pH 6.0 |
| Preservatives, parfum | q.s. |

Recipes 164: Powder Cleanser

| Ingredient | % |
|---|---|
| Talc | ad 100 |
| Distarch Phosphate | 10.0 |
| Disodium Lauryl Sulfosuccinate | 8.0 |
| Magnesium Aluminum Silicate | 5.0 |
| Oryza Sativa Powder | 5.0 |
| Sodium Lauroyl Glutamate | 2.0 |
| SFX | 2.0 |
| Glucose | 2.0 |
| Magnesium Oxide | 2.0 |
| Maltodextrin | 2.0 |
| Titanium Dioxide | 1.0 |
| Pumice | 0.5 |
| Chlorella Vulgaris Powder | 0.1 |

### Soap Recipes

Recipes 165: Gentle Liquid Soap

| Ingredient | % |
|---|---|
| Aqua | ad 100 |
| Sodium Laureth Sulfate | 8.5 |
| Cocamidopropyl Betaine | 3.0 |
| SFX | 2.5 |
| PEG-7 Glyceryl Cocoate | 2.5 |
| Cocamide DEA | 2.5 |
| Glycerin | 2.0 |
| Sorbitan Sesquicaprylate | 1.5 |
| C12-15 Pareth-12 | 1.2 |
| Sodium Benzoate | 1.0 |
| Potassium Sorbate | 1.0 |
| Citric Acid | ad pH 5.5 |
| PEG-200 Hydrogenated Glyceryl Palmate | 1.0 |
| Sodium Lactate | 1.0 |
| Sodium Chloride | 0.7 |
| Tetrasodium Glutamate Diacetate | 0.2 |
| Linalool | 0.1 |
| Limonene | 0.1 |
| Citronellol | 0.1 |
| Dyes | q.s. |

Recipes 166: Soap

| Ingredient | % |
|---|---|
| Aqua | ad 100 |
| Cocamidopropyl Betaine | 5.0 |
| SFX | 4.0 |
| Glycerin | 2.7 |
| PEG-7 Glyceryl Cocoate | 2.5 |
| Simmondsia Chinensis (Jojoba) Seed Oil | 2.5 |
| Glyceryl Glucoside | 2.3 |
| Mel | 2.0 |
| Sodium Laureth Sulfate | 3.0 |
| Sodium Chloride | 2.1 |
| Citric Acid | ad pH 5.0 |
| PEG-40 Hydrogenated Castor Oil | 1.8 |
| PEG-120 Methyl Glucose Dioleate | 1.7 |
| Trisodium EDTA | 1.0 |
| Glycol Distearate | 1.0 |
| Polyquaternium-10 | 0.3 |
| Laureth-10 | 0.7 |
| Benzophenone-4 | 0.5 |
| Cocamide MEA | 0.8 |
| Maltodextrin | 0.5 |
| Sodium Benzoate | 0.7 |
| Sodium Salicylate | 0.6 |
| Benzoic Acid | 0.4 |
| Linalool | 0.1 |
| Parfum, Dyes | q.s. |

Recipes 167: Caring Liquid Soap

| Ingredient | % |
|---|---|
| Aqua | ad 100 |
| Aloe Barbadensis Leaf Juice | 5.0 |
| Ammonium Lauryl Sulfate | 5.0 |
| Coco Glucoside | 4.0 |
| Cocamidopropyl Betaine | 4.0 |
| Sodium Cocoamphoacetate | 1.5 |
| SFX | 1.5 |
| Glycerin | 1.5 |
| Sodium Chloride | 1.5 |
| Citric Acid | ad pH 5.5 |
| Sodium Benzoate | 1.0 |
| Sodium Salicylate | 0.5 |
| Methylisothiazolinone | 0.3 |
| Linalool | 0.1 |
| Butylphenyl Methylpropional | 0.2 |
| Limonene | 0.1 |
| Dyes | q.s. |

Recipes 168: Cream Soap

| Ingredient | % |
|---|---|
| Aqua | ad 100 |
| Alcohol | 5.0 |
| Coco Glucoside | 5.0 |
| Glycerin | 5.0 |
| SFX | 2.5 |
| Disodium Cocoyl Glutamate | 2.0 |
| Xanthan Gum | 1.5 |
| Citric Acid | ad pH 5.5 |
| Malva Sylvestris Leaf Extract | 1.0 |
| Sodium Lauroyl Lactylate | 1.0 |
| Glyceryl Oleate | 1.0 |
| Sodium Cocoyl Glutamate | 0.7 |
| Linalool | 0.1 |
| Limonene | 0.1 |
| Dyes | q.s. |

Recipes 169: Liquid Soap

| Ingredient | % |
|---|---|
| Aqua | ad 100 |
| Sodium Laureth Sulfate | 5.0 |
| Sodium Chloride | 3.5 |
| Cocamidopropyl Betaine | 3.5 |
| Coco Glucoside | 3.0 |
| SFX | 2.0 |
| Glyceryl Oleate | 1.0 |
| Hibiscus Sabdariffa Flower Extract | 1.0 |
| Alcohol | 1.0 |
| Sodium Lactate | 1.0 |
| Citric Acid | ad pH 5.0 |
| Sodium Benzoate | 0.7 |
| Potassium Sorbate | 0.1 |
| Parfum, Dyes | q.s. |

Recipes 170: Liquid Soap

| Ingredient | % |
|---|---|
| Aqua | ad 100 |
| Glycerin | 7.0 |
| Alcohol | 4.0 |
| Sodium Coco Sulfate | 3.0 |
| Lauryl Glucoside | 2.5 |
| SFX | 2.0 |
| Xanthan Gum | 1.5 |
| Mangifera Indica (Mango) Fruit Extract | 0.7 |
| Limonene | 0.1 |
| Linalool | 0.1 |
| Dyes | q.s. |

Recipes 171: Liquid Soap

| Ingredient | % |
|---|---|
| Aqua | ad 100 |
| Sodium Lauryl Sulfate | 6.0 |
| Cocamidopropyl Betaine | 3.0 |
| SFX | 3.0 |
| Triethylcitrate; Caprylyl Glycol; Benzoic Acid | 1.0 |
| Lactic Acid | ad pH 5.0 |
| Chamomilla Recutita Extract | 0.5 |
| Sodium Chloride | 0.9 |
| Sodium Lactate | 0.7 |
| Styrene/Acrylates Copolymer | 0.5 |
| Glycerin | 0.5 |
| Polyquaternium-7 | 0.3 |
| Hexyl Cinnamal | 0.1 |
| Sorbitol | 0.2 |
| Trisodium Ethylenediamine Disuccinate | 0.2 |
| Propylene Glycol | 0.3 |
| PEG-40 Hydrogenated Castor Oil | 0.9 |
| 1,2-Hexandiol | 0.5 |
| Parfum, Dyes | q.s. |

Recipes 172: Cream Soap

| Ingredient | % |
|---|---|
| Aqua | ad 100 |
| Sodium Laureth Sulfate | 8.0 |
| Cocamidopropyl Betaine | 3.0 |
| Sodium Chloride | 2.6 |
| SFX | 2.5 |
| Polyquaternium-7 | 0.4 |
| Glycerin | 0.9 |
| Glycol Distearate | 0.7 |
| Laureth-4 | 0.5 |
| Laureth-2 | 0.8 |
| PEG-55 Propylene Glycol Oleate | 0.4 |
| Propylene Glycol | 0.5 |
| Citric Acid | ad pH 5.5 |
| Hexyl Cinnamal | 0.1 |
| Benzyl Salicylate | 0.1 |
| Linalool | 0.1 |
| Limonene | 0.1 |
| Butylphenyl Methylpropional | 0.1 |
| Sodium Benzoate | 0.1 |
| Dyes | q.s. |

Recipes 173: Jelly Soap

| Ingredient | % |
|---|---|
| Glycerin | 50.0 |
| Decyl Glucoside | 5.0 |
| SFX | 2.5 |
| Chondrus Crispus Powder | 2.0 |
| Methylpropanediol | 1.0 |
| Caprylyl Glycol | 1.0 |
| Phenylpropanol | 0.5 |
| Parfum | 0.2 |
| Helianthus Annuus (Sunflower) Seed Oil | 0.2 |
| Hydrogenated Coconut Oil | 0.2 |
| Olea Europea (Olive) Fruti Oil | 0.2 |
| Oryza Sativa (Rice) Bran Oil | 0.2 |
| Vitis Vinifera (Grape) Seed Oil | 0.1 |
| Prunus Amygdalus Dulcis (Sweet Almond) Oil | 0.1 |
| Aqua | ad 100 |
| Sodium Benzoate | 0.1 |
| Citric Acid | 0.1 |

Recipes 174: Soap

| Ingredient | % |
|---|---|
| Talc | 65.0 |
| SFX | 10.0 |
| Corn (Zea Mays) Flour | 9.0 |
| Glycerin | 7.0 |
| Sodium Laureth Sulfate | 4.5 |
| Citrus Aurantium Bergamia Fruit Oil | 3.5 |
| Benzyl Benzoate | 0.5 |
| Butylphenyl Methylpropional | 0.2 |
| Parfum | 0.2 |

Recipes 175: Soap

| Ingredient | % |
|---|---|
| Aqua | ad 100 |
| SFX | 6.0 |
| Propylene Glycol | 5.0 |
| Helianthus Annuus Seed Oil | 4.0 |
| Cocos Nucifera Oil | 4.0 |
| Titanium Dioxide | 0.2 |
| Sodium Hydroxide | 0.2 |
| Sodium Coco-sulfate | 0.2 |
| Parfum | 0.1 |
| Sodium Chloride | 0.1 |
| L-limonene | 0.1 |

Recipes 176: Painting Soap

| Ingredient | % |
|---|---|
| Aqua | ad 100 |
| Mica | 7.0 |
| Glycine Soja Oil | 5.0 |
| Glycerin | 4.0 |
| Alcohol | 3.0 |
| Cocamidopropyl Betaine | 3.0 |
| SFX | 2.0 |
| Cetearyl Alcohol | 1.5 |
| Myristyl Alcohol | 1.0 |
| Glyceryl Stearate Citrate | 1.0 |
| Parfum | 0.4 |
| Xanthan Gum | 0.3 |
| Helianthus Annuus Seed Oil | 0.2 |
| Simmondsia Chinensis Seed Oil | 0.2 |
| Rubus Idaeus Fruit Extract | 0.2 |
| Tocopherol | 0.1 |

Recipes 177: Painting Soap

| Ingredient | % |
|---|---|
| Aqua | ad 100 |
| Glycerin | 10.0 |
| Zea Starch | 9.0 |
| Lauryl Glucoside | 4.0 |
| Methylpropanediol | 3.0 |
| Stearyl Alcohol | 3.0 |
| Cocamidopropyl Betaine | 2.0 |
| SFX | 2.0 |
| Glycine Soja Oil | 1.0 |
| Tocopherol | 0.5 |
| Coco-Glucoside | 0.4 |
| Panthenol | 0.2 |
| Glycerin Oleate | 0.2 |
| Caprylyl Glycol | 0.2 |
| Phenlypropanol | 0.2 |
| Sodium Hydroxide | 0.1 |
| Sodium Chloride | 0.1 |
| Xanthan Gum | 0.1 |
| Citric Acid | 0.1 |
| Parfum | 0.1 |
| Beta Carotene | 0.1 |
| Dyes | q.s. |

### Bath Recipes

Recipes 178: Bath

| Ingredient | % |
|---|---|
| Aqua | ad 100 |
| PEG-80 Sorbitan Laurate | 4.0 |
| Cocamidopropyl Betaine | 4.0 |
| Sodium Laureth Sulfate | 4.0 |
| SFX | 2.0 |
| Polysorbate 20 | 2.0 |
| Sodium Chloride | 1.5 |
| Citric Acid | ad pH 5.0 |
| Sodium Lauroamphoacetate | 1.0 |
| PEG-150 Distearate | 1.0 |
| Disodium Lauroamphodiacetate | 1.0 |
| Sodium Benzoate | 0.5 |
| Parfum, Dyes | q.s. |

Recipes 179: Creme Oil Bath

| Ingredient | % |
|---|---|
| Aqua | ad 100 |
| Sodium Laureth Sulfate | 5.0 |
| Cocamidopropyl Betaine | 3.0 |
| Decyl Glucoside | 2.8 |
| SFX | 2.5 |
| Glycerin | 2.5 |
| Glyceryl Glucoside | 2.0 |
| Diamond Powder | 2.0 |
| Helianthus Annuus (Sunflower) Seed Oil | 1.7 |
| Sodium Chloride | 1.5 |
| Glycol Distearate | 1.5 |
| PEG-40 Hydrogenated Castor Oil | 1.5 |
| Citric Acid | ad pH 5.5 |
| PEG-90 Glyceryl Isostearate | 1.2 |
| Laureth-4 | 1.0 |
| PEG-200 Hydrogenated Glyceryl Palmate | 1.0 |
| PEG-7 Glyceryl Cocoate | 1.0 |
| Benzophenone-4 | 0.9 |
| Laureth-2 | 0.7 |
| Sodium Benzoate | 0.7 |
| Butylphenyl Methylpropional | 0.1 |
| Linalool | 0.1 |
| Limonene | 0.1 |
| alpha-Isomethyl Ionone | 0.1 |
| Dyes | q.s. |

Recipes 180: Relaxing Good Sleep Bath

| Ingredient | % |
|---|---|
| Aqua | ad 100 |
| Cocamidopropyl Betaine | 3.0 |
| Coco Glucoside | 2.5 |
| Sodium Chloride | 2.5 |
| Disodium Lauryl Sulfosuccinate | 2.0 |
| SFX | 2.0 |
| Glycerin | 1.7 |
| Sodium PCA | 0.5 |
| Glyceryl Oleate | 0.4 |
| Sodium Hydroxide | 0.3 |
| Citric Acid | ad pH 5.5 |
| Linalool | 0.1 |
| Limonene | 0.1 |
| Geraniol | 0.1 |
| Dyes | q.s. |

Recipes 181: Pampering Oil Bath

| Ingredient | % |
|---|---|
| Aqua | ad 100 |
| Glycine Soja Oil | 20.0 |
| SFX | 5.0 |
| Polyglyceryl-3 Palmitate | 4.5 |
| Glyceryl Caprylate | 1.5 |
| Simmondsia Chinensis Oil | 1.5 |
| Prunus Amygdalus Dulcis (Sweet Almond) Oil | 1.0 |
| Triticum Vulgare Germ Oil | 1.0 |
| Tocopherol | 0.2 |
| Limonene | 0.1 |
| Linalool | 0.1 |
| p-Anisic Acid | 0.1 |
| Dyes | q.s. |

Recipes 182: Aroma Bath

| Ingredient | % |
|---|---|
| Aqua | ad 100 |
| Alcohol | 5.0 |
| Coco Glucoside | 5.0 |
| Sodium Coco Sulfate | 4.0 |
| SFX | 2.5 |
| Xanthan Gum | 1.9 |
| Glycerin | 1.8 |
| Sodium Cocoyl Glutamate | 1.5 |
| Disodium Cocoyl Glutamate | 1.5 |
| Melissa Officinalis Extract | 1.0 |
| Lavandula Angustifolia Oil | 1.0 |
| Linalool | 0.1 |
| Limonene | 0.1 |
| Geraniol | 0.1 |
| Dyes | q.s. |

Recipes 183: Bath Bomb

| Ingredient | % |
|---|---|
| Sodium Bicarbonate | 70.0 |
| Citric Acid | 8.0 |
| Citrus Nobilis Oil | 6.0 |
| Potassium Tartrate | 6.0 |
| Sodium Laureth Sulfate | 4.0 |
| SFX | 4.0 |
| Betaine | 1.5 |
| Parfum | 0.5 |
| Dyes | q.s. |

Recipes 184: Bath Bomb

| Ingredient | % |
|---|---|
| Sodium Bicarbonate | 67.0 |
| Citric Acid | 9.0 |
| Zea Mays Starch | 8.0 |
| Butyrospermum Parkii Butter | 5.0 |
| Sodium Lauryl Sulfoacetate | 4.0 |
| SFX | 4.0 |
| Theobroma Cacao Seed Butter | 2.0 |
| Theobroma Cacao (Cocoa) Fruit Powder | 1.0 |
| Parfum, Dyes | q.s. |

Recipes 185: Bath Salt

| Ingredient | % |
|---|---|
| Maris Sal | ad 100 |
| Parfum | 2.0 |
| SFX | 1.0 |
| Silica | 0.5 |
| Simmondsia Chinensis Seed Oil | 0.1 |
| Preservative, Dyes | q.s. |

Recipes 186: Bath Salt

| Ingredient | % |
|---|---|
| Maris Sal | ad 100 |
| Rosmarinus Officinalis Leaf Oil | 3.0 |
| Parfum | 1.5 |
| Polysorbate 20 | 1.5 |
| SFX | 1.5 |
| Preservative, Dyes | q.s. |

Recipes 187: Shower Gel and Shampoo

| Ingredient | % |
|---|---|
| Aqua | ad 100 |
| Coco Glucoside | 5.0 |
| Sodium Coco Sulfate | 3.0 |
| Cocamidopropyl Hydroxysultaine | 2.5 |
| SFX | 2.0 |
| Lauryl Glucoside | 2.0 |
| Glyceryl Oleate | 1.8 |
| Inulin | 1.5 |
| Lecithin | 1.5 |
| Polyquaternium-39 | 1.5 |
| Citric Acid | ad pH 5.0 |
| Styrene/Acrylates Copolymer | 1.0 |
| Sodium Chloride | 1.0 |
| Denatonium Benzoate | 0.5 |
| Hydrogenated Palm Glycerides Citrate | 0.7 |
| Tocopherol | 0.3 |
| Sodium Benzoate | 0.7 |
| Phenoxyethanol | 0.3 |
| Parfum, Dyes | q.s. |

Recipes 188: Shower, Shampoo and Shave

| Ingredient | % |
|---|---|
| Aqua | ad 100 |
| Cocamidopropyl Betaine | 5.0 |
| Sodium Laureth Sulfate | 4.0 |
| PEG-7 Glyceryl Cocoate | 4.0 |
| Sodium Chloride | 3.5 |
| SFX | 2.0 |
| PEG-3 Distearate | 3.0 |
| Glycerin | 2.5 |
| Glyceryl Glucoside | 1.2 |
| Helianthus Annuus (Sunflower) Seed Oil | 1.0 |
| Persea Gratissima (Avocado) Oil | 1.0 |
| Polyquaternium-7 | 1.0 |
| PEG-90m | 1.0 |
| PEG-40 Hydrogenated Castor Oil | 1.0 |
| Citric Acid | ad pH 5.0 |
| Benzophenone-4 | 0.5 |
| PEG-200 Hydrogenated Glyceryl | 0.5 |
| Palmate | |
| Silica | 0.7 |
| Sodium Benzoate | 0.6 |
| Methylparaben | 0.4 |
| Propylparaben | 0.4 |
| Limonene | 0.1 |
| Butylphenyl Methylpropional | 0.1 |
| Benzyl Alcohol | 0.1 |
| Dyes | q.s. |

Recipes 189: 2-in-1 Body Wash and Shampoo

| Ingredient | % |
|---|---|
| Aqua | ad 100 |
| Sodium Laureth Sulfate | 8.5 |
| Glycerin | 2.5 |
| Sodium Cocoyl Isethionate | 2.5 |
| Ammonium Lauryl Sulfate | 2.5 |
| Lauryl Betaine | 2.5 |
| Lauryl Glucoside | 1.5 |
| Sodium Chloride | 1.0 |
| SFX | 1.0 |
| Polyquaternium-39 | 0.5 |
| Polyquaternium-7 | 0.5 |
| Panthenol | 0.1 |
| Citric Acid | ad pH 5.5 |
| Sodium Citrate | 0.2 |
| Propylene Glycol | 0.3 |
| PEG-55 Propylene Glycol Oleate | 0.7 |
| Styrene/Acrylates Copolymer | 0.2 |
| Phenoxyethanol | 0.7 |
| Sodium Benzoate | 0.3 |
| Tetrasodium EDTA | 0.1 |
| Parfum, Dyes | q.s. |

Recipes 190: 2-in-1 Bubble Bath and Wash

| Ingredient | % |
|---|---|
| Aqua | ad 100 |
| Cocamidopropyl Betaine | 4.0 |
| Glycerin | 4.0 |
| Sodium Lauroamphodiacetate | 2.0 |
| Sodium Chloride | 2.0 |
| Coco Glucoside | 1.5 |
| Hydroxypropyl Starch Phosphate | 1.0 |
| Disodium Lauroamphodiacetate | 1.0 |
| SFX | 1.0 |
| Lauric Acid | 1.0 |
| Acrylates/C10-30 Alkyl Acrylate Crosspolymer | 1.0 |
| Polyquaternium-7 | 1.0 |
| Styrene/Acrylates Copolymer | 0.5 |
| Citric Acid | ad pH 5.5 |
| Sodium Hydroxide | 0.3 |
| Sodium Benzoate | 0.2 |
| Benzoic Acid | 0.1 |
| Parfum, Dyes | q.s. |

Recipes 191: Body and Face Wash

| Ingredient | % |
|---|---|
| Aqua | ad 100 |
| Sodium Laureth Sulfate | 4.5 |
| Petrolatum | 3.0 |
| Acrylates Copolymer | 2.5 |
| Cocamide MEA | 2.5 |
| SFX | 2.0 |
| PPG-9 | 2.0 |
| Guar hydroxypropyltrimonium chloride | 0.2 |
| Menthol | 0.2 |
| DMDM Hydantoin | 0.1 |
| Tetrasodium EDTA | 0.2 |
| Methylisothiazolinone | 0.1 |
| BHT | 0.1 |
| Parfum, Dyes | q.s. |

Recipes 192: Hair and Body Shampoo

| Ingredient | % |
|---|---|
| Aqua | ad 100 |
| Sodium Laureth Sulfate | 10.0 |
| Sodium Chloride | 4.0 |
| Glycerin | 3.5 |
| Coco-Betaine | 2.0 |
| SFX | 2.0 |
| Niacinamide | 0.1 |
| Saccharum Officinarum (Sugar Cane) Extract | 1.0 |
| Sodium Benzoate | 1.0 |
| Sodium Hydroxide | 0.3 |
| PPG-5-Ceteth-20 | 1.0 |
| Polyquaternium-10 | 0.7 |
| Salicylic Acid | 0.5 |
| Limonene | 0.1 |
| Linalool | 0.2 |
| Benzyl Salicylate | 0.1 |
| Pyrus Malus (Apple Fruit) Extract | 0.3 |
| Pyridoxine HCl | 0.2 |
| Citric Acid | ad pH 5.0 |
| Taurine | 0.1 |
| Dyes | q.s. |

Recipes 193: After-Sun Hair and Body Shampoo

| Ingredient | % |
|---|---|
| Aqua | ad 100 |
| Sodium Laureth Sulfate | 7.0 |
| Cocamidopropyl Betaine | 5.0 |
| PEG-18 Glyceryl Oleate/Cocoate | 2.5 |
| SFX | 2.0 |
| Glycol Distearate | 2.0 |
| Coco Glucoside | 2.0 |
| Laureth-4 | 1.5 |
| Sodium Chloride | 0.9 |
| Panthenol | 0.1 |
| Sodium PCA | 0.7 |
| Polyquaternium-10 | 0.3 |
| Octyldodecyl PCA | 0.2 |
| Glycerin | 0.9 |
| PPG-9 | 0.7 |
| Citric Acid | ad pH 5.5 |
| Tetrasodium EDTA | 0.3 |
| Quaternium-80 | 0.2 |
| Propylene Glycol | 0.3 |
| Isopropyl Alcohol | 0.3 |
| Sodium Benzoate | 0.7 |
| Limonene | 0.1 |
| Linalool | 0.1 |
| Benzyl Salicylate | 0.1 |
| Butylphenyl Methylpropional | 0.1 |
| Ceramide NP | 0.05 |
| Dyes | q.s. |

Recipes 194: Hair and Body Shampoo & Shower Gel

| Ingredient | % |
|---|---|
| Aqua | ad 100 |
| Sodium Laureth Sulfate | 7.0 |
| Sodium Chloride | 3.8 |
| Cocamidopropyl Betaine | 3.5 |
| SFX | 3.0 |
| Panthenol | 0.3 |
| Hydrolyzed Keratin | 0.7 |
| Taurine | 0.5 |
| Simmondsia Chinensis Seed Oil | 0.5 |
| Disodium Cocoamphodiacetate | 0.5 |
| Citric Acid | ad pH 5.0 |
| Sodium Benzoate | 0.7 |
| Cocamide MEA | 0.4 |
| PEG-7 Glyceryl Cocoate | 0.3 |
| Polyquaternium-10 | 0.2 |
| Propylene Glycol | 0.3 |
| PEG-40 Hydrogenated Castor Oil | 0.4 |
| Linalool | 0.2 |
| Geraniol | 0.1 |
| Butylphenyl Methylpropional | 0.1 |
| Dyes | q.s. |

### Facial Cleansing Recipes

Recipes 195: Eye Make-Up Remover

| Ingredient | % |
|---|---|
| Aqua | ad 100 |
| Butylene Glycol | 6.0 |
| Propanediol | 2.0 |
| Decyl Glucoside | 1.0 |
| SFX | 1.0 |
| Glycerin | 1.0 |
| Aloe Barbadensis Leaf Extract | 0.5 |
| Citric Acid | ad pH 7 |
| Bioflavonoids | 0.1 |
| Pantolactone | 0.1 |

Recipes 196: Eye Make-Up Remover

| Ingredient | % |
|---|---|
| Aqua | ad 100 |
| Cyclopentasiloxane | 8.0 |
| Glycerin | 6.0 |
| Isohexadecane | 6.0 |
| Butylene Glycol | 4.0 |
| Panthenol | 3.0 |
| SFX | 3.0 |
| Sodium Chloride | 2.0 |
| Disodium EDTA | 1.0 |
| Sodium Hydroxide | 0.2 |
| Citric Acid | ad pH 7 |
| Phenoxyethanol | 0.2 |
| Methylparaben | 0.1 |

Recipes 197: Micellar Lotion

| Ingredient | % |
|---|---|
| Aqua | ad 100 |
| Poloxamer 124 | 2.0 |
| SFX | 2.0 |
| Glyceryl Glucoside | 1.5 |
| Glycerin | 1.0 |
| Polyglyceryl-10 Laurate | 1.0 |
| 1,2-Hexandiol | 1.0 |
| Decyl Glucoside | 0.5 |
| Arginine HCl | 0.3 |
| Sodium Cocoamphoacetate | 0.5 |
| Trisodium EDTA | 0.2 |
| Citric Acid | ad pH 6.5 |
| Phenoxyethanol | 0.1 |
| Sodium Hyaluronate | 0.05 |

Recipes 198: Waterproof Eye Make-Up Removal

| Ingredient | % |
|---|---|
| Aqua | ad 100 |
| Aloe Barbadensis Leaf Juice | 5.0 |
| Sorbitol | 3.0 |
| Glycerin | 3.0 |
| Panthenol | 2.2 |
| Sodium Lauryl Glucose Carboxylate | 2.0 |
| Biosaccharide Gum-1 | 1.5 |
| Lauryl Glucoside | 1.0 |
| SFX | 0.5 |
| Allantoin | 0.2 |
| Phenoxyethanol | 0.2 |
| Sodium Benzoate | 0.2 |
| Sorbic Acid | q.s. |
| Citric Acid | ad pH 6 |

Recipes 199: Waterproof Eye Make-Up Removal

| Ingredient | % |
|---|---|
| Aqua | ad 100 |
| Isododecane | 6.0 |
| Isopropyl Palmitate | 4.0 |
| Dimethicone | 3.0 |
| Sodium Ascorbyl Phosphate | 2.0 |
| Sodium Chloride | 2.0 |
| SFX | 1.5 |
| Caprylyl/Capryl Glucoside | 1.5 |
| Trisodium EDTA | 1.0 |
| Citric Acid | ad pH 7 |
| Sodium Hydroxide | 0.2 |
| Phenoxyethanol | 0.2 |
| Benzethonium Chloride | 0.2 |

Recipes 200: Micellar Gel

| Ingredient | % |
|---|---|
| Aqua | ad 100 |
| Propanediol | 5.0 |
| SFX | 3.0 |
| Glycerin, Aqua, Sodium Levulinate, Sodium Anisate | 3.0 |
| Polyglyceryl-4 Caprate | 2.0 |
| Xanthan Gum | 1.5 |
| Lactobacillus Ferment | 0.05 |
| Lactic Acid | ad pH 5.5 |

Recipes 201: Micellar Gel

| Ingredient | % |
|---|---|
| Aqua | ad 100 |
| Propylene Glycol | 4.0 |
| Glycerin | 4.0 |
| Poloxamer 124 | 2.5 |
| SFX | 2.5 |
| 1,2-Hexandiol | 2.0 |
| Pentylene Glycol | 1.0 |
| Acrylates/C10-30 Alkyl Acrylate Crosspolymer | 0.5 |
| Sodium Hyaluronate | 0.2 |
| Trisodium EDTA | 0.1 |
| Sodium Hydroxide | ad pH 6 |
| Cetrimonium Chloride | 0.1 |
| Phenoxyethanol | 0.1 |

Recipes 202: Micellar Make-Up Remover

| Ingredient | % |
|---|---|
| Aqua | ad 100 |
| Aloe Barbadensis Leaf Juice | 4.0 |
| Caprylyl/Capryl Glucoside | 2.0 |
| SFX | 1.5 |
| Betaine | 1.0 |
| Glycerin | 1.0 |
| Benzyl Alcohol, Caprylyl Glycol, Benzoic Acid | 1.0 |
| Decyl Glucoside | 0.5 |
| Parfum | q. s. |
| Citric Acid | ad pH 7.0 |

Recipes 203: Facial Cleansing Foam

| Ingredient | % |
|---|---|
| Aqua | ad 100 |
| Glycine Soja Oil | 5.0 |
| Coco-Glucoside | 4.0 |
| Glycerin | 3.5 |
| SFX | 3.0 |
| Alcohol Denat. | 2.0 |
| Sodium Coco-Sulfate | 1.5 |
| Sodium/Disodium Cocoyl Glutamate | 1.0 |
| Glyceryl Oleate | 0.5 |
| Hamamelis Virginiana Leaf Extract | 0.5 |
| Tocopherol | 0.1 |
| Helianthus Annuus Seed Oil | 0.1 |
| Lactic Acid | ad pH 6.5 |
| Preservative | q. s. |

Recipes 204: Facial cleansing Foam

| Ingredient | % |
|---|---|
| Aqua | ad 100 |
| Sodium/Disodium Cocoyl Glutamate | 4.0 |
| SFX | 4.0 |
| Sodium Lauryl Sulfate | 3.0 |
| Parfum | 1.0 |
| Benzyl Alcohol, Caprylyl Glycol, Benzoic Acid | 1.0 |
| Glycerin | 0.8 |
| Sodium Lauryl Glucose Carboxylate | 0.7 |
| Sodium Cottonseedamphoacetate | 0.7 |
| Lauryl Glucoside | 0.5 |
| Citric Acid | ad pH 7.0 |
| Capryloyl Glycine | 0.2 |
| Sodium Hydroxide | 0.2 |
| 1,2-Hexandiol | 0.2 |
| Allantoin | 0.1 |
| Hydroxypropyl Guar Hydroxypropyltrimonium Chloride | 0.1 |
| Tetrasodium EDTA | 0.1 |
| Dehydroacetic Acid | 0.1 |
| Sorbic Acid | 0.1 |
| Tropolone | 0.1 |

Recipes 205: Cleansing Foam

| Ingredient | % |
|---|---|
| Aqua | ad 100 |
| Coco-Glucoside | 2.0 |
| Glycerin | 1.5 |
| Sodium Coco-Sulfate | 1.5 |
| Glyceryl Oleate | 1.0 |
| Decyl Glucoside | 0.5 |
| Sodium Benzoate | 0.5 |
| Citric Acid | ad pH 5.0 |
| Panthenol | 0.4 |
| SFX | 0.2 |
| Albumen | 0.1 |
| Potassium Sorbate | 0.1 |
| Tocopherol | 0.1 |
| Hydrogenated Palm Glycerides Citrate | 0.1 |

Recipes 206: Cleansing Foam

| Ingredient | % |
|---|---|
| Aqua | ad 100 |
| Aloe Barbadensis Leaf Juice | 28.0 |
| Glycerin | 7.0 |
| Decyl Glucoside | 5.0 |
| Ammonium Lauryl Sulfate | 2.0 |
| Benzyl Alcohol, Caprylyl Glocol, Benzoic Acid | 2.0 |
| Parfum | 0.8 |
| SFX | 0.1 |
| Phytic Acid | 0.1 |
| Sodium Hydroxide | ad pH 7.0 |
| Gluconolactone | 0.1 |

Recipes 207: Cleansing Foam

| Ingredient | % |
|---|---|
| Aqua | ad 100 |
| Glycerin | 3.0 |
| Decyl Glucoside | 3.0 |
| Pentylene Glycol | 2.0 |
| SFX | 1.0 |
| Citric Acid | ad pH 6.0 |
| Papain | 0.1 |

Recipes 208: Pump Foam for Facial Cleansing

| Ingredient | % |
|---|---|
| Aqua | ad 100 |
| Sorbitol | 9.0 |
| Glycerin | 5.0 |
| Decyl Glucoside | 4.0 |
| Disodium Cocoyl Glutamate | 4.0 |
| Glyceryl Glucoside | 2.0 |
| SFX | 2.0 |
| Propylene Glycol | 2.0 |
| Prunus Amygdalus Dulcis Oil | 2.0 |
| PEG-40 Hydrogenated Castor Oil | 1.5 |
| Sodium Sulfate | 0.5 |
| Citric Acid | ad pH |
| Polyquaternium-10 | 0.5 |
| Sodium Benzoate | 0.2 |
| Aminopeptidase | 0.1 |
| Panthenol | 0.1 |

Recipes 209: Pump Foam for Facial Cleansing

| Ingredient | % |
|---|---|
| Aqua | ad 100 |
| Sucrose | 12.0 |
| Glycerin | 6.0 |
| Rosa Damascena Flower Water | 5.0 |
| Coco-Glucoside | 4.5 |
| SFX | 2.5 |
| Aqua, Sodium Levulinate, Potassium Sorbate | 2.0 |
| Sodium Cocoyl Glutamate | 3.0 |
| Polyglyceryl-4 Caprate | 3.0 |
| Betaine | 1.0 |
| Citric Acid | ad pH 5.0 |
| Parfum | 0.8 |
| Sodium Chloride | 0.5 |

Recipes 210: Micellar Water

| Ingredient | % |
|---|---|
| Aqua | ad 100 |
| PEG-6 Caprylic/capric Glycerides | 3.0 |
| Polysorbate 20 | 2.0 |
| SFX | 0.8 |
| Butylene Glycol | 0.5 |
| PEG-8 | 0.4 |
| Poloxamer 407 | 0.4 |
| Disodium EDTA | 0.3 |
| Citric Acid | ad pH 6.5 |
| BHT | 0.2 |
| Sodium Benzoate | 0.2 |

Recipes 211: Refreshing Micellar Water

| Ingredient | % |
|---|---|
| Aqua | ad 100 |
| Methylpropanediol | 3.0 |
| Propylene Glycol | 2.5 |
| Glycerin | 2.5 |
| Sodium Cocoamphoacetate | 2.0 |
| Parfum | 0.8 |
| SFX | 0.5 |
| Sodium Salicylate | 0.5 |
| Sodium Benzoate | 0.5 |
| Citric Acid | ad pH |
| Sodium Chloride | 0.2 |
| Tetrasodium EDTA | 0.1 |

Recipes 212: Oil-infused Micellar Water

| Ingredient | % |
|---|---|
| Aqua | ad 100 |
| Isododecane | 7.5 |
| Dimethicone | 7.5 |
| Dicaprylyl Ether | 5.0 |
| Hexylene Glycol | 1.4 |
| Propanediol | 1.0 |
| SFX | 1.0 |
| Sodium Chloride | 1.0 |
| Trisiloxane | 0.8 |
| Alcohol Denat. | 0.8 |
| Glycerin | 0.8 |
| Propylene Glycol Dicaprylate/Dicaprate | 0.8 |
| Disodium EDTA | 0.5 |
| Hydroxyethylcellulose | 0.5 |
| Phenoxyethanol, Benzoic Acid | 0.5 |
| Decyl Glucoside | 0.5 |
| Caprylyl Glycol | 0.5 |
| Sodium Citrate | 0.3 |
| Citric Acid | ad pH 6 |
| Panthenol | 0.2 |
| Benzophenone-3 | 0.1 |
| Tocopheryl Acetate | 0.1 |
| Tocopherol | 0.1 |
| Zea Mays Oil | 0.1 |
| Chlorphenesin | 0.1 |

Recipes 213: Facial Tonic

| Ingredient | % |
|---|---|
| Aqua | ad 100 |
| Glycerin. | 2.5 |
| Propanediol | 2.0 |
| Arginine | 1.0 |
| SFX | 1.0 |
| Salicylic Acid | 1.0 |
| Coco-Betaine | 1.0 |
| Caprylyl/Capryl Glucoside | 0.8 |
| Disodium Cocoyl Glutamate | 0.8 |
| Xanthan Gum | 0.5 |
| Phytic Acid | 0.5 |
| Sodium Chloride | 0.5 |
| Sodium Cocoyl Glutamate | 0.4 |
| Citric Acid | ad pH 5.5 |

Recipes 214: Facial Tonic

| Ingredient | % |
|---|---|
| Aqua | ad 100 |
| Glycerin | 5.0 |
| Caprylyl/Capryl Glucoside | 2.0 |
| SFX | 1.5 |
| Glyceryl Caprylate | 1.5 |
| Parfum | 1.0 |
| Sodium Levulinate | 1.0 |
| Sodium Anisate | 1.0 |
| Sodium Salicylate | 1.0 |
| Sodium Cocoyl Glutamate | 0.1 |
| Polyglyceryl-6 Oleate | 0.1 |
| Disodium Cocoyl Glutamate | 0.1 |
| Citric Acid | ad pH 5.5 |
| Sodium Surfactin | 0.05 |

Recipes 215: Facial Tonic

| Ingredient | % |
|---|---|
| Aqua | ad 100 |
| PEG-6 Caprylic/Capric Glycerides | 4.0 |
| SFX | 3.0 |
| Fructooligosaccharides | 1.0 |
| Mannitol | 1.0 |
| Xylitol | 1.0 |
| Propylene Glycol | 1.0 |
| Cetrimonium Bromide | 1.0 |
| Disodium EDTA | 0.4 |
| Rhamnose | 0.1 |

Recipes 216: Micellar Water

| Ingredient | % |
|---|---|
| Aqua | ad 100 |
| Glycerin | 5.0 |
| Polyglyceryl-4 Caprate | 5.0 |
| Betaine | 3.0 |
| SFX | 2.0 |
| Sodium Levulinate | 2.0 |
| Lactobacillus Ferment | 0.2 |
| Sodium Benzoate | 0.2 |
| Sodium Hydroxide | 0.2 |
| Parfum | 0.1 |
| Lactic Acid | ad pH 5.8 |

Recipes 217: Micellar Water

| Ingredient | % |
|---|---|
| Aqua | ad 100 |
| Glycerin | 6.0 |
| Poloxamer 184 | 2.5 |
| Phenoxyethanol | 1.0 |
| PEG-40 Hydrogenated Castor Oil | 1.0 |
| Lactobacillus Extract, Propylene Glycol | 0.8 |
| SFX | 0.5 |
| Parfum | 0.5 |
| Citric Acid | ad pH 6.4 |
| Sodium Hydroxide | 0.2 |
| Disodium EDTA | 0.1 |

Recipes 218: Micellar Water

| Ingredient | % |
|---|---|
| Aqua | ad 100 |
| SFX | 5.0 |
| PEG-40 Hydrogenated Castor Oil | 2.5 |
| Glycerin | 2.5 |
| Prunus Amygdalus Dulcis Oil | 1.0 |
| Sorbitol | 1.0 |
| Decyl Glucoside | 1.0 |
| 1,2-Hexandiol | 0.1 |
| Glyceryl Glucoside | 0.5 |
| Poloxamer 124 | 0.5 |
| Propylene Glycol | 0.5 |
| Disodium Cocoyl Glutamate | 0.5 |
| Sodium Chloride | 0.2 |
| Trisodium EDTA | 0.2 |
| Polyquaternium-10 | 0.2 |
| Citric Acid | ad pH 6.2 |
| Sodium Acetate | 0.1 |
| Phenoxyethanol | 0.1 |

Recipes 219: Micellar Water

| Ingredient | % |
|---|---|
| Aqua | ad 100 |
| SFX | 5.0 |
| Glycerin | 4.0 |
| Coco-Glucoside | 2.0 |
| Polysorbate 80 | 0.2 |
| Calendula Officinalis Flower Extract | 2.0 |
| Caprylyl Glycol | 1.5 |
| Citric Acid | ad pH |
| Sodium Benzoate | 0.9 |
| Parfum | 0.6 |
| Isoleucine | 0.2 |
| Panthenol | 0.2 |
| PPG-1-PEG-9 Lauryl Glycol Ether | 0.2 |
| Propylene Glycol | 0.2 |
| Phytosphingosine | 0.1 |
| Sodium Chloride | 0.2 |

Recipes 220: Micellar Water

| Ingredient | % |
|---|---|
| Aqua | ad 100 |
| Hexylene Glycol | 1.4 |
| Glycerin | 1.2 |
| SFX | 1.0 |
| Poloxamer 184 | 1.0 |
| Disodium Cocoamphodiacetate | 1.0 |
| Disodium EDTA | 0.5 |
| Polyaminopropyl Biguanide | 0.2 |
| Lactic Acid | ad pH 5.4 |

Recipes 221: Micellar Water

| Ingredient | % |
|---|---|
| Aqua | ad 100 |
| Glycerin | 5.0 |
| Alcohol Denat. | 2.0 |
| Sodium Lactate | 2.0 |
| Sodium PCA | 1.0 |
| SFX | 0.5 |
| Parfum | 0.3 |
| Aloe Barbadensis Leaf Juice | 0.3 |
| Cocoyl Glutamic Acid | 0.2 |
| Sodium Cocoyl Glutamate | 0.2 |
| Glycolipids | 0.1 |
| Citric Acid | 0.1 |
| Lactic Acid | 0.1 |
| Sodium Hyaluronate | 0.1 |
| Phytosphingosine HCl | 0.1 |
| Benzyl Alcohol | 0.1 |

Recipes 222: Face Cleanser and Scrub

| Ingredient | % |
|---|---|
| Aqua | ad 100 |
| Sodium Coco-Sulfate | 4.0 |
| SFX | 2.0 |
| Glycerin | 2.0 |
| Sodium Cocoyl Glycinate | 1.5 |
| Silica | 1.5 |
| Acrylates Copolymer | 0.8 |
| PEG-7 Glyceryl Cocoate | 0.7 |
| Sodium Chloride | 0.5 |
| Polysorbate 20 | 0.5 |
| Cocamidopropyl Betaine | 0.5 |
| Panthenol | 0.2 |
| Parfum | 0.2 |
| Charcoal Powder | 0.2 |
| Salix Alba (Willow) Bark Extract | 0.2 |
| Glycol Distearate | 0.1 |
| Lecithin | 0.1 |
| Laureth-4 | 0.1 |
| Citric Acid | ad pH 5.6 |
| Lactic Acid | 0.1 |
| Tocopherol | 0.1 |
| Ascorbyl Palmitate | 0.1 |
| Disodium EDTA | 0.1 |
| Phenoxyethanol | 0.1 |
| Ethylhexylglycerin | 0.1 |
| Sodium Benzoate | 0.1 |
| Benzyl Alcohol | 0.1 |
| Potassium Sorbate | 0.1 |

### Microemulsion Recipes

Recipes 223: Microemulsion for Make-Up Removal

| Ingredient | % |
|---|---|
| Water | ad 100 |
| 1,3-Butanediol | 15.0 |
| Polyglyceryl-4 Caprate | 6.0 |
| Polyglyceryl-3 Caprate | 6.0 |
| SFX | 6.0 |
| Glycerin | 6.0 |
| Isohexadecane | 3.0 |
| Sodium Benzoate | 0.8 |
| Citric Acid | ad pH 5.0 |

Recipes 224: Microemulsion for Make-Up Removal

| Ingredient | % |
|---|---|
| Water | ad 100 |
| 1,3-Butanediol | 7.5 |
| Polyglyceryl-4 Caprate | 5.5 |
| Glycolipids | 3.0 |
| SFX | 1.5 |
| Glycerin | 5.0 |
| Sodium Benzoate | 0.8 |
| Citric Acid | Ad pH 5.0 |

### Toothpaste Recipes

Recipes 225: Toothpaste

| Ingredient | % |
|---|---|
| Aqua | ad 100 |
| Glycerin | 29.0 |
| Sodium Metaphosphate | 12.0 |
| Hydrated Silica (Hydrated) | 10.0 |
| Xylitol | 10.0 |
| SFX | 1.0 |
| Olaflur | 0.13 |
| Cocamidopropyl Betaine | 0.5 |
| Propylene Glycol | 1.0 |
| Chamomilla Recutita Flower Extract | 0.4 |
| Alcohol Denat | 0.5 |
| Panthenol | 0.1 |
| Sodium Fluoride | 0.1 |
| Hydroxyethylcellulose | 1.0 |
| Titanium Dioxide | 0.4 |
| Aroma | 1.0 |
| Citronellol | 0.0 |
| Eucalyptol | 0.2 |
| Eugenol | 0.1 |
| Menthol | 0.15 |
| Dyes, pH adjuster, preservative | q. s. |

Recipes 226: Toothpaste

| Ingredient | % |
|---|---|
| Aqua | ad 100 |
| Hydrated Silica (Hydrated) | 22.0 |
| Sorbitol | 30.0 |
| Hydroxyethylcellulose | 1.5 |
| SFX | 1.0 |
| Olaflur | 0.19 |
| Aroma | 1.0 |
| Limonene | 0.1 |
| Titanium Dioxide | 0.3 |
| Saccharin | 0.2 |
| Dyes, pH adjuster, preservative | q. s. |

Recipes 227: Toothpaste

| Ingredient | % |
|---|---|
| Sorbitol | 45.0 |
| Aqua | ad 100 |
| Hydrated Silica (Hydrated) | 18.0 |
| Sodium Lauryl Sulfate | 1.05 |
| SFX | 0.75 |
| PEG-32 | 1.0 |
| Flavor | 1.0 |
| Cellulose Gum | 0.8 |
| Cocamidopropyl Betaine | 0.35 |
| Sodium Saccharin | 0.2 |
| Sodium Fluoride | 0.23 |
| Zinc Sulfate | 0.3 |
| Mica | 0.5 |
| Dyes solution (1% FD&C Blue No. 1) | 0.02 |
| Titanium Dioxide | 0.3 |
| Eugenol | 0.2 |
| pH adjuster, preservative | q. s. |

Recipes 228: Toothpaste

| Ingredient | % |
|---|---|
| Glycerin | 35.0 |
| Hydrated Silica (Hydrated) | 22.0 |
| Aqua | ad 100 |
| Sodium Bicarbonate | 20.0 |
| PEG-12 | 3.0 |
| SFX | 1.5 |
| Sodium Lauryl Sulfate | 1.0 |
| Flavor | 1.0 |
| Sodium Hydroxide | 0.9 |
| Cellulose Gum | 0.9 |
| Chondrus Crispus | 0.5 |
| Sodium Saccharin | 0.1 |
| Calcium Peroxide | 0.1 |
| Titanium Dioxide | 0.4 |
| Sodium monofluorophosphate | 0.76 |
| PH adjuster, preservative | q. s. |

Recipes 229: Toothpaste

| Ingredient | % |
|---|---|
| Aqua | ad 100 |
| Glycerin | 33.0 |
| Hydrated Silica (Hydrated) | 24.0 |
| SFX | 1.5 |
| Cellulose Gum | 0.8 |
| Aroma | 0.9 |
| Sodium Fluoride | 0.23 |
| Sodium Saccharin | 0.2 |
| Commiphora Myrrha Oil | 0.05 |
| Salvia Officinalis Oil | 0.05 |
| Mentha Piperita Oil | 0.2 |
| Chamomilla Recutita Flower Extract | 0.2 |
| Limonene | 0.1 |
| Titanium Dioxide | 0.3 |
| Dyes solution (1% FD&C Blue No. 1) | 0.05 |
| pH adjuster, preservative | q.s. |

Recipes 230: Toothpaste

| Ingredient | % |
|---|---|
| Glycerin | 27.0 |
| Hydrated Silica (Hydrated) | 21.0 |
| Sodium Hexametaphosphate | 13.0 |
| Aqua | ad 100 |
| PEG-6 | 5.0 |
| Flavor | 1.0 |
| Trisodium Phosphate | 1.0 |
| Sodium Lauryl Sulfate | 1.05 |
| SFX | 0.35 |
| Chondrus Crispus | 0.7 |
| Cocamidopropyl Betaine | 0.1 |
| Sodium Saccharin | 0.2 |
| Polyethylene Glycol | 0.5 |
| Xanthan Gum | 0.7 |
| Sucralose | 0.5 |
| Mica | 0.3 |
| Titanium Dioxide | 0.5 |
| Sodium Fluoride | 0.32 |
| Dyes, pH adjuster, preservative | q.s. |

Recipes 231: Toothpaste

| Ingredient | % |
|---|---|
| Aqua | ad 100 |
| Sorbitol | 29.0 |
| Hydrated Silica (Hydrated) | 22.0 |
| Disodium Pyrophosphate | 5.0 |
| SFX | 1.0 |
| Glycolipids | 1.0 |
| Cellulose Gum | 1.0 |
| Aroma | 0.9 |
| Sodium Bicarbonate | 0.8 |
| Carbomer | 0.8 |
| Sodium Fluoride | 0.32 |
| Sodium Saccharin | 0.1 |
| Xanthan Gum | 0.5 |
| Titanium Dioxide | 0.5 |
| Limonene | 0.2 |
| Dyes, pH adjuster, preservative | q.s. |

Recipes 232: Toothpaste

| Ingredient | % |
|---|---|
| Aqua | ad 100 |
| Hydrated Silica (Hydrated) | 21.0 |
| Sorbitol | 19.0 |
| Glycerin | 15.0 |
| Propylene Glycol | 5.0 |
| Tetrapotassium Pyrophosphate | 5.0 |
| Pentasodium Triphosphate | 5.0 |
| Sodium C14-16 Olefin Sulfonate | 0.5 |
| Glycolipids | 0.5 |
| SFX | 0.5 |
| Disodium Pyrophosphate | 0.7 |
| Aroma | 0.3 |
| Titanium Dioxide | 0.4 |
| Xanthan Gum | 0.7 |
| Sodium Fluoride | 0.32 |
| Sodium Saccharin | 0.15 |
| Allantoin | 0.2 |
| Chamomilla Recutita Flower Extract | 0.1 |
| Salvia Officinalis Leaf Extract | 0.2 |
| Zinc Chloride | 0.3 |
| Dyes solution (1% FD&C Blue No. 1) | 0.05 |
| pH adjuster, preservative | q.s. |

Recipes 233: Toothpaste

| Ingredient | % |
|---|---|
| Aqua | ad 100 |
| Hydrated Silica (Hydrated) | 21.0 |
| Xylitol | 10.0 |
| Sorbitol | 10.0 |
| SFX | 1.5 |
| Betaine | 0.5 |
| Aloe Barbadensis Leaf Juice Powder | 0.3 |
| Xanthan Gum | 0.5 |
| Zinc Gluconate | 0.5 |
| Sodium Coco-sulfate | 0.5 |
| Mentha Spicata Crispa Herb Oil | 0.1 |
| Calendula Officinalis Flower Extract | 0.2 |
| Chamomilla Recutita Flower Extract | 0.2 |
| L-limonene | 0.1 |
| Mentha Arvensis Leaf Oil | 0.1 |
| Sodium Fluoride | 0.32 |
| Titanium Dioxide | 0.4 |
| PH adjuster, preservative | q.s. |

Recipes 234: Toothpaste

| Ingredient | % |
|---|---|
| Aqua | ad 100 |
| Hydroxyapatite | 20.0 |
| Propylene Glycol | 10.0 |
| Glycerin | 10.0 |
| Sorbitol | 10.0 |
| Tetrapotassium Pyrophosphate | 7.0 |
| Silica | 7.0 |
| SFX | 1.0 |
| Aroma | 1.0 |
| Cellulose Gum | 1.0 |
| Sodium C14-16 Olefin Sulfonate | 1.0 |
| Sodium Fluoride | 0.32 |
| Sodium Cocoyl Isethionate | 0.5 |
| Sodium Saccharin | 0.15 |
| Limonene | 0.2 |
| Titanium Dioxide | 0.4 |
| Dyes, pH adjuster, preservative | q.s. |

Recipes 235: Toothpaste

| Ingredient | % |
|---|---|
| Aqua | ad 100 |
| Hydrogenated Starch Hydrolysate | 20.0 |
| (Hydrogenated, Hydrolysed) | |
| Hydrated Silica (Hydrated), | 15.0 |
| Glycerin | 15.0 |
| Alumina | 10.0 |
| Sodium Lauryl Sulfate | 1.05 |
| PEG-12 | 1.0 |
| SFX | 0.75 |
| Perlite | 0.5 |
| Pentapotassium Triphosphate | 0.8 |
| Pentasodium Triphosphate | 0.8 |
| Sodium Monofluorophosphate | 0.76 |
| Cellulose Gum | 0.9 |
| Sodium Saccharin | 0.2 |
| Cocamidopropyl Betaine | 0.35 |
| Aroma | 1.0 |
| Limonene | 0.1 |
| Dyes solution (1% FD&C Blue No. 1) | 0.05 |
| Titanium Dioxide | 0.3 |
| pH adjuster, preservative | q.s. |

Recipes 236: Toothpaste

| Ingredient | % |
|---|---|
| Dicalcium Phosphate Dihydrate (Dihydrate) | 25.0 |
| Aqua | ad 100 |
| Sorbitol | 20.0 |
| Glycerin | 20.0 |
| Methyl Salicylate | 1.0 |
| Sodium Lauryl Sulfate | 0.9 |
| SFX | 0.9 |
| Titanium Dioxide | 0.5 |
| Chondrus Crispus | 0.8 |
| Cellulose Gum | 0.5 |
| Sodium Silicate | 0.3 |
| Hydrated Silica (Hydrated) | 1.0 |
| Thymol | 0.1 |
| Sodium Saccharin | 0.2 |
| Sodium Hydroxide | 0.5 |
| Menthol | 0.3 |
| Dyes solution (1% FD&C Blue No.1) | 0.02 |
| pH adjuster, preservative | q.s. |

Recipes 237: Toothpaste

| Ingredient | % |
|---|---|
| Aqua | ad 100 |
| Hydrated Silica (Hydrated) | 21.0 |
| Sorbitol | 21.0 |
| Glycerin | 18.0 |
| PEG-6 | 5.0 |
| SFX | 1.5 |
| Xanthan Gum | 0.8 |
| Flavor | 0.7 |
| Titanium Dioxide | 0.5 |
| Sodium Citrate | 0.4 |
| Sodium Fluoride | 0.32 |
| Zinc Chloride | 0.5 |
| Sodium Saccharin | 0.2 |
| Chondrus Crispus | 0.7 |
| Limonene | 0.1 |
| Dyes solution (1% FD&C Blue No. 1) | 0.04 |
| pH adjuster, preservative | q.s. |

Recipes 238: Toothpaste

| Ingredient | % |
|---|---|
| Aqua | ad 100 |
| Sorbitol | 36.0 |
| Hydrated Silica (Hydrated), | 18.0 |
| Glycerin | 18.0 |
| Potassium Nitrate | 1.0 |
| Aroma | 1.0 |
| Cocamidopropyl Betaine | 1.0 |
| SFX | 1.0 |
| Xanthan Gum | 0.7 |
| Sodium Saccharin | 0.2 |
| Sodium Fluoride | 0.32 |
| Mica | 0.4 |
| Titanium Dioxide | 0.4 |
| Sodium Hydroxide | 0.5 |
| Limonene | 0.1 |
| Eugenol | 0.1 |
| Dyes, pH adjuster, preservative | q.s. |

Recipes 239: Toothpaste

| Ingredient | % |
|---|---|
| Glycerin | 33.0 |
| PEG-8 | 5.0 |
| Hydrated Silica (Hydrated), | 5.0 |
| Calcium Sodium Phosphosilicate | 3.0 |
| Cocamidopropyl Betaine | 1.0 |
| SFX | 1.0 |
| Sodium Methyl Cocoyl Taurate | 0.3 |
| Aroma | 0.8 |
| Titanium Dioxide | 0.5 |
| Carbomer | 0.5 |
| Sodium Saccharin | 0.2 |
| Sodium Fluoride | 0.32 |
| Limonene | 0.2 |
| Dyes, pH adjuster, preservative | q.s. |

Recipes 240: Toothpaste

| Ingredient | % |
|---|---|
| Aqua | ad 100 |
| Sorbitol | 63.0 |
| Hydrated Silica (Hydrated) | 14.0 |
| Silica | 7.0 |
| SFXs | 1.0 |
| Titanium Dioxide | 0.6 |
| Maris Sal | 0.5 |
| Glycolipdis | 0.5 |
| Sodium Cocoyl Glutamate | 0.5 |
| Disodium Cocoyl Glutamate | 0.5 |
| Xanthan Gum | 0.7 |
| Sodium Fluoride | 0.32 |
| Echinacea Purpurea Extract (Extract) | 0.1 |
| Arnica Montana Flower Extract | 0.1 |
| Mentha Piperita Leaf Water | 0.1 |
| Myrtus Communis Leaf Water | 0.1 |
| Glycerin | 1.0 |
| Menthol | 0.2 |
| Aroma | 1.0 |
| L-limonene | 0.2 |
| Dyes, pH adjuster, preservative | q.s. |

Recipes 241: Toothpaste

| Ingredient | % |
|---|---|
| Sorbitol | 33.0 |
| Aqua | ad 100 |
| Hydrated Silica (Hydrated) | 18.0 |
| Glycerin | 15.0 |
| PEG-32 | 4.0 |
| Sodium Lauryl Sulfate | 1.5 |
| Cellulose Gum | 1.0 |
| SFX | 1.0 |
| Sodium Saccharin | 0.3 |
| Eucalyptol | 0.2 |
| Methyl Salicylate | 0.5 |
| Thymol | 0.1 |
| Phosphoric Acid | 0.3 |
| Menthol | 0.4 |
| Zinc Citrate | 0.5 |
| Sodium Phosphate | 0.2 |
| Xanthan Gum | 0.5 |
| Benzoic Acid | 0.2 |
| Sodium Monofluorophosphate | 0.76 |
| Flavor | 1.0 |
| Dyes solution (1% FD&C Blue No. 1) | 0.05 |
| pH adjuster, preservative | q.s. |

Recipes 242: Toothpaste

| Ingredient | % |
|---|---|
| Hydrogenated Starch Hydrolysate | 30.0 |
| (Hydrogenated, Hydrolysed) | |
| Aqua | ad 100 |
| Hydrated Silica (Hydrated) | 22.0 |
| PEG-32 | 4.0 |
| SFX | 1.0 |
| Aroma | 1.0 |
| Cellulose Gum | 1.0 |
| Sodium Fluoride | 0.32 |
| Sodium Saccharin | 0.25 |
| PVM/MA Copolymer | 0.7 |
| Trisodium Phosphate | 0.8 |
| Calcium Aluminum Borosilicate | 0.4 |
| Glycerin | 0.9 |
| Lecithin | 0.2 |
| Tin Oxide | 0.2 |
| Limonene | 0.1 |
| Dyes solution (1% FD&C Blue No. 1) | 0.04 |
| Titanium Dioxide | 0.3 |
| pH adjuster, preservative | q.s. |

Recipes 243: Toothpaste

| Ingredient | % |
|---|---|
| Aqua | ad 100 |
| Sorbitol | 50.0 |
| Hydrated Silica (Hydrated), | 14.0 |
| Silica Dimethyl Silylate | 3.5 |
| Hydroxyethylcellulose | 3.5 |
| SFX | 1.05 |
| PEG-40 Hydrogenated Castor | 0.8 |
| Oil (Hydrogenated) | |
| Aroma | 0.9 |
| Sodium Gluconate | 0.4 |
| Limonene | 0.1 |
| PEG-3 tallow aminopropyl amine | 0.5 |
| Olaflur | 0.06 |
| Stannous Fluoride | 0.4 |
| Sodium Saccharin | 0.2 |
| Potassium Hydroxide | 0.4 |
| Hydrochloric Acid | 0.2 |
| Dyes solution (1% FD&C Blue No. 1) | 0.04 |
| pH adjuster, preservative | q.s. |

Recipes 244: Toothpaste

| Ingredient | % |
|---|---|
| Aqua | ad 100 |
| Sorbitol | 45.0 |
| Hydrated Silica (Hydrated) | 21.0 |
| Sodium Lauryl Sulfate | 1.5 |
| SFX | xx |
| Cellulose Gum | 1.0 |
| Aroma | 1.0 |
| Zinc Citrate | 0.5 |
| Chondrus Crispus Powder (Powdered) | 0.8 |
| Sodium Saccharin | 0.2 |
| Sodium Fluoride | 0.1 |
| Titanium Dioxide | 0.5 |
| Hydroxyethylcellulose | 0.3 |
| Sodium Citrate | 0.2 |
| Stannous Fluoride | 0.4 |
| Dyes, pH adjuster, preservative | q.s. |

Recipes 245: Toothpaste

| Ingredient | % |
|---|---|
| Sodium Bicarbonate | 35.00 |
| PEG-8 | 5.0 |
| Hydrated Silica (Hydrated | 18.0 |
| Glycerin | 18.0 |
| SFX | 1.0 |
| Sodium Lauryl Sulfate | 0.5 |
| Sodium Saccharin | 0.5 |
| Aroma | 0.9 |
| Dipotassium Phosphate | 0.8 |
| Sodium Carbonate | 0.5 |
| Sodium Fluoride | 0.32 |
| PEG/PPG-116/66 Copolymer | 0.8 |
| Calcium Peroxide | 0.5 |
| Calcium Hydroxide | 0.4 |
| Dyes solution (1% FD&C Blue No.1) | 0.04 |
| Sodium Silicate | 0.5 |
| Limonene | 0.1 |
| Silica | 0.9 |
| Titanium Dioxide | 0.3 |
| Dyes, pH adjuster, preservative | q.s. |

Recipes 246: Toothpaste

| Ingredient | % |
|---|---|
| Glycerin | 33.0 |
| Hydrated Silica | 22.0 |
| Aqua | ad 100 |
| Sorbitol | 15.0 |
| Tetrapotassium Pyrophosphate | 3.5 |
| SFX | 1.5 |
| Titanium Dioxide | 0.4 |
| Sodium Lauroyl Sarcosinate | 0.5 |
| Cellulose Gum | 0.8 |
| Lauryl Glucoside | 0.5 |
| PVP | 0.5 |
| Cocamidopropyl Betaine | 0.5 |
| Xanthan Gum | 0.5 |
| Stannous Fluoride | 0.4 |
| Dyes, pH adjuster, preservative | q.s. |

Recipes 247: Toothpaste

| Ingredient | % |
|---|---|
| Aqua | ad 100 |
| Hydrated Silica (Hydrated) | 20.0 |
| Sorbitol | 18.0 |
| Xylitol | 14.0 |
| SFX | 1.2 |
| Propylene Glycol | 1.0 |
| Glycerin | 1.0 |
| Sodium C14-16 Olefin Sulfonate | 0.3 |
| Aroma | 1.0 |
| Xanthan Gum | 0.5 |
| Disodium Phosphate | 0.4 |
| Sodium Fluoride | 0.32 |
| Zinc Chloride | 0.5 |
| Sodium Saccharin | 0.2 |
| Citric Acid | 0.2 |
| Titanium Dioxide | 0.4 |
| Dyes, pH adjuster, preservative | q.s. |

Recipes 248: Toothpaste

| Ingredient | % |
|---|---|
| Aqua | ad 100 |
| Hydrogenated Starch Hydrolysate | 25.0 |
| (Hydrogenated, Hydrolysed) | |
| Hydrated Silica (Hydrated) | 21.0 |
| Hydroxyapatite | 4.5 |
| PEG-32 | 4.5 |
| SFX | 1.0 |
| Sodium Lauryl Sulfate | 0.5 |
| Aroma | 0.9 |
| Sodium Monofluorophosphate | 0.76 |
| Trisodium Phosphate | 0.5 |
| Cellulose Gum | 1.0 |
| Sodium Saccharin | 0.2 |
| Sodium Hydroxide | 0.3 |
| Limonene | 0.1 |
| Dyes solution (1% FD&C Blue No.1) | 0.05 |
| Titanium Dioxide | 0.4 |
| pH adjuster, preservative | q.s. |

Recipes 249: Toothpaste

| Ingredient | % |
|---|---|
| Glycerin | 32.0 |
| Hydrated Silica (Hydrated) | 21.0 |
| Aqua | ad 100 |
| Sodium Bicarbonate | 30.0 |
| PEG-12 | 3.0 |
| SFX | 1.5 |
| Sodium Lauryl Sulfate | 1.0 |
| Flavor | 1.0 |
| Sodium Hydroxide | 1.0 |
| Cellulose Gum | 1.0 |
| Chondrus Crispus | 0.7 |
| Sodium Saccharin | 0.15 |
| Calcium Peroxide | 0.5 |
| Titanium Dioxide | 0.5 |
| Sodium Monofluorophosphate | 0.76 |
| Dyes, pH adjuster, preservative | q.s. |

Recipes 250: Toothpaste

| Ingredient | % |
|---|---|
| Aqua | ad 100 |
| Sorbitol | 63.0 |
| Hydrated Silica (Hydrated) | 22.0 |
| Propylene Glycol | 1.5 |
| Cellulose Gum | 1.5 |
| SFX | 1.0 |
| Sodium C14-16 Olefin Sulfonate | 0.5 |
| Aroma | 0.8 |
| Calcium Glycerophosphate | 0.4 |
| Sodium Fluoride | 0.11 |
| Sodium Saccharin | 0.4 |
| Cocamidopropyl Betaine | 0.5 |
| Citral | 0.1 |
| Geraniol | 0.1 |
| Limonene | 0.1 |
| Linalool | 0.1 |
| Titanium Dioxide | 0.2 |
| Dyes, pH adjuster, preservative | q.s. |

Recipes 251: Toothpaste

| Ingredient | % |
|---|---|
| Aqua | ad 100 |
| Calcium Carbonate | 30.0 |
| Silica | 7.0 |
| Glycerin | 5.0 |
| Hydrated Silica | 2.5 |
| SFX | 1.5 |
| Aroma | 1.0 |
| Chondrus Crispus Powder | 0.5 |
| Cellulose Gum | 0.5 |
| Chlorhexidine Gluconate | 0.1 |
| Limonene | 0.15 |
| Sodium Saccharin | 0.15 |
| Titanium Dioxide | 0.1 |
| Sodium Fluoride | 0.32 |
| Dyes, pH adjuster, preservative | q.s. |

Recipes 252: Toothpaste

| Ingredient | % |
|---|---|
| Aqua | ad 100 |
| Hydrogenated Starch Hydrolysate | 20.0 |
| Hydrated Silica | 15.0 |
| PEG-32 | 4.0 |
| Glycolipids | 1.5 |
| SFX | 1.0 |
| Propylene Glycol | 0.9 |
| Aroma | 0.9 |
| Sodium Lauryl Sulfat | 0.5 |
| Hydroxyethylcellulose | 0.5 |
| Olaflur | 0.13 |
| Titanium Dioxide | 0.1 |
| Sodium Fluoride | 0.1 |
| Sodium Saccharin | 0.2 |
| Chamomilla Recutita Flower Extract | 0.2 |
| Chlorhexidine Digluconate | 0.1 |
| Limonene | 0.1 |
| Citral | 0.1 |
| Dyes, pH adjuster, preservative | q.s. |

Recipes 253: Toothpaste

| Ingredient | % |
|---|---|
| Aqua | ad 100 |
| Sorbitol | 20.0 |
| Hydrated Silica | 15.0 |
| Silica | 2.0 |
| SFX | 1.0 |
| Cocamidopropyl Betaine | 0.75 |
| Titanium Dioxide | 0.15 |
| Flavor | 0.8 |
| Linalool | 0.2 |
| Sodium Monofluorophosphate | 0.76 |
| Sodium Fluoride | 0.1 |
| Sodium Saccharin | 0.25 |
| Cellulose Gum | 0.5 |
| Limonene | 0.1 |
| Chlorhexidine Digluconate | 0.12 |
| Dyes, pH adjuster, preservative | q.s. |

Recipes 254: Toothpaste

| Ingredient | % |
|---|---|
| Aqua | ad 100 |
| Hydrated Silica | 15.0 |
| Glycerin | 15.0 |
| Potassium Nitrate | 5.0 |
| Sorbitol | 4.5 |
| Sodium Cocoamphoacetate | 0.5 |
| SFX | 0.5 |
| PEG-40 Hydrogenated Castor Oil | 0.5 |
| Carboxymethyl Cellulose | 0.5 |
| Titanium Dioxide | 0.1 |
| Sodium Fluoride | 0.32 |
| Sodium Saccharin | 0.2 |
| Aroma | 0.2 |
| Chlorhexidine Digluconate | 0.12 |
| Dyes, pH adjuster, preservative | q.s. |

Recipes 255: Toothpaste for Kids

| Ingredient | % |
|---|---|
| Aqua | ad 100 |
| Sorbitol | 28.0 |
| Hydrated Silica (Hydrated) | 18.0 |
| Hydroxyethylcellulose | 1.5 |
| Titanium Dioxide | 0.5 |
| Cocamidopropyl Betaine | 0.5 |
| SFX | 0.5 |
| Olaflur | 0.07 |
| Aroma | 0.8 |
| Limonene | 0.1 |
| Sodium Saccharin | 0.2 |
| Hydrochloric Acid | 0.7 |
| Dyes, pH adjuster, preservative | q.s. |

Recipes 256: Toothpaste for Children

| Ingredient | % |
|---|---|
| Sorbitol | 33.0 |
| Aqua | ad 100 |
| Hydrated Silica (Hydrated) | 22.0 |
| SFX | 1.0 |
| Sodium C14-16 Olefin Sulfonate | 1.0 |
| Aroma | 0.8 |
| Cellulose Gum | 0.8 |
| Calcium Glycerophosphate | 0.5 |
| Olaflur | 0.07 |
| Sodium Fluoride | 0.1 |
| Propylene Glycol | 0.5 |
| Sodium Saccharin | 0.2 |
| Cocamidopropyl Betaine | 0.35 |
| Mica | 0.5 |
| Citral | 0.1 |
| Limonene | 0.1 |
| Linalool | 0.1 |
| Titanium Dioxide | 0.4 |
| Dyes solution (1% FD&C Blue No. 1) | 0.05 |
| Olaflur | 0.07 |
| pH adjuster, preservative | q.s. |

Recipes 257: Baby Toothpaste

| Ingredient | % |
|---|---|
| Aqua | ad 100 |
| Hydrogenated Starch Hydrolysate | 14.0 |
| (Hydrogenated, Hydrolysed) | |
| Hydrated Silica (Hydrated) | 14.0 |
| PEG-32 | 3.5 |
| Silica | 7.0 |
| SFX | 1.5 |
| Cellulose Gum | 1.0 |
| Aroma | 1.0 |
| Propylene Glycol | 0.7 |
| PEG-40 Hydrogenated Castor | 0.8 |
| Oil (Hydrogenated), | |
| Olaflur | 0.06 |
| Sodium Chloride | 0.2 |
| Sodium Saccharin | 0.15 |
| Tocopheryl Acetate | 0.1 |
| Glycerin | 1.0 |
| Retinyl Palmitate | 0.1 |
| Dyes solution (1% FD&C Blue No.1) | 0.02 |
| pH adjuster, preservative | q.s. |

Recipes 258: Toothpaste for Children

| Ingredient | % |
|---|---|
| Aqua | ad 100 |
| Hydrated Silica (Hydrated) | 22.0 |
| Glycerin | 20.0 |
| Xylitol | 10.0 |
| SFX | 1.5 |
| Propylene Glycol | 0.7 |
| Xanthan Gum | 0.5 |
| Titanium Dioxide | 0.5 |
| Flavor | 0.9 |
| Sodium Lauroyl Sarcosinate | 0.5 |
| Disodium EDTA | 0.3 |
| Sodium Monofluorophosphate | 0.76 |
| Sodium Chloride | 0.2 |
| Dyes, pH adjuster, preservative | q.s. |

Recipes 259: Kids Toothpaste

| Ingredient | % |
|---|---|
| Aqua | ad 100 |
| Sorbitol | 40.0 |
| Hydrated Silica (Hydrated) | 17.0 |
| Glycerin | 26.0 |
| SFX | 1.5 |
| Xanthan Gum | 1.0 |
| Steareth-30 | 1.0 |
| Chondrus Crispus | 1.0 |
| Aroma | 1.0 |
| Disodium Phosphate | 0.5 |
| Sodium Benzoate | 0.2 |
| Amyloglucosidase | 0.1 |
| Citric Acid | 0.3 |
| Zinc Gluconate | 0.5 |
| Glucose Oxidase | 0.1 |
| Sodium Fluoride | 0.22 |
| Sodium Saccharin | 0.15 |
| Potassium Thiocyanate | 0.1 |
| Lactoperoxidase | 0.1 |
| Titanium Dioxide | 0.3 |
| Dyes, pH adjuster, preservative | q.s. |

Recipes 260: 2 in 1 Toothpaste + Mouthwash

| Ingredient | % |
|---|---|
| Sorbitol | 38.0 |
| Aqua | ad 100 |
| Hydrated Silica (Hydrated) | 21.0 |
| Pentasodium Triphosphate | 5.0 |
| Sodium Lauryl Sulfate | 0.35 |
| SFX | 1.05 |
| PEG-32 | 1.0 |
| Aroma | 1.0 |
| Alumina | 0.5 |
| Alcohols | 0.7 |
| Xanthan Gum | 0.8 |
| Sodium Fluoride | 0.23 |
| Sodium Saccharin | 0.2 |
| Disodium Phosphate | 0.5 |
| Cocamidopropyl Betaine | 0.35 |
| Zinc Sulfate | 0.5 |
| Trisodium Phosphate | 0.3 |
| Sodium Chloride | 0.4 |
| Sodium Sulfate | 0.2 |
| Limonene | 0.1 |
| Titanium Dioxide | 0.5 |
| Dyes, pH adjuster, preservative | q.s. |

Recipes 261: 2 in 1 Toothpaste + Mouthwash

| Ingredient | % |
|---|---|
| Sorbitol | 29.0 |
| Aqua | ad 100 |
| Hydrated Silica | 15.0 |
| PEG-32 | 3.0 |
| Sodium Lauryl Sulfate | 0.5 |
| SFX | 0.5 |
| Glycolipids | 0.5 |
| Aroma | 0.9 |
| Alcohols | 0.7 |
| Xanthan Gum | 0.5 |
| PEG-30 Glyceryl Stearate | 0.5 |
| Sodium Fluoride | 0.05 |
| Sodium Saccharin | 0.7 |
| Disodium Phosphate | 0.3 |
| Cocamidopropyl Betaine | 0.5 |
| Zinc Sulfate | 0.3 |
| Trisodium Phosphate | 0.5 |
| Sodium Chloride | 0.1 |
| Sodium Sulfate | 0.2 |
| Limonene | 0.02 |
| Dyes, pH adjuster, Preservative | q.s. |

### Mouthwash Recipes

Recipes 262: Mouthrinse without Alcohol

| Ingredient | % |
|---|---|
| Aqua | ad 100 |
| Glycerin | 11.0 |
| Propylene Glycol | 7.0 |
| SFX | 1.0 |
| Poloxamer 407 | 0.75 |
| Aroma | 0.9 |
| Cetylpyridinium Chloride | 0.04 |
| Sodium Fluoride | 0.05 |
| Sodium Saccharin | 0.07 |
| Menthol | 0.1 |
| Dyes, pH adjuster, Preservative | q.s. |

Recipes 263: Mouthrinse without Alcohol

| Ingredient | % |
|---|---|
| Aqua | ad 100 |
| Sorbitol | 15.0 |
| Glycerin | 7.5 |
| SFX | 1.0 |
| Xylitol | 1.0 |
| Cellulose Gum | 0.5 |
| Zinc PCA | 0.5 |
| Zinc Hydroxyapatite | 0.05 |
| Aroma | 0.9 |
| Silica | 0.05 |
| Ricinus Communis Seed Oil | 0.1 |
| Ammonium Acryloyldimethyltaurate/VP Copolymer | 0.05 |
| Mentha Arvensis Leaf Oil | 0.01 |
| Sodium Myristoyl Sarcosinate | 0.2 |
| Sodium Methyl Cocoyl Taurate | 0.2 |
| Sodium Saccharin | 0.5 |
| Sodium Fluoride | 0.05 |
| Limonene | 0.1 |
| Dyes, pH adjuster, Preservative | q.s. |

Recipes 264: Mouthrinse without Alcohol

| Ingredient | % |
|---|---|
| Aqua | ad 100 |
| Sorbitol | 20.0 |
| PEG-40 | 0.5 |
| SFX | 0.5 |
| Flavor | 0.05 |
| Xanthan Gum | 0.1 |
| Zinc Sulfate | 0.15 |
| Sodium Saccharin | 0.05 |
| Sodium Fluoride | 0.05 |
| Gellan Gum | 0.2 |
| Calcium Aluminum Borosilicate | 0.1 |
| Silica | 0.05 |
| Titanium Dioxide | 0.05 |
| Tin Oxide | 0.07 |
| Eugenol | 0.15 |
| Limonene | 0.05 |
| Linalool | 0.05 |
| Dyes, pH adjuster, Preservative | q.s. |

Recipes 265: Mouthrinse without Alcohol

| Ingredient | % |
|---|---|
| Aqua | ad 100 |
| Glycerin | 7.5 |
| SFX | 0.5 |
| Glycolipids | 0.5 |
| Flavor | 0.3 |
| Cetylpyridinium Chloride | 0.04 |
| Sodium Saccharin | 0.05 |
| Sucralose | 0.05 |
| Sodium Fluoride | 0.02 |
| Disodium Phosphate | 0.25 |
| Dyes, pH adjuster, Preservative | q.s. |

Recipes 266: Mouthrinse

| Ingredient | % |
|---|---|
| Aqua | ad 100 |
| Glycerin | 10.0 |
| Sorbitol | 10.0 |
| Cocamidopropyl Betaine | 1.0 |
| SFX | 0.5 |
| Potassium Chloride | 0.1 |
| Propylene Glycol | 0.5 |
| Aroma | 0.25 |
| Sodium Fluoride | 0.02 |
| Olaflur | 0.16 |
| Potassium Acesulfame | 0.1 |
| Sodium Chloride | 0.1 |
| Limonene | 0.03 |
| Sodium Sulfate | 0.05 |
| Dyes, pH adjuster, Preservative | q.s. |

Example Recipes 267: Mouthrinse without Alcohol

| Ingredient | % |
|---|---|
| Aqua | ad 100 |
| Sorbitol | 15.0 |
| SFX | 1.0 |
| Aloe Barbadensis Leaf Extract | 0.15 |
| Calendula Officinalis Flower Extract | 0.2 |
| Chamomilla Recutita Flower Extract | 0.2 |
| Sodium Fluoride | 0.02 |
| Calcium Glycerophosphate | 0.05 |
| Zinc Gluconate | 0.8 |
| Mentha Spicata Crispa Herb Oil | 0.05 |
| Mentha Arvensis Leaf Oil | 0.05 |
| Menthol | 0.04 |
| Dyes, pH adjuster, Preservative | q.s. |

Recipes 268: Mouthrinse without Alcohol

| Ingredient | % |
|---|---|
| Aqua | ad 100 |
| Glycerin | 15.0 |
| Sodium Gluconate | 1.5 |
| SFX | 0.5 |
| PEG-40 Hydrogenated Castor Oil | 0.5 |
| Olaflur | 0.16 |
| Aroma | 0.25 |
| Stannous Chloride | 0.05 |
| Sodium Fluoride | 0.02 |
| Cocamidopropyl Betaine | 0.35 |
| Sodium Saccharin | 0.07 |
| Dyes, pH adjuster, Preservative | q.s. |

Recipes 269: Mouthrinse without Alcohol

| Ingredient | % |
|---|---|
| Aqua | ad 100 |
| Glycerin | 10.0 |
| Sorbitol | 8.0 |
| SFX | 1.0 |
| Glycolipids | 0.5 |
| Tetrasodium Pyrophosphate | 0.25 |
| Aroma | 0.04 |
| Limonene | 0.1 |
| Sodium Fluoride | 0.02 |
| Sodium Saccharin | 0.08 |
| Dyes, pH adjuster, Preservative | q.s. |

Recipes 270: Mouthrinse without Alcohol

| Ingredient | % |
|---|---|
| Aqua | ad 100 |
| Glycerin | 7.5 |
| PEG-60 Hydrogenated Castor Oil | 1.0 |
| SFX | 0.5 |
| Sodium Citrate | 0.45 |
| Aroma | 0.3 |
| Zinc Chloride | 0.15 |
| Cetylpyridinium Chloride | 0.05 |
| Sodium Saccharin | 0.08 |
| Sodium Fluoride | 0.02 |
| Dyes, pH adjuster, Preservative | q.s. |

Recipes 271: Mouthrinse without Alcohol

| Ingredient | % |
|---|---|
| Aqua | ad 100 |
| Glycerin | 11.0 |
| SFX | 2.0 |
| Flavor | 1.0 |
| Cetylpyridinium Chloride | 0.5 |
| Poloxamer 407 | 0.1 |
| Sodium Saccharin | 0.1 |
| Sodium Fluoride | 0.02 |
| Sucralose | 0.05 |
| Mentha Viridis Leaf Oil | 0.1 |
| Dyes, pH adjuster, Preservative | q.s. |

Recipes 272: Mouthrinse without Alcohol

| Ingredient | % |
|---|---|
| Aqua | ad 100 |
| Glycerin | 12.0 |
| Sorbitol | 8.0 |
| Potassium Nitrate | 0.15 |
| PEG-60 Hydrogenated Castor Oil | 0.5 |
| SFX | 0.25 |
| Poloxamer 407 | 0.25 |
| Aroma | 0.2 |
| Disodium Phosphate, Sodium Phosphate | 0.25 |
| Sodium Fluoride | 0.05 |
| Sodium Saccharin | 0.08 |
| Dyes, pH adjuster, Preservative | q.s. |

Recipes 273: Mouthrinse without Alcohol

| Ingredient | % |
|---|---|
| Aqua | ad 100 |
| Sorbitol | 10.0 |
| PEG-40 Hydrogenated Castor Oil | 1.0 |
| Potassium Citrate | 1.0 |
| SFX | 0.25 |
| Glycerin | 0.9 |
| Aroma | 0.4 |
| Zinc Sulfate | 0.15 |
| Sodium Saccharin | 0.1 |
| Propylene Glycol | 0.3 |
| Citrus Limon Juice | 0.05 |
| Aloe Barbadensis Leaf Extract | 0.1 |
| Limonene | 0.05 |
| Sodium Fluoride | 0.02 |
| Dyes, pH adjuster, Preservative | q.s. |

Recipes 274: Mouthrinse without Alcohol

| Ingredient | % |
|---|---|
| Aqua | ad 100 |
| Glycerin | 12.0 |
| SFX | 1.2 |
| Gycolipids | 1.0 |
| Hydrogenated Starch Hydrolysate | 1.0 |
| Xylitol | 1.0 |
| Steareth-30 | 0.2 |
| Amyloglucosidase | 0.5 |
| Allantoin | 0.1 |
| Glucose Oxidase | 0.5 |
| Sodium Fluoride | 0.05 |
| Limonene | 0.1 |
| Dyes, pH adjuster, Preservative | q. s. |

Recipes 275: Mouthrinse

| Ingredient | % |
|---|---|
| Aqua | ad 100 |
| Glycerin | 11.0 |
| Propylene Glycol | 7.5 |
| Sorbitol | 7.5 |
| Tetrapotassium Pyrophosphate | 1.5 |
| Polysorbate 20 | 0.5 |
| SFX | 0.5 |
| Tetrasodium Pyrophosphate | 0.75 |
| Zinc Citrate | 0.15 |
| PVM/MA Copolymer | 0.1 |
| Flavor | 0.2 |
| Sodium Fluoride | 0.05 |
| Sodium Saccharin | 0.07 |
| Dyes, pH adjuster, Preservative | q. s. |

Recipes 276: Mouthrinse

| Ingredient | % |
|---|---|
| Aqua | ad 100 |
| Glycerin | 11.0 |
| Alcohol | 7.5 |
| Sorbitol | 7.5 |
| SFX | 1.0 |
| Poloxamer 407 | 0.5 |
| Sodium Phosphate, Disodium Phosphate | 0.15 |
| Flavor | 0.9 |
| Cetylpyridinium Chloride | 0.05 |
| Sodium Saccharin | 0.08 |
| Sodium Fluoride | 0.05 |
| Dyes, pH adjuster, Preservative | q. s. |

Recipes 277: Mouthrinse

| Ingredient | % |
|---|---|
| Aqua | ad 100 |
| Alcohols | 22.0 |
| Sorbitol | 10.0 |
| Flavor | 1.0 |
| SFX | 1.0 |
| Sodium Saccharin | 0.09 |
| Eucalyptol | 0.1 |
| Thymol | 0.06 |
| Sucralose | 0.06 |
| Sodium Fluoride | 0.05 |
| Menthol | 0.04 |
| Dyes, pH adjuster, Preservative | q. s. |

Recipes 278: Mouthrinse

| Ingredient | % |
|---|---|
| Aqua | ad 100 |
| Glycerin | 12.0 |
| Xylitol | 7.0 |
| Disodium Phosphate | 1.5 |
| SFX | 1.0 |
| Cocamidopropyl Betaine | 0.5 |
| Propylene Glycol | 0.5 |
| Stannous Fluoride | 0.09 |
| Aroma | 0.3 |
| Mentha Piperita Leaf Extract | 0.1 |
| Chamomilla Recutita Flower Extract | 0.2 |
| Salvia Officinalis Leaf Extract | 0.25 |
| Commiphora Myrrha Resin Extract | 0.15 |
| Sodium Saccharin | 0.1 |
| Citral | 0.05 |
| Limonene | 0.02 |
| Linalool | 0.02 |
| Dyes, pH adjuster, Preservative | q. s. |

Recipes 279: Mouthrinse

| Ingredient | % |
|---|---|
| Aqua | ad 100 |
| Glycerin | 11.0 |
| Sorbitol | 11.0 |
| Propylene Glycol | 3.0 |
| Disodium Pyrophosphate | 1.5 |
| PEG-40 Hydrogenated Castor Oil | 1.0 |
| SFX | 0.5 |
| PVM/MA Copolymer | 0.5 |
| Tetrapotassium Pyrophosphate | 0.25 |
| Sodium Levulinate | 0.25 |
| Sodium Anisate | 0.25 |
| Olaflur | 0.16 |
| Aroma | 0.8 |
| Arginine | 0.8 |
| Sodium Saccharin | 0.07 |
| Sodium Fluoride | 0.02 |
| Dyes, pH adjuster, Preservative | q. s. |

Recipes 280: Mouthrinse

| Ingredient | % |
|---|---|
| Aqua | ad 100 |
| Alcohol Denat. | 33.0 |
| Glycerin | 10.0 |
| Sorbitol | 8.0 |
| PEG-40 Hydrogenated Castor Oil | 1.0 |
| SFX | 1.0 |
| Glycolipids | 0.5 |
| Aroma | 0.6 |
| Menthol | 0.05 |
| Sodium Saccharin | 0.07 |
| Aluminum Lactate | 0.2 |
| Thymol | 0.06 |
| Curcuma Xanthorrhiza Root Extract | 0.05 |
| Sodium Fluoride | 0.02 |
| Dyes, pH adjuster, Preservative | q. s. |

Recipes 281: Mouthrinse

| Ingredient | % |
|---|---|
| Aqua | ad 100 |
| Sorbitol | 12.5 |
| Propylene Glycol | 3.5 |
| SFX | 0.5 |
| Sodium Lauryl Sulfate | 0.5 |
| Poloxamer 407 | 0.5 |
| Flavor | 0.3 |
| Eucalyptol | 0.08 |
| Methyl Salicylate | 0.1 |
| Thymol | 0.05 |
| Stannous Fluoride | 0.09 |
| Menthol | 0.1 |
| Sodium Saccharin | 0.06 |
| Sucralose | 0.7 |
| Camellia Sinensis Leaf Extract | 0.05 |
| Dyes, pH adjuster, Preservative | q. s. |

Recipes 282: Mouthrinse

| Ingredient | % |
|---|---|
| Aqua | ad 100 |
| SFX | 1.0 |
| Eucalyptol | 0.9 |
| Zinc Chloride | 0.25 |
| Methyl Salicylate | 0.05 |
| Thymol | 0.05 |
| Levomenthol | 0.07 |
| Stannous Fluoride | 0.09 |
| Dyes, pH adjuster, Preservative | q. s. |

Recipes 283: Mouthrinse

| Ingredient | % |
|---|---|
| Aqua | ad 100 |
| Alcohol Denat. | 15.0 |
| Sorbitol | 5.0 |
| Glycine | 1.0 |
| SFX | 1.0 |
| Aroma | 0.2 |
| Zinc Sulfate | 0.2 |
| Sodium Fluoride | 0.02 |
| Sodium Saccharin | 0.07 |
| Dyes, pH adjuster, Preservative | q. s. |

Recipes 284: Mouthrinse

| Ingredient | % |
|---|---|
| Aqua | ad 100 |
| Sorbitol | 11.0 |
| Alcohol Denat. | 11.0 |
| Xylitol | 5.0 |
| PEG-40 Hydrogenated Castor Oil | 0.75 |
| SFX | 0.5 |
| Isopropyl Alcohol | 0.5 |
| Menthol | 0.05 |
| Sodium Citrate | 0.15 |
| Mentha Piperita Herb Oil | 0.25 |
| Sodium Saccharin | 0.08 |
| Methyl Diisopropyl Propionamide | 0.05 |
| Thymol | 0.07 |
| Glycolipids | 0.1 |
| Sodium Fluoride | 0.05 |
| O-cymen-5-ol | 0.05 |
| Cyclodextrin | 0.07 |
| Eugenia Caryophyllus Leaf Oil | 0.1 |
| Propylene Glycol | 0.25 |
| Butylene Glycol | 0.25 |
| Psidium Guajava Leaf Extract | 0.08 |
| Phyllanthus Emblica Fruit Extract | 0.08 |
| Echinacea Purpurea Root Extract | 0.08 |
| Salvia Officinalis Leaf Extract | 0.2 |
| Dyes, pH adjuster, Preservative | q. s. |

Recipes 285: Mouthrinse

| Ingredient | % |
|---|---|
| Aqua | ad 100 |
| Alcohols | 15.0 |
| Sorbitol | 7.0 |
| Sodium Lauroyl Sarcosinate | 1.0 |
| SFX | 0.25 |
| PEG-40 Hydrogenated Castor Oil | 0.25 |
| Aroma | 0.4 |
| Cetylpyridinium Chloride | 0.04 |
| Sodium Citrate | 0.15 |
| Sodium Saccharin | 0.08 |
| Sodium Fluoride | 0.05 |
| Limonene | 0.05 |
| Dyes, pH adjuster, Preservative | q. s. |

Recipes 286: Mouthrinse

| Ingredient | % |
|---|---|
| Aqua | ad 100 |
| Alcohol denat. | 18.0 |
| Sorbitol | 8.0 |
| Aroma | 1.0 |
| Poloxamer 407 | 0.9 |
| Methyl salicylate | 0.2 |
| Sodium Saccharin | 0.2 |
| Sodium Fluoride | 0.05 |
| SFX | 0.9 |
| Chlorhexidine Digluconate | 0.2 |
| PEG-40 Hydrogenated Castor Oil | 0.2 |
| Dyes, pH adjuster, Preservative | q. s. |

Recipes 287: Mouthrinse

| Ingredient | % |
|---|---|
| Aqua | ad 100 |
| Glycerin | 10.0 |
| Hydrogenated Starch Hydrolysate | 1.0 |
| PVP | 0.5 |
| SFX | 0.5 |
| Aroma | 0.4 |
| Zinc Acetate | 0.2 |
| Sodium Fluoride | 0.32 |
| Potassium Acesulfame | 0.15 |
| Chlorhexidine Digluconate | 0.2 |
| Dyes, pH adjuster, Preservative | q. s. |

Recipes 288: Mouthrinse

| Ingredient | % |
|---|---|
| Aqua | ad 100 |
| Glycerin | 12.0 |
| Sorbitol | 7.0 |
| SFX | 0.75 |
| PEG-40 Hydrogenated Castor Oil | 0.5 |
| Chlorhexidine Gluconate | 0.2 |
| Mentha Piperita Oil | 0.2 |
| Sodium Fluoride | 0.05 |
| Sodium Saccharin | 0.2 |
| Dyes, pH adjuster, Preservative | q. s. |

Recipes 289: Mouthrinse

| Ingredient | % |
|---|---|
| Aqua | ad 100 |
| Glycerin | 15.0 |
| Macrogolglycerol Hydroxystearate | 7.0 |
| Sorbitol | 7.0 |
| SFX | 0.5 |
| Cocamidopropyl Betaine | 0.2 |
| Sodium Fluoride | 0.02 |
| Chlorhexidine Digluconate | 0.2 |
| Flavor | 0.5 |
| Dyes, pH adjuster, Preservative | q.s. |

Recipes 290: Mouthrinse Concentrate

| Ingredient | % |
|---|---|
| Propylene Glycol | 55.0 |
| Aqua | ad 100 |
| Aroma | 2.0 |
| Sodium Saccharin | 0.4 |
| SFX | 0.75 |
| Polysorbate 20 | 0.25 |
| Commiphora Myrrha Oil | 2.0 |
| Salvia Officinalis Oil | 0.2 |
| Mentha Piperita Oil | 0.2 |
| Chamomilla Recutita Flower Extract | 0.2 |
| Limonene | 0.1 |
| Sodium Fluoride | 0.02 |
| Dyes, pH adjuster, Preservative | q.s. |

Recipes 291: Mouthrinse Concentrate

| Ingredient | % |
|---|---|
| Propylene Glycol | 45.0 |
| PEG-6 | 5.0 |
| Aqua | ad 100 |
| SFX | 1.0 |
| Bisabolol | 0.2 |
| Zinc Chloride | 0.25 |
| Cetylpyridinium Chloride | 0.1 |
| Sodium Saccharin | 0.5 |
| Sodium Fluoride | 0.05 |
| Salvia Officinalis Oil | 2.5 |
| Eugenol | 0.2 |
| Limonene | 0.1 |
| Linalool | 0.1 |
| Citronellol | 0.1 |
| Dyes, pH adjuster, Preservative | q.s. |

Recipes 292: Mouthrinse Concentrate

| Ingredient | % |
|---|---|
| Alcohols | 55.0 |
| Aqua | ad 100 |
| Aroma | 1.5 |
| SFX | 0.7 |
| Sodium C14-17 Alkyl Sulfonate | 0.7 |
| Mentha Arvensis Leaf Oil | 2.0 |
| Sodium Saccharin | 0.5 |
| Chamomilla Recutita Flower Extract | 0.5 |
| Commiphora Myrrha Resin Extract | 0.2 |
| Salvia Officinalis Leaf Extract | 0.2 |
| Sodium Fluoride | 0.02 |
| Limonene | 0.1 |
| Dyes, pH adjuster, Preservative | q.s. |

Recipes 293: Peroxide Mouthrinse

| Ingredient | % |
|---|---|
| Aqua | ad 100 |
| Glycerin | 11.0 |
| Alcohol | 5.0 |
| H₂O₂ 35% | 4.30 |
| Polyphosphate | 1.0 |
| Menthol | 0.05 |
| Coolant | 0.02 |
| Flavor | 0.15 |
| Calcium Chloride | 0.03 |
| SFX | 0.50 |
| Sodium Saccharin | 0.1 |
| Sodium Fluoride | 0.22 |
| Dyes, pH adjuster, Preservative | q.s. |

Recipes 294: Fluoride Gel

| Ingredient | % |
|---|---|
| Aqua | ad 100 |
| Propylene Glycol | 30.0 |
| Hydroxyethylcellulose | 3.0 |
| Saccharin | 1.0 |
| SFX | 0.5 |
| Pyrus Malus Fruit | 0.2 |
| Mentha Piperita Oil | 0.2 |
| Flavor | 0.5 |
| Aminofluoride Dectafluor | 0.29 |
| Olaflur | 3.03 |
| Sodium Fluoride | 2.21 |
| Dyes, pH adjuster, Preservative | q.s. |

Recipes 295: Fluoride Gel

| Ingredient | % |
|---|---|
| Aqua | ad 100 |
| Sorbitol | 15.00 |
| Silica | 5.0 |
| PEG 600 | 3.5 |
| Tetrapotassium Pyrophosphate | 1.5 |
| Xanthan Gum | 0.5 |
| Sodium Lauryl Sulfate | 0.75 |
| SFX | 0.75 |
| Aroma | 0.9 |
| Methol | 0.05 |
| Anethol | 0.05 |
| Citronellol | 0.2 |
| Sodium Fluoride | 5.0 |
| Dyes, pH adjuster, Preservative | q.s. |

Recipes 296: Dental Floss

| Ingredient | % |
|---|---|
| PTFE | ad 100 |
| Aroma | 1.0 |
| Acacia Senegal Gum | 0.7 |
| Cera Alba | 0.7 |
| Potassium Acesulfame | 0.5 |
| Sodium Lauryl Sulfate | 0.25 |
| SFX | 0.25 |
| Glycerin | 0.7 |
| Silica | 0.5 |
| Sodium Fluoride | 0.22 |

Recipes 297: Teeth Wipe

| Ingredient | % |
|---|---|
| Aqua | q. s. 100 |
| Alcohol Denat. | 30.00 |
| Glycerin | 20.00 |
| Stevia | 1.0 |
| Polysorbate 20 | 0.5 |
| SFX | 0.5 |
| Aroma | 1.0 |
| Dyes, pH adjuster, Preservatives | q.s. |

Recipes 298: Chewing Gum

| Ingredient | % |
|---|---|
| Gum Base | ad 100 |
| Sorbit | 40.0 |
| Isomalt | 9.0 |
| Xylitol | 2.5 |
| Mannit D | 2.5 |
| Aspartame | 0.1 |
| Acesulfam K | 0.05 |
| Emulgum | 0.2 |
| Sorbitol | 12.00 |
| Glycerin | 1.0 |
| SFX | 1.0 |
| Flavor | 1.0 |
| Sodium Fluoride | 0.05 |
| Dyes, pH adjuster, Preservative | q.s. |

Recipes 299: Mouthwash Tabs

| Ingredient | % |
|---|---|
| Dicalcium Phosphate Anhydrous | ad 100 |
| Sodium Bicarbonate | 35.0 |
| Silica | 7.0 |
| Citric Acid | 4.0 |
| SFX | 3.0 |
| Sorbitol | 1.0 |
| Glycerin | 1.0 |
| Stevia Rebaudiana Extract (Stevia) | 0.2 |
| Xylitol | 0.5 |
| Aroma | 0.1 |
| Mentha Piperita Oil | 0.1 |
| Dyes, pH adjuster, Preservative | q.s. |

Recipes 300: Tooth Tab

| Ingredient | % |
|---|---|
| Dicalcium Phosphate Anhydrous | ad 100 |
| Potassium Bitartrate | 15.0 |
| Sorbitol | 15.0 |
| SFX | 6.0 |
| Lauroyl Sarcosine | 0.8 |
| Kaolin | 0.5 |
| Sodium Saccharin | 0.5 |
| Aroma | 0.2 |
| Dyes, pH adjuster, Preservative | q.s. |

Recipes 301: Powder Toothpaste

| Ingredient | % |
|---|---|
| Calcium Carbonate | ad 100 |
| Sorbitol | 15.0 |
| Kaolin | 15.0 |
| Sodium Bicarbonate | 15.0 |
| SFX | 1.5 |
| Aroma | 1.0 |
| Citrus Limon Peel Oil | 1.0 |
| Stevia Rebaudiana Extract | 0.5 |
| Xylitol | 0.5 |
| Citral | 0.1 |
| Geraniol | 0.1 |
| Limonene | 0.1 |
| Dyes, pH adjuster, Preservative | q.s. |

Further Recipess (in wt.-%):

| | I | II | III | IV | V | VI | VII | VIII | IX | X |
|---|---|---|---|---|---|---|---|---|---|---|
| Water | ad 100 | | | | | | | | | |
| SFX | 9.0 | 15.0 | 5.5 | 5.0 | 7.0 | 3.0 | 6.0 | 4.0 | 3.5 | 3.0 |
| Sodium Laureth Sulfate | — | — | — | 7.0 | — | — | — | 4.5 | — | — |
| Sodium Lauryl Sulfate | — | — | — | — | 5.0 | — | — | — | 3.5 | 1.0 |
| Cocamidopropyl Betaine | — | — | — | — | — | 6.0 | — | 3.0 | 2.0 | 4.5 |
| Sodium Cocoamphoacetate | — | — | — | — | — | — | 2.0 | — | 3.5 | — |
| Coco-Glucoside | — | — | — | — | — | — | 5.5 | 1.5 | — | — |
| Sodium Cocoyl Glutamate | — | — | — | — | — | — | 0.7 | — | — | — |
| Stearic Acid | — | — | — | — | — | — | — | 1.0 | — | 3.5 |
| Glyceryl Glucoside | — | 0.5 | — | — | 0.3 | — | 0.2 | — | — | — |
| Sucrose Cocoate | 0.5 | — | 1.0 | 1.0 | 1.0 | 0.5 | — | 1.0 | 1.0 | 1.0 |
| Glycerin | — | 1.0 | 0.5 | — | — | 0.3 | 1.5 | 1.0 | — | 1.0 |
| PEG-7 Glyceryl Cocoate | — | 0.7 | — | 0.5 | — | — | — | — | — | 0.5 |
| Trideceth-9 | — | — | — | 0.2 | — | — | — | 0.3 | — | — |
| Polyglyceryl-4 Caprate | — | — | 1.0 | — | 0.4 | — | 0.5 | — | — | — |
| Polyquaternium-10 | — | 0.2 | — | 0.1 | — | — | — | 0.2 | — | — |
| Hydroxypropyl Guar | 0.2 | — | 0.3 | 0.2 | 0.2 | 0.2 | 0.3 | 0.1 | 0.2 | — |
| Hydroxypropyltrimonium | | | | | | | | | | |
| Chloride | | | | | | | | | | |
| Silicone Quaternium-22 | — | — | — | 0.5 | — | — | — | 0.5 | — | — |
| Dimethicone | — | 0.5 | — | — | — | — | — | 1.0 | — | — |
| Amodimethicone | — | 0.1 | — | — | 1.0 | 0.3 | — | — | 0.5 | — |
| Argania Spinosa Oil | — | — | 0.2 | 0.1 | — | 0.1 | 0.2 | — | — | — |
| Glycol Distearate | 0.5 | 0.5 | — | — | 0.5 | — | 0.3 | 0.5 | 0.5 | 0.5 |
| Acrylates/C10-30 Alkyl | 1.0 | — | — | — | 0.5 | — | — | — | 0.5 | — |
| Acrylate Crosspolymer | | | | | | | | | | |
| Sodium Hydroxide, 25% | 1.5 | — | — | — | 0.8 | — | — | — | 0.7 | — |
| Isostearamide MIPA; | — | — | — | — | — | 2.5 | — | — | 1.0 | 0.5 |
| Glycery lLaurate | | | | | | | | | | |
| Cocamide DEA | — | — | — | 1.0 | 0.5 | — | — | — | — | — |
| Sodium Chloride | 0.1 | 1.2 | — | 0.5 | — | 1.5 | — | 2.0 | — | 1.2 |
| PEG-120 Methyl Glucose | — | 3.5 | — | 0.2 | — | — | — | 0.6 | — | — |
| Dioleate | | | | | | | | | | |
| Xanthan Gum | — | — | 1.2 | — | — | 0.2 | 1.5 | — | — | — |
| Cellulose | — | — | — | 0.1 | — | 0.1 | 0.1 | — | 0.1 | — |
| Zinc Pyrithione | — | 0.1 | — | 1.1 | — | — | — | 0.5 | — | — |
| Benzophenone-4 | — | 0.1 | — | 0.2 | — | — | — | 0.2 | 0.1 | — |
| Tetrasodium EDTA | 0.1 | 0.1 | — | 0.1 | — | — | — | 0.1 | 0.1 | — |
| Caffeine | — | — | 0.1 | — | — | — | — | 0.1 | 0.1 | |
| Hydrolyzed Keratin | — | — | 0.1 | 0.1 | — | 0.1 | 0.2 | — | 0.1 | — |
| Panthenol | 0.1 | 0.1 | 0.1 | 0.1 | 0.1 | 0.1 | — | 0.1 | 0.1 | 0.1 |

| | |
|---|---|
| Citric Acid | ad pH |
| Parfums, Dyes, | q.s. |
| Preservatives | |

| | XI | XII | XIII | XIV | XV | XVI | XVII | XVIII | XIX | XX |
|---|---|---|---|---|---|---|---|---|---|---|
| Water | ad 100 | | | | | | | | | |
| SFX | 10.0 | 7.0 | 5.0 | 4.0 | 7.0 | 2.5 | 6.0 | 4.0 | 2.0 | 1.0 |
| Sodium Laureth Sulfate | — | — | 8.0 | — | — | — | — | 3.5 | — | — |
| Coco-Betaine | — | — | — | 5.5 | 5.0 | — | — | 3.0 | — | — |
| Cocamidopropyl Betaine | — | — | — | — | — | 6.0 | — | — | 3.0 | 4.0 |
| Sodium Cocoamphoacetate | — | — | — | — | 2.0 | — | — | — | — | — |
| Coco Glucoside | — | — | — | — | 2.0 | — | 4.0 | 1.5 | 6.0 | — |
| Glycolipids | — | — | — | — | — | — | 2.5 | 0.5 | 2.5 | 0.3 |
| Stearic Acid | — | 8.0 | — | — | — | — | — | — | — | 4.5 |
| Sodium Cocoyl Glycinate | — | — | — | — | — | 3.0 | 0.5 | — | — | 0.3 |
| Sodium Lauroyl Methyl | — | — | — | — | — | 1.0 | — | 1.0 | 2.0 | 0.5 |
| Isethionate | | | | | | | | | | |
| Sucrose Cocoate | 0.5 | 1.1 | — | 1.0 | 1.0 | — | — | 0.3 | 1.0 | 0.3 |
| Glycerin | 1.5 | 0.3 | 0.5 | — | 0.3 | 0.5 | 1.0 | 0.5 | — | 1.0 |
| PEG-7 Glyceryl Cocoate | — | — | 0.5 | — | — | 0.8 | — | 0.5 | — | 0.5 |
| PEG-40 Hydrogenated | — | — | 0.5 | — | — | 1.5 | — | 0.3 | — | — |
| Castor Oil | | | | | | | | | | |
| Polyglyceryl-4 Caprate | 0.5 | — | — | — | 0.4 | — | 0.5 | — | 1.1 | — |
| Polyquaternium-11 | — | 0.2 | — | — | 0.1 | 0.3 | — | 0.2 | — | — |
| Guar Hydroxypropyl- | 0.3 | — | 0.3 | 0.2 | 0.2 | — | 0.3 | 0.1 | 0.4 | 0.2 |
| trimonium Chloride | | | | | | | | | | |
| Silicone Quaternium-22 | — | — | 0.5 | — | — | — | — | 1.5 | — | — |
| PEG-12 Dimethicone | — | — | — | — | — | 1.5 | — | — | — | — |
| Dimethicone | — | — | 1.0 | — | 0.5 | — | — | — | — | — |
| Aminopropyl Dimethicone | — | — | — | — | 0.3 | — | — | — | — | — |
| Helianthus Annuus Oil | 0.1 | — | 0.2 | 0.1 | — | 0.1 | 0.3 | 0.2 | — | 0.1 |
| PEG-3 Distearate | — | — | 0.5 | — | — | 0.8 | — | 0.5 | — | — |
| Acrylates/C10-30 Alkyl | — | — | 0.2 | — | 0.5 | — | — | — | — | — |
| Acrylate Crosspolymer | | | | | | | | | | |
| Sodium Hydroxide, 25% | — | — | 0.3 | — | 0.8 | — | — | — | — | — |
| Cocamide DEA | — | — | — | — | — | 2.5 | — | — | — | 0.5 |
| Cocamide MEA | — | — | — | 1.0 | — | — | — | 1.0 | — | — |
| Sodium Chloride | 0.4 | 0.7 | 0.1 | — | — | 1.5 | — | 2.0 | — | 1.2 |
| Propylene Glycol (and) | — | 2.5 | 2.0 | — | — | 0.3 | — | 0.6 | — | 0.2 |
| PEG-55 | | | | | | | | | | |
| Propylene Glycol Oleate | | | | | | | | | | |
| Xanthan Gum | 1.2 | — | — | 1.0 | 0.2 | — | 1.5 | 0.1 | 1.1 | — |
| Hydroxyethyl Cellulose | 0.1 | — | — | 0.1 | — | 0.1 | 0.1 | — | — | — |
| Benzophenone-4 | — | 0.1 | 0.2 | — | — | 0.2 | — | 0.2 | — | 0.1 |
| Menthol | 0.1 | — | — | — | — | — | — | — | — | 0.1 |
| Tetrasodium Glutamate | — | 0.1 | — | 0.1 | 0.1 | — | 0.1 | 0.1 | — | 0.1 |
| Diacetate | | | | | | | | | | |
| Caffeine | 0.1 | — | 0.1 | — | — | — | — | 0.1 | — | — |
| Coumarin | 0.1 | — | — | 0.1 | 0.1 | — | 0.1 | 0.1 | — | 0.1 |
| Hydrolyzed Wheat Protein | 0.1 | — | 0.1 | 0.1 | — | — | 0.2 | 0.1 | 0.1 | 0.1 |
| Panthenol | 0.1 | 0.1 | — | 0.1 | 0.1 | 0.1 | 0.1 | — | 0.1 | 0.1 |

| | |
|---|---|
| Citric Acid | ad pH |
| Parfums, Dyes, | q.s. |
| Preservatives | |

| | XXI | XXII | XXIII | XIV | XXV | XXVI | XXVII | XXVIII | XXIX | XXX |
|---|---|---|---|---|---|---|---|---|---|---|
| Water | ad 100 | | | | | | | | | |
| SFX | 6.0 | 4.0 | 3.5 | 1.0 | 13.0 | 3.0 | 4.0 | 2.0 | 2.5 | 5.0 |
| Sodium Coco-Sulfate | 3.0 | 1.0 | — | 5.0 | 3.0 | 3.0 | 2.0 | — | 2.5 | 3.0 |
| Glycolipids | 1.0 | | 1.5 | 1.0 | | 2.0 | | — | 2.0 | — |
| Lauryl Glucoside | — | 2.0 | — | — | | — | — | 2.0 | 3.5 | 2.0 |
| Sodium Cocoyl Glutamate | 1.0 | — | — | — | 3.0 | — | — | — | | — |
| Sodium Cocoyl Glycinate | — | — | 1.5 | — | — | — | — | 2.5 | 1.5 | — |
| Sodium C14-16 Olefin | | | | | 3.0 | | 2.0 | — | — | 2.0 |
| Sulfonate | | | | | | | | | | |
| Lauroyl/Myristoyl Methyl | — | 1.0 | — | 2.0 | — | | — | — | — | — |
| Glucamide | | | | | | | | | | |
| Cocamidopropyl Betaine | — | 2.0 | 1.5 | 4.0 | — | 2.0 | — | 7.5 | — | — |
| Sodium Cocoamphoacetate | — | 2.0 | 5.0 | — | 2.0 | 2.0 | 4.0 | — | — | — |
| Sucrose Cocoate | 1.0 | — | 1.5 | — | 1.5 | 1.0 | — | 1.0 | — | 3.5 |
| Glycerin | 0.5 | 0.5 | — | — | 0.3 | — | 0.2 | — | 0.5− | — |
| Alcohol Denat. | — | — | 3.0 | — | — | 2.0 | — | — | — | — |
| Sorbitan Sesquicaprylate | 1.0 | — | 0.7 | 1.0 | | 0.5 | — | 0.5 | — | 1.0 |
| Isostearamide MIPA; | — | 1.0 | — | — | — | 2.0 | — | — | — | 0.5 |
| Glyceryl Laurate | | | | | | | | | | |
| Xanthan Gum | 1.0 | 0.7 | 0.5 | — | 0.7 | 0.3 | 0.7 | 0.5 | — | 0.5 |
| Chondrus Crispus | — | — | 1.5 | | 2.0 | 1.5 | 0.7 | — | — | 0.5 |
| Carrageenan | — | — | — | 1.0 | — | — | — | 0.3 | — | 0.5 |
| Hydroxypropyl Starch | — | — | — | — | — | — | — | 4.5 | 5.5 | — |
| Phosphate | | | | | | | | | | |
| Guar Hydroxpropyl- | 0.2 | — | 0.5 | 0.2 | 0.2 | 0.5 | 0.3 | 0.1 | — | — |
| trimonium Chloride | | | | | | | | | | |
| Bis-(Isostearoyl/ | 0.5 | 0.5 | — | — | 0.5 | — | — | — | 0.5 | — |
| Oleyl Isopropyl) | | | | | | | | | | |
| Dimonium Methosulfate | | | | | | | | | | |
| Prunus Amygdalus Dulcis | — | 0.1 | — | 0.1 | — | 0.05 | — | — | — | — |
| (Almond) Oil | | | | | | | | | | |
| Lemon Oil | | — | 0.2 | — | 0.1 | | 0.2 | | | |
| Cellulose | 0.5 | — | — | — | 0.7 | — | 0.3 | 0.1 | — | 0.5 |
| Charcoal Powder | 1.0 | — | — | — | — | — | — | — | — | — |
| Saccharum Officinarum | — | — | — | 0.1 | — | 0.2 | — | — | — | — |
| (Sugar Cane) Extract | | | | | | | | | | |
| Sodium Chloride | 0.7 | — | — | 0.5 | 1.0 | 0.7 | — | 2.0 | — | 1.2 |
| Polyglyceryl-6 Caprylate; | | 3.0 | — | 4.0 | | 2.5 | — | | | |
| Polyglyceryl-3 Cocoate; | | | | | | | | | | |
| Polyglyceryl-4 Caprate; | | | | | | | | | | |
| Polyglyceryl-6 Ricinoleate | | | | | | | | | | |
| Caprylyl/Capryl Glucoside; | | — | 3.0 | — | 1.0 | | 2.0 | | | |
| Aqua, Sodium Cocoyl | | | | | | | | | | |
| Glutamate; Glyceryl | | | | | | | | | | |
| Caprylate; Ccitric Acid; | | | | | | | | | | |
| Polyglyceryl-6 | | | | | | | | | | |
| Oleate, Sodium Surfactin | | | | | | | | | | |
| Tocopherol | — | 0.1 | — | 0.1 | — | 0.1 | 0.1 | — | 0.1 | — |
| Phytic Acid | 0.1 | 0.1 | — | — | — | — | — | 0.1 | 0.1 | — |

| | |
|---|---|
| Citric Acid | ad pH |
| Parfums, Dyes, | q.s. |
| Preservatives | |

| | XXXI | XXXII | XXXIII | XXXIV | XXXV | XXXVI | XXXVII | XXXVIII |
|---|---|---|---|---|---|---|---|---|
| Water | ad 100 | | | | | | | |
| SFX | 1.0 | 1.5 | 2.0 | 1.5 | 3.0 | 2.5 | 4.0 | 5.0 |
| Capryl/Capramidopropyl | — | 2.0 | — | — | 1.0 | — | 2.0 | |
| Betaine | | | | | | | | |
| Glycolipids | 1.0 | — | — | — | 1.0 | — | — | |
| Decyl Glucoside | 1.0 | — | — | — | — | 2.0 | — | |
| Disodium | — | — | 3.0 | 2.0 | — | — | 2.0 | |
| Cocoamphodiacetate | | | | | | | | |
| PEG-6 Caprylic/Capric | 3.0 | 1.5 | — | — | — | — | — | 2.0 |
| Glycerides | | | | | | | | |
| Polyglyceryl-6 Caprylate; | | — | 2.0 | — | — | 3.5 | — | |
| Polyglyceryl-4 Caprate | | | | | | | | |
| Polyglyceryl-6 Caprylate; | — | 3.0 | | — | 4.0 | — | — | |
| Polyglyceryl-3 Cocoate; | | | | | | | | |
| Polyglyceryl-4 Caprate; | | | | | | | | |
| Polyglyceryl-6 | | | | | | | | |
| Ricinoleate | | | | | | | | |
| Polyglyceryl-4 Caprate | — | — | | 2.5 | | — | — | |
| Persea Gratissima | — | 0.1 | | — | 0.2 | — | 0.1 | 0.05 |
| (Avocado) Oil | | | | | | | | |
| Rosemary Oil | 0.1 | — | 0.05 | — | 0.1 | — | — | |
| Hydroxypropyl | — | 0.5 | | — | — | — | 0.5 | |
| Methylcellulose | | | | | | | | |
| Sorbitan Sesquicaprylate | 0.3 | — | | 0.5 | — | 0.3 | 0.5 | 0.1 |
| Betaine | — | 0.5 | | 1.0 | — | 1.0 | — | 1.0 |
| Allantoin | — | — | 0.1 | — | 0.1 | | — | |
| Starch | — | — | — | 0.1 | 0.05 | | — | 0.1 |
| Glycerin | 3.0 | 1.0 | — | — | — | 1.0 | 1.0 | — |
| Hexylene Glycol | 1.0 | — | 1.0 | — | 1.0 | 1.0 | — | — |
| Methylpropanediol; | 1.2 | — | — | — | — | — | — | — |
| Caprylyl Glycol; Benzoic | | | | | | | | |
| Acid | | | | | | | | |
| Benzyl Alcohol, Caprylyl | — | 1.0 | 1.0 | 1.0 | 1.0 | — | — | — |
| Glycol, Benzoic Acid | | | | | | | | |
| Aqua, Sodium | — | — | — | — | — | 1.0 | 1.0 | 1. |
| Levulinate, Sodium | | | | | | | | |
| Benzoate | | | | | | | | |

| | |
|---|---|
| pH adjuster (Citric Acid | ad pH |
| or NaOH) | |
| Preservatives, Parfum | q.s. |

| Ingredient | Pouch 1 | Pouch 2 | Pouch 3 |
|---|---|---|---|
| SFX | 15 | 10 | 20 |
| Sodium laureth sulfate | 0 | 0 | 10 |
| Fatty alcohol ethoxylate | 14 | 14 | 14 |
| (C12-14, 7EO) | | | |
| Fatty acid (C8-C18) | 13 | 13 | 13 |
| 1,2-propane diol | 22 | 22 | 22 |
| Glycerine | 22 | 22 | 22 |
| Sodium Hydroxide | 4 | 4 | 4 |
| Citric acid | to pH 8 | to pH 8 | to pH 8 |
| Medley ® Brilliant 300 L | 3 | 3 | 3 |
| Water | Add to 100% | Add to 100% | Add to 100% |

| Ingredient | Pouch 4 | Pouch 5 | Pouch 6 |
|---|---|---|---|
| SFX | 15 | 10 | 5 |
| Persicomycin | 0.1 | 0.1 | 0.1 |
| Sodium laureth sulfate | 0 | 0 | 10 |
| Fatty alcohol ethoxylate | 14 | 14 | 14 |
| (C12-14, 3EO) | | | |
| Fatty acid (C8-C18) | 13 | 13 | 13 |
| 1,2-propane diol | 22 | 22 | 22 |
| Glycerine | 22 | 22 | 22 |
| Sodium Hydroxide | 4 | 4 | 4 |
| Citric acid | to pH 8 | to pH 8 | to pH 8 |
| Medley ® Brilliant 300 L | 3 | 3 | 3 |
| Water | Add to 100% | Add to 100% | Add to 100% |

| Ingredient | Pouch 7 | Pouch 8 | Pouch 9 |
|---|---|---|---|
| SFX | 15 | 10 | 5 |
| Sodium laureth sulfate | 0 | 0 | 10 |
| Fatty alcohol ethoxylate | 14 | 14 | 14 |
| (C12-14, 3EO) | | | |
| Fatty acid (C8-C18) | 13 | 13 | 13 |
| 1,2-propane diol | 22 | 22 | 22 |
| Glycerine | 22 | 22 | 22 |
| Sodium Hydroxide | 4 | 4 | 4 |
| Citric acid | to pH 8 | to pH 8 | to pH 8 |
| Medley ® Brilliant 300 L | 3 | 3 | 3 |
| Water | Add to 100% | Add to 100% | Add to 100% |

| Ingredient | Pouch 10 | Pouch 11 | Pouch 12 |
|---|---|---|---|
| SFX | 15 | 10 | 5 |
| Sodium laureth sulfate | 0 | 0 | 10 |
| Fatty alcohol ethoxylate | 14 | 14 | 14 |
| (C12-14, 7EO) | | | |
| Fatty acid (C8-C18) | 13 | 13 | 13 |
| 1,2-propane diol | 22 | 22 | 22 |
| Glycerine | 22 | 22 | 22 |
| Sodium Hydroxide | 4 | 4 | 4 |
| Citric acid | to pH 8 | to pH 8 | to pH 8 |
| Medley ® Brilliant 300 L | 3 | 3 | 3 |
| Water | Add to 100% | Add to 100% | Add to 100% |

| Ingredients | Detergent | Detergent | Detergent |
|---|---|---|---|
| Na soap from palm | 2 | 2 | 2 |
| kernel oil | | | |
| Sodium Citrate | 6 | 6 | 6 |
| SFX | 15 | 15 | 15 |
| SLES 3EO | 5 | 5 | |
| FAEO C12-18, 7 EO | 5 | | 5 |
| Dissolvine ® GLDA | 1 | 1 | 1 |
| FAEO C12-18, 3 EO | | 5 | 6 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent | Detergent | Detergent |
|---|---|---|---|
| Na soap from palm | 2 | 2 | 2 |
| kernel oil | | | |
| Sodium Citrate | 6 | 6 | 6 |
| SFX | 15 | 10 | 20 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent | Detergent | Detergent |
|---|---|---|---|
| Na soap from palm | 6 | 6 | 6 |
| kernel oil | | | |
| Sodium Citrate | 6 | 6 | 6 |
| SFX | 15 | 10 | 20 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent | Detergent | Detergent |
|---|---|---|---|
| Na soap from palm | 2 | 2 | 2 |
| kernel oil | | | |
| Sodium Citrate | 6 | 6 | 6 |
| SFX | 15 | 10 | 20 |
| Dissolvine ® GLDA | 1 | 1 | 1 |
| Phenoxyethanol | 0.45 | 0.45 | 0.45 |
| NaOH (water solution) | 1 | 1 | 1 |
| Polymers (soil release, | 1 | 1 | 1 |
| dye transfer inhibitors, | | | |
| rheology polymers, | | | |
| anti-redeposition, | | | |
| polycarboxylates, | | | |
| polyacrylates, | | | |
| polyvinyl alcohol, | | | |
| polyethylene glycol, | | | |
| Polyethyleneimine | | | |
| Ethoxylated) | | | |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent | Detergent | Detergent |
|---|---|---|---|
| Na soap from palm | 2 | 2 | 2 |
| kernel oil | | | |
| Sodium Citrate | 6 | 6 | 6 |
| SFX | 15 | 10 | 20 |
| Dissolvine ® GLDA | 1 | 1 | 1 |
| Phenoxyethanol | 0.45 | 0.45 | 0.45 |
| NaOH (water solution) | 1 | 1 | 1 |
| Miscellaneous (fragrances, | 1 | 1 | 1 |
| preservatives, dyes, | | | |
| pigments, Bitrex, sodium | | | |
| formate, sorbitol, | | | |
| ethoxylated M-toluidyne, | | | |
| antifoaming agent, | | | |
| defoamers, solvents, | | | |
| trimethylsiloxysilicate, | | | |
| etylhexyl stearate, | | | |
| sulfated ethoxylated | | | |
| hexamethylenediamine | | | |
| quaternized, 2- | | | |
| aminoethanol | | | |
| sulfate (salt)) | | | |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent | Detergent | Detergent |
|---|---|---|---|
| Na soap from palm | 3.85 | 3.85 | 3.85 |
| kernel oil | | | |
| SFX | 15 | 10 | 20 |
| SLES 3EO | 5 | 5 | 5 |
| C8C 16-Alkyl | 5 | 5 | 5 |
| polyglucoside | | | |
| Monoethanolamine | 5.95 | 5.95 | 5.95 |
| Trilon M | 2 | 2 | 2 |
| Citric acid | 3 | 3 | 3 |
| Medley ® Brilliant 300 L | 2 | 2 | 2 |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent | Detergent | Detergent |
|---|---|---|---|
| Na soap from palm | 3.85 | 3.85 | 3.85 |
| kernel oil | | | |
| SFX | 15 | 10 | 20 |
| SLES 3EO | 5 | 5 | 5 |
| C10C16-Alkyl | 5 | 5 | 5 |
| polyglucoside | | | |
| Monoethanolamine | 5.95 | 5.95 | 5.95 |
| Trilon M | 2 | 2 | 2 |
| Citric acid | 3 | 3 | 3 |
| Medley ® Brilliant 300 L | 2 | 2 | 2 |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent | Detergent | Detergent |
|---|---|---|---|
| Na soap from palm | 3.85 | 3.85 | 3.85 |
| kernel oil | | | |
| SFX | 15 | 10 | 20 |
| SLES 3EO | 5 | 5 | 5 |
| C8C 16-Alkyl | 5 | 5 | 5 |
| polyglucoside | | | |
| Monoethanolamine | 5.95 | 5.95 | 5.95 |
| Trilon M | 2 | 2 | 2 |
| WeylClean PLA 1 | 1 | 1 | 1 |
| Citric acid | 3 | 3 | 3 |
| Medley ® Brilliant 300 L | 2 | 2 | 2 |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent | Detergent | Detergent |
|---|---|---|---|
| Na soap from palm | 3.85 | 3.85 | 3.85 |
| kernel oil | | | |
| SFX | 15 | 10 | 20 |
| SLES 3EO | 5 | 5 | 5 |
| REWOTERIC AM C | 5 | 5 | 5 |
| Monoethanolamine | 5.95 | 5.95 | 5.95 |
| Trilon M | 2 | 2 | 2 |
| WeylClean PLA 1 | 1 | 1 | 1 |
| Citric acid | 3 | 3 | 3 |
| Medley ® Brilliant 300 L | 2 | 2 | 2 |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent | Detergent | Detergent |
|---|---|---|---|
| Na soap from palm | 2 | 2 | 2 |
| kernel oil | | | |
| Sodium Citrate | 6 | 6 | 6 |
| SFX | 15 | 10 | 20 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 1 | Detergent 2 | Detergent 3 |
|---|---|---|---|
| Na soap from palm | 2 | 2 | 2 |
| kernel oil | | | |
| Sodium Citrate | 6 | 6 | 6 |
| SFX | 15 | 10 | 20 |
| SLES 3EO | 5 | 5 | 5 |
| FAEO C12-18, 7 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 4 | Detergent 5 | Detergent 6 |
|---|---|---|---|
| Na soap from palm | 2 | 2 | 2 |
| kernel oil | | | |
| Sodium Citrate | 6 | 6 | 6 |
| SFX | 15 | 10 | 20 |
| persicomycin | 0.1 | 0.1 | 0.1 |
| SLES 3EO | 5 | 5 | 5 |
| FAEO C12-18, 3 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 7 | Detergent 8 | Detergent 9 |
|---|---|---|---|
| Na soap from palm | 2 | 2 | 2 |
| kernel oil | | | |
| Sodium Citrate | 6 | 6 | 6 |
| SFX | 15 | 10 | 20 |
| SLES 3EO | 5 | 5 | 5 |
| FAEO C12-18, 4 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 10 | Detergent 11 | Detergent 12 |
|---|---|---|---|
| Na soap from palm kernel oil | 2 | 2 | 2 |
| Sodium Citrate | 6 | 6 | 6 |
| SFX | 15 | 10 | 20 |
| FAEO C12-18, 4 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 13 | Detergent 14 | Detergent 15 |
|---|---|---|---|
| Na soap from palm kernel oil | 2 | 2 | 2 |
| Sodium Citrate | 6 | 6 | 6 |
| SFX | 15 | 10 | 20 |
| SLES 3EO | 5 | 5 | 5 |
| Glucopon USP 600 (APG) | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 16 | Detergent 17 | Detergent 18 |
|---|---|---|---|
| Na soap from palm kernel oil | 2 | 2 | 2 |
| Sodium Citrate | 6 | 6 | 6 |
| SFX | 15 | 10 | 20 |
| Glucopon USP 600 (APG) | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 19 | Detergent 20 | Detergent 21 |
|---|---|---|---|
| Na soap from palm kernel oil | 2 | 2 | 2 |
| Sodium Citrate | 6 | 6 | 6 |
| SFX | 15 | 10 | 20 |
| Myristic acid | 2.5 | 2.5 | 2.5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 22 | Detergent 23 | Detergent 24 |
|---|---|---|---|
| Na soap from palm kernel oil | 2 | 2 | 2 |
| Sodium Citrate | 6 | 6 | 6 |
| SFX | 15 | 10 | 20 |
| Lauric acid | 2.5 | 2.5 | 2.5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 25 | Detergent 26 | Detergent 27 |
|---|---|---|---|
| Na soap from palm kernel oil | 2 | 2 | 2 |
| Sodium Citrate | 6 | 6 | 6 |
| SFX | 15 | 10 | 20 |
| SLES 3EO | 5 | 5 | 5 |
| FAEO C12-18, 5 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 28 | Detergent 29 | Detergent 30 |
|---|---|---|---|
| Na soap from palm kernel oil | 2 | 2 | 2 |
| Sodium Citrate | 6 | 6 | 6 |
| SFX | 15 | 10 | 20 |
| SLES 3EO | 5 | 5 | 5 |
| REWOTERIC AM V | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 31 | Detergent 32 | Detergent 33 |
|---|---|---|---|
| Na soap from palm kernel oil | 2 | 2 | 2 |
| Sodium Citrate | 6 | 6 | 6 |
| SFX | 15 | 10 | 20 |
| REWOTERIC AM V | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |
| Ingredients | Detergent 34 | Detergent 35 | Detergent 36 |
| Na soap from palm kernel oil | 2 | 2 | 2 |
| Sodium Citrate | 6 | 6 | 6 |
| SFX | 15 | 10 | 20 |
| SLES 3EO | 5 | 5 | 5 |
| Neutrol ® MGDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |
| Ingredients | Detergent 34 | Detergent 35 | Detergent 36 |
| Na soap from palm kernel oil | 2 | 2 | 2 |
| Sodium Citrate | 6 | 6 | 6 |
| SFX | 15 | 10 | 20 |
| SLES 3EO | 5 | 5 | 5 |
| FAEO C12-18, 4 EO | 3 | 3 | 3 |
| Trilon ® M | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 37 | Detergent 38 | Detergent 39 |
|---|---|---|---|
| Na soap from palm kernel oil | 2 | 2 | 2 |
| Sodium Citrate | 6 | 6 | 6 |
| SFX | 15 | 10 | 20 |
| SLES 3EO | 5 | 5 | 5 |
| Glucopon ®215P | 3 | 3 | 3 |
| Trilon ® M | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |
| Ingredients | Detergent 40 | Detergent 41 | Detergent 42 |
| Na soap from palm kernel oil | 2 | 2 | 2 |
| Sodium Citrate | 6 | 6 | 6 |
| SFX | 15 | 10 | 20 |
| Glucopon ®215P | 5 | 5 | 5 |
| Trilon ® M | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 43 | Detergent 44 | Detergent 45 |
|---|---|---|---|
| Na soap from palm kernel oil | 2 | 2 | 2 |
| Sodium Citrate | 6 | 6 | 6 |
| SFX | 15 | 10 | 20 |
| Pelargonic acid | 5 | 5 | 5 |
| Trilon ® M | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 46 | Detergent 47 | Detergent 48 |
|---|---|---|---|
| Na soap from palm kernel oil | 2 | 2 | 2 |
| Sodium Citrate | 6 | 6 | 6 |
| SFX | 15 | 10 | 20 |
| Oleic acid | 5 | 5 | 5 |
| Trilon ® M | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 49 | Detergent 50 | Detergent 51 |
|---|---|---|---|
| Na soap from palm kernel oil | 2 | 2 | 2 |
| Sodium Citrate | 6 | 6 | 6 |
| SFX | 15 | 10 | 20 |
| Palmitic acid | 5 | 5 | 5 |
| Trilon ® M | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 49 | Detergent 50 | Detergent 51 |
|---|---|---|---|
| MEA soap from palm kernel | 2 | 2 | 2 |
| oil | | | |
| Sodium Citrate | 6 | 6 | 6 |
| SFX | 15 | 10 | 20 |
| SLES | 5 | 5 | 5 |
| Trilon ® M | 1 | 1 | 1 |
| Phenoxyethanol | 0.45 | 0.45 | 0.45 |
| Monoethanol amine | 1.1 | 1.1 | 1.1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 52 | Detergent 53 | Detergent 54 |
|---|---|---|---|
| MEA soap from palm kernel | 2 | 2 | 2 |
| oil | | | |
| Sodium Citrate | 6 | 6 | 6 |
| SFX | 15 | 10 | 20 |
| SLES | 5 | 5 | 5 |
| FAEO C12-18, 4 EO | 3 | 3 | 3 |
| Trilon ® M | 1 | 1 | 1 |
| Phenoxyethanol | 0.45 | 0.45 | 0.45 |
| Monoethanol amine | 1.1 | 1.1 | 1.1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| Ingredients | Detergent 52 | Detergent 53 | Detergent 54 |
|---|---|---|---|
| MEA soap from palm kernel | 2 | 2 | 2 |
| oil | | | |
| Sodium Citrate | 6 | 6 | 6 |
| SFX | 15 | 10 | 20 |
| SLES | 5 | 5 | 5 |
| Myristic acid | 3 | 3 | 3 |
| Trilon ® M | 1 | 1 | 1 |
| Phenoxyethanol | 0.45 | 0.45 | 0.45 |
| Monoethanol amine | 1.1 | 1.1 | 1.1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 55 | 56 | 57 |
| MEA soap from palm kernel oil | 2 | 2 | 2 |
| Sodium Citrate | 6 | 6 | 6 |
| SFX | 15 | 10 | 20 |
| SLES | 5 | 5 | 5 |
| Glucopon 600 | 5 | 5 | 5 |
| Trilon ® M | 1 | 1 | 1 |
| Phenoxyethanol | 0.45 | 0.45 | 0.45 |
| Monoethanol amine | 1.1 | 1.1 | 1.1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 58 | 59 | 60 |
| MEA soap from palm kernel oil | 2 | 2 | 2 |
| Sodium Citrate | 6 | 6 | 6 |
| SFX | 15 | 10 | 20 |
| Glucopon 600 | 5 | 5 | 5 |
| Trilon ® M | 1 | 1 | 1 |
| Phenoxyethanol | 0.45 | 0.45 | 0.45 |
| Monoethanol amine | 1.1 | 1.1 | 1.1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 61 | 62 | 63 |
| MEA soap from palm kernel oil | 2 | 2 | 2 |
| MEA Citrate | 6 | 6 | 6 |
| SFX | 15 | 10 | 20 |
| Glucopon 600 | 5 | 5 | 5 |
| Trilon ® M | 1 | 1 | 1 |
| Phenoxyethanol | 0.45 | 0.45 | 0.45 |
| Monoethanol amine | 1.1 | 1.1 | 1.1 |
| Medley@ Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 64 | 65 | 66 |
| Hydrogen peroxide | 5 | 5 | 5 |
| Citric Acid | 2 | 2 | 2 |
| SFX | 15 | 10 | 20 |
| DIETHYLENETRIAMINE | 0.1 | 0.1 | 0.1 |
| PENTAMETHYLPHOSPHONATE | | | |
| DISODIUM DISTYRYL | 0.2 | 0.2 | 0.2 |
| BIPHENYL DISULFONATE | | | |
| | Add water | Add water | Add water |
| | to 100% | to 100% | to 100% |
| pH | 5.0 | 5.0 | 5.0 |

| | Detergent | Detergent | Detergent |
|---|---|---|---|
| Ingredients | 67 | 68 | 69 |
| Hydrogen peroxide | 5 | 5 | 5 |
| Citric Acid | 2 | 2 | 2 |
| di-rhamnolipids | 10 | — | — |
| FAEO C12-18, 3 EO | 2 | 2 | 2 |
| Glucopon 600 | 2 | 2 | 2 |
| SLES | 2 | 2 | 2 |
| DIETHYLENETRIAMINE | 0.1 | 0.1 | 0.1 |
| PENTAMETHYLPHOSPHONATE | | | |
| DISODIUM DISTYRYL | 0.2 | 0.2 | 0.2 |
| BIPHENYL DISULFONATE | | | |
| | Add water | Add water | Add water |
| | to 100% | to 100% | to 100% |
| pH | 5.0 | 5.0 | 5.0 |

| | Detergent | Detergent | Detergent |
|---|---|---|---|
| Ingredients | 70 | 71 | 72 |
| MEA soap from palm kernel oil | 2 | 2 | 2 |
| Sodium Citrate | 6 | 6 | 6 |
| SFX | 15 | 10 | 20 |
| persicomycin | 0.1 | 0.1 | 0.1 |
| SLES | 5 | 5 | 5 |
| FAEO C12-18, 4 EO | 3 | 3 | 3 |
| Trilon ® M | 1 | 1 | 1 |
| Phenoxyethanol | 0.45 | 0.45 | 0.45 |
| KOH (aqueous solution) | 1.1 | 1.1 | 1.1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 73 | 74 | 75 |
| MEA soap from palm kernel oil | 2 | 2 | 2 |
| Sodium Citrate | 6 | 6 | 6 |
| SFX | 15 | 10 | 20 |
| SLES | 5 | 5 | 5 |
| FAEO C12-18, 4 EO | 3 | 3 | 3 |
| Trilon ® M | 1 | 1 | 1 |
| Phenoxyethanol | 0.45 | 0.45 | 0.45 |
| Triathanolamine | 1.1 | 1.1 | 1.1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 74 | 75 | 76 |
| Na soap from palm kernel oil | 2 | 2 | 2 |
| Sodium Citrate | 6 | 6 | 6 |
| SFX | 15 | 10 | 5 |
| SLES 3EO | 5 | 5 | 5 |
| FAEO C12-18, 7 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Enzymes cocktail (proteases, | 2 | 2 | 2 |
| amylases, lipases, mannanases, | | | |
| pecktinases, cellulalase, | | | |
| phosphodiesterases) | | | |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 77 | 78 | 79 |
| Na soap from palm kernel oil | 2 | 2 | 2 |
| Sodium Citrate | 6 | 6 | 6 |
| SFX | 15 | 10 | 20 |
| SLES 3EO | 5 | 5 | 5 |
| FAEO C12-18, 7 EO | 5 | 5 | 5 |
| Dissolvine ® GLDA | 1 | 1 | 1 |
| Polymers (soil release, dye transfer inhibitors, | 1 | 1 | 1 |
| rheology polymers, anti-redeposition, | | | |
| polycarboxylates, polyacrylates, polyvinyl alcohol, | | | |
| polyethylene glycol, Polyethyleneimine | | | |
| Ethoxylated) | | | |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 80 | 81 | 82 |
| Na soap from palm kernel oil | 2 | 2 | 2 |
| Sodium Citrate | 6 | 6 | 6 |
| SFX | 15 | 10 | 20 |
| SLES 3EO | 5 | 5 | 5 |
| FAEO C12-18, 7 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Solvents (ethanol, glycerine, | 10 | 10 | 10 |
| 1,2-propanediol) | | | |
| Medley ® Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 83 | 84 | 85 |
| Na soap from palm kernel oil | 2 | 2 | 2 |
| Sodium Citrate | 6 | 6 | 6 |
| SFX | 15 | 10 | 20 |
| SLES 3EO | 5 | 5 | 5 |
| FAEO C12-18, 7 EO | 5 | 5 | 5 |
| Builders (phosphonates, | 1 | 1 | 1 |
| Tetrasodium glutamate | | | |
| diacetate, Trisodium | | | |
| dicarboxymethyl alaninate, | | | |
| monoethanolamine etidronate) | | | |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 86 | 87 | 88 |
| Na soap from palm kernel oil | 2 | 2 | 2 |
| Sodium Citrate | 6 | 6 | 6 |
| SFX | 15 | 10 | 20 |
| SLES 3EO | 5 | 5 | 5 |
| FAEO C12-18, 7 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| Miscellaneous (fragrances, preservatives, dyes, | 1 | 1 | 1 |
| pigments, Bitrex, sodium formate, sorbitol, ethoxylated | | | |
| M-toluidyne, antifoaming agent, | | | |
| trimethylsiloxysilicate, defoamers, solvents, Ethanol, | | | |
| porpylene glycol, monopropalene glycol, etylhexyl | | | |
| stearate, sulfated ethoxylated hexamethylenediamine | | | |
| quaternized, 2-aminoethanol sulfate (salt)) | | | |
| | Add water | Add water | Add water |
| | to 100% | to 100% | to 100% |
| pH | 8.0 | 8.0 | 8.0 |

| | Detergent | Detergent | Detergent |
|---|---|---|---|
| Ingredients | 89 | 90 | 91 |
| Na soap from palm kernel oil | 2 | 2 | 2 |
| Sodium Citrate | 6 | 6 | 6 |
| SFX | 15 | 10 | 20 |
| SLES 3EO | 5 | 5 | 5 |
| FAEO C12-18, 7 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| Inorganic salts (sodium chloride, | 0.2 | 0.2 | 0.2 |
| magnesium chloride, potassium | | | |
| chloride, potassium sulfite, | | | |
| sodium sulfite, calcium chloride) | | | |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 92 | 93 | 94 |
| MEA soap from palm kernel oil | 2 | 2 | 2 |
| Sodium Citrate | 6 | 6 | 6 |
| SFX | 15 | 10 | 20 |
| SLES | 5 | 5 | 5 |
| Trilon ® M | 1 | 1 | 1 |
| Phenoxyethanol | 0.45 | 0.45 | 0.45 |
| Diethanol amine | 1.1 | 1.1 | 1.1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 95 | 96 | 97 |
| Na soap from palm kernel oil | 2 | 2 | 2 |
| Sodium Citrate | 6 | 6 | 6 |
| SFX | 15 | 10 | 20 |
| SLES 3EO | 5 | 5 | 5 |
| Sorbitan Sesquioctanoate | 1 | 1 | 1 |
| FAEO C12-18, 7 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 98 | 99 | 100 |
| Fatty acid, saturated (Propanoic acid, Butanoic acid, | 2 | 2 | 2 |
| Pentanoic acid, Hexanoic acid, Heptanoic acid, | | | |
| Octanoic acid, Nonanoic acid, Decanoic acid, | | | |
| Undecanoic acid, Dodecanoic acid, Tridecanoic acid, | | | |
| Tetradecanoic acid, Pentadecanoic acid, | | | |
| Hexadecanoic acid, Heptadecanoic acid, | | | |
| Nonadecanoic acid, Eicosanoic acid) | | | |
| Sodium Citrate | 6 | 6 | 6 |
| SFX | 15 | 10 | 20 |
| SLES 3EO | 5 | 5 | 5 |
| Sorbitan Sesquioctanoate | 1 | 1 | 1 |
| FAEO C12-18, 7 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 101 | 102 | 103 |
| Fatty acid, unsaturated (myristoleic acid, palmitoleic | 2 | 2 | 2 |
| acid, sapienic acid, oleic acid, elaidic acid, vaccenic | | | |
| acid, linoleic acid, linoelaidic acid, alpha-linolenic | | | |
| acid, arachidonic acid, erucic acid, docosahexaenoic | | | |
| acid, and eicosapentaenoic acid) | | | |
| Sodium Citrate | 6 | 6 | 6 |
| SFX | 15 | 10 | 20 |
| SLES 3EO | 5 | 5 | 5 |
| FAEO C12-18, 7 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 104 | 105 | 106 |
| Na soap from palm | 2 | 2 | 2 |
| kernel oil | | | |
| Sodium Citrate | 6 | 6 | 6 |
| SFX | 15 | 10 | 20 |
| SLES 3EO | 5 | 5 | 5 |
| FAEO C12-18, 7 EO | 5 | 5 | 5 |
| Dissolvine ® GLDA | 1 | 1 | 1 |
| Phenoxyethanol | 0.45 | 0.45 | 0.45 |
| NaOH (water solution) | 3 | 3 | 3 |
| Medley ® Brilliant | 2 | 2 | 2 |
| 300 L | Add water | Add water | Add water |
| | to 100% | to 100% | to 100% |
| pH | 9.5 | 9.5 | 9.5 |

| | Detergent | Detergent | Detergent |
|---|---|---|---|
| Ingredients | 104 | 105 | 106 |
| Fatty acid, unsaturated (myristoleic acid, palmitoleic | 6 | 6 | 6 |
| acid, sapienic acid, oleic acid, elaidic acid, vaccenic | | | |
| acid, linoleic acid, linoelaidic acid, alpha-linolenic | | | |
| acid, arachidonic acid, erucic acid, docosahexaenoic | | | |
| acid, and eicosapentaenoic acid) | | | |
| Sodium Citrate | 6 | 6 | 6 |
| SFX | 15 | 10 | 20 |
| SLES 3EO | 5 | 5 | 5 |
| FAEO C12-18, 7 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 107 | 108 | 109 |
| Fatty acid, unsaturated (myristoleic acid, palmitoleic | 2 | 2 | 2 |
| acid, sapienic acid, oleic acid, elaidic acid, vaccenic | | | |
| acid, linoleic acid, linoelaidic acid, alpha-linolenic | | | |
| acid, arachidonic acid, erucic acid, docosahexaenoic | | | |
| acid, and eicosapentaenoic acid) | | | |
| Sodium Citrate | 6 | 6 | 6 |
| SFX | 15 | 10 | 20 |
| persicomycin | 0.1 | 0.1 | 0.1 |
| SLES 3EO | 5 | 5 | 5 |
| FAEO C12-18, 4 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant 300 L | 2 | 2 | 2 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 110 | 111 | 112 |
| Na soap from | 2 | 2 | 2 |
| palm kernel oil | | | |
| Sodium Citrate | 6 | 6 | 6 |
| SFX | 15 | 10 | 20 |
| SLES 3EO | 5 | 5 | 5 |
| FAEO C12-18, 3 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 113 | 114 | 115 |
| Na soap from | 3.85 | 3.85 | 3.85 |
| palm kernel oil | | | |
| Sodium Citrate | 6 | 6 | 6 |
| di-rhamnolipids | SFX | 15 | 10 |
| SLES 3EO | 5 | 5 | 5 |
| Monoethanolamine | 5.95 | 5.95 | 5.95 |
| Trilon M | 2 | 2 | 2 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 116 | 117 | 118 |
| Na soap from | 3.85 | 3.85 | 3.85 |
| palm kernel oil | | | |
| SFX | 15 | 10 | 20 |
| Monoethanolamine | 5.95 | 5.95 | 5.95 |
| Trilon M | 2 | 2 | 2 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 119 | 120 | 121 |
| Na soap from | 3.85 | 3.85 | 3.85 |
| palm kernel oil | | | |
| Sodium Citrate | 6 | 6 | 6 |
| SFX | 15 | 10 | 20 |
| SLES 3EO | 5 | 5 | 5 |
| Monoethanolamine | 5.95 | 5.95 | 5.95 |
| Trilon M | 2 | 2 | 2 |
| WeylClean PLA 1 | 1 | 1 | 1 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 122 | 123 | 124 |
| Na soap from | 3.85 | 3.85 | 3.85 |
| palm kernel oil | | | |
| SFX | 15 | 10 | 20 |
| SLES 3EO | 5 | 5 | 5 |
| Monoethanolamine | 5.95 | 5.95 | 5.95 |
| Trilon M | 2 | 2 | 2 |
| Texacare SRN | 1 | 1 | 1 |
| 170 Terra | | | |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 125 | 126 | 127 |
| Na soap from | 3.85 | 3.85 | 3.85 |
| palm kernel oil | | | |
| di-rhamnolipids | 15 | — | — |
| mono-rhamnolipids | — | 15 | — |
| glucolipids | — | — | 15 |
| persicomycin | 0.1 | 0.1 | 0.1 |
| SLES 3EO | 5 | 5 | 5 |
| Monoethanolamine | 5.95 | 5.95 | 5.95 |
| Trilon M | 2 | 2 | 2 |
| Na-Carboxymethylinnulin | 1 | 1 | 1 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 128 | 129 | 130 |
| Na soap from | 3.85 | 3.85 | 3.85 |
| palm kernel oil | | | |
| SFX | 15 | 10 | 20 |
| SLES 3EO | 5 | 5 | 5 |
| Monoethanolamine | 5.95 | 5.95 | 5.95 |
| Trilon M | 2 | 2 | 2 |
| FAEO C12-18, 4 EO | 5 | 5 | 5 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| | Detergent | Detergent | Detergent |
|---|---|---|---|
| Ingredients | 131 | 132 | 133 |
| Na soap from | 3.85 | 3.85 | 3.85 |
| palm kernel oil | | | |
| SFX | 15 | 10 | 20 |
| SLES 3EO | 5 | 5 | 5 |
| Monoethanolamine | 5.95 | 5.95 | 5.95 |
| Trilon M | 2 | 2 | 2 |
| FAEO C12-18, 7 EO | 5 | 5 | 5 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent 134 | Detergent 135 | Detergent 136 |
|---|---|---|---|
| Na soap from palm | 3.85 | 3.85 | 3.85 |
| kernel oil | | | |
| SFX | 15 | 10 | 20 |
| SLES 3EO | 5 | 5 | 5 |
| Monoethanolamine | 5.95 | 5.95 | 5.95 |
| Trilon M | 2 | 2 | 2 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent 137 | Detergent 138 | Detergent 139 |
|---|---|---|---|
| Na soap from palm | 3.85 | 3.85 | 3.85 |
| kernel oil | | | |
| SFX | 15 | 10 | 20 |
| SLES 3EO | 5 | 5 | 5 |
| Monoethanolamine | 5.95 | 5.95 | 5.95 |
| Trilon M | 2 | 2 | 2 |
| Citric acid | 6 | 6 | 6 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent 140 | Detergent 141 | Detergent 142 |
|---|---|---|---|
| Na soap from palm | 3.85 | 3.85 | 3.85 |
| kernel oil | | | |
| SFX | 15 | 10 | 20 |
| SLES 3EO | 5 | 5 | 5 |
| Sophorolipids | 5 | 5 | 5 |
| Monoethanolamine | 5.95 | 5.95 | 5.95 |
| Trilon M | 2 | 2 | 2 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent 143 | Detergent 144 | Detergent 145 |
|---|---|---|---|
| Na soap from palm | 3.85 | 3.85 | 3.85 |
| kernel oil | | | |
| SFX | 15 | 10 | 20 |
| SLES 3EO | 5 | 5 | 5 |
| REWOTERIC AM C | 5 | 5 | 5 |
| Monoethanolamine | 5.95 | 5.95 | 5.95 |
| Trilon M | 2 | 2 | 2 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent 146 | Detergent 147 | Detergent 148 |
|---|---|---|---|
| Na soap from palm | 3.85 | 3.85 | 3.85 |
| kernel oil | | | |
| SFX | 15 | 10 | 20 |
| SLES 3EO | 5 | 5 | 5 |
| Na-Laurylsulfat | 5 | 5 | 5 |
| (C12-18) | | | |
| Monoethanolamine | 5.95 | 5.95 | 5.95 |
| Trilon M | 2 | 2 | 2 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent 146 | Detergent 147 | Detergent 148 |
|---|---|---|---|
| Na soap from palm | 3.85 | 3.85 | 3.85 |
| kernel oil | | | |
| SFX | 15 | 10 | 20 |
| SLES 3EO | 5 | 5 | 5 |
| C10C16-Alkyl | 5 | 5 | 5 |
| polyglucoside | | | |
| Monoethanolamine | 5.95 | 5.95 | 5.95 |
| Trilon M | 2 | 2 | 2 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent 149 | Detergent 150 | Detergent 151 |
|---|---|---|---|
| Na soap from palm | 3.85 | 3.85 | 3.85 |
| kernel oil | | | |
| SFX | 15 | 10 | 20 |
| SLES 3EO | 5 | 5 | 5 |
| C8C16-Alkyl | 5 | 5 | 5 |
| polyglucoside | | | |
| Monoethanolamine | 5.95 | 5.95 | 5.95 |
| Trilon M | 2 | 2 | 2 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent 149 | Detergent 150 | Detergent 151 |
|---|---|---|---|
| Na soap from palm | 3.85 | 3.85 | 3.85 |
| kernel oil | | | |
| SFX | 15 | 10 | 20 |
| SLES 3EO | 5 | 5 | 5 |
| C8C16-Alkyl | 5 | 5 | 5 |
| polyglucoside | | | |
| Monoethanolamine | 5.95 | 5.95 | 5.95 |
| Trilon M | 2 | 2 | 2 |
| WeylClean PLA 1 | 1 | 1 | 1 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent 152 | Detergent 153 | Detergent 154 |
|---|---|---|---|
| Na soap from palm | 3.85 | 3.85 | 3.85 |
| kernel oil | | | |
| SFX | 15 | 10 | 20 |
| C8C16-Alkyl | 5 | 5 | 5 |
| polyglucoside | | | |
| Monoethanolamine | 5.95 | 5.95 | 5.95 |
| Trilon M | 2 | 2 | 2 |
| Citric acid | 3 | 3 | 3 |
| Medley@ Brilliant | 2 | 2 | 2 |
| 300 L | | | |
| Phenoxyethanol | 1 | 1 | 1 |
| Aluminium chloride | 0.1 | 0.1 | 0.1 |
| | Add water | Add water | Add water |
| | to 100% | to 100% | to 100% |
| pH | 8.0 | 8.2 | 8.1 |

| Ingredients | Detergent 152 | Detergent 153 | Detergent 154 |
|---|---|---|---|
| Na soap from palm | 3.85 | 3.85 | 3.85 |
| kernel oil | | | |
| SFX | 15 | 10 | 20 |
| SLES 3EO | 10 | 10 | 10 |
| C8C16-Alkyl | 5 | 5 | 5 |
| polyglucoside | | | |
| Monoethanolamine | 5.95 | 5.95 | 5.95 |
| Trilon M | 2 | 2 | 2 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent 155 | Detergent 156 | Detergent 157 |
|---|---|---|---|
| Na soap from palm | 3.85 | 3.85 | 3.85 |
| kernel oil | | | |
| SFX | 15 | 10 | 20 |
| SLES 3EO | 5 | 5 | 5 |
| C10C16-Alkyl | 5 | 5 | 5 |
| polyglucoside | | | |
| Monoethanolamine | 5.95 | 5.95 | 5.95 |
| Propylene glycol | 2 | 2 | 2 |
| Trilon M | 2 | 2 | 2 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent 158 | Detergent 159 | Detergent 160 |
|---|---|---|---|
| MEA cocoate | 10 | 10 | 10 |
| SFX | 15 | 10 | 20 |
| SLES 3EO | 10 | 10 | 10 |
| C10C16-Alkyl | 5 | 5 | 5 |
| polyglucoside | | | |
| Monoethanolamine | 5.95 | 5.95 | 5.95 |
| Propylene glycol | 2 | 2 | 2 |
| Trilon M | 2 | 2 | 2 |
| MEA citrate | 5 | 5 | 5 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent 161 | Detergent 162 | Detergent 163 |
|---|---|---|---|
| Na soap from palm | 3.85 | 3.85 | 3.85 |
| kernel oil | | | |
| SFX | 15 | 10 | 20 |
| SLES 3EO | 5 | 5 | 5 |
| C10C16-Alkyl | 5 | 5 | 5 |
| polyglucoside | | | |
| Monoethanolamine | 5.95 | 5.95 | 5.95 |
| Solvents (rthabnols, | 3 | 3 | 3 |
| mono propylene | | | |
| glycol, propylene | | | |
| glycol, dipropylene | | | |
| glycol) | | | |
| Trilon M | 2 | 2 | 2 |
| Citric acid | 3 | 3 | 3 |
| Medley ® Brilliant | 2 | 2 | 2 |
| 300 L | | | |
| Phenoxyethanol | 1 | 1 | 1 |
| | Add water | Add water | Add water |
| | to 100% | to 100% | to 100% |
| pH | 8.0 | 8.2 | 8.1 |

| Ingredients | Detergent 164 | Detergent 165 | Detergent 166 |
|---|---|---|---|
| Na soap from palm | 2 | 2 | 2 |
| kernel oil | | | |
| Sodium Citrate | 6 | 6 | 6 |
| SFX | 15 | 10 | 20 |
| SLES 3EO | 10 | 10 | 10 |
| FAEO C12-18, 7 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant | 2 | 2 | 2 |
| 300 L | | | |
| | Add water | Add water | Add water |
| | to 100% | to 100% | to 100% |
| pH | 8.0 | 8.2 | 8.1 |

| Ingredients | Detergent 167 | Detergent 168 | Detergent 169 |
|---|---|---|---|
| Na soap from palm | 2 | 2 | 2 |
| kernel oil | | | |
| Sodium Citrate | 6 | 6 | 6 |
| SFX | 15 | 0 | 10 |
| SLES 3EO | 10 | 10 | 10 |
| FAEO C12-18, 7 EO | 5 | 5 | 5 |
| Dissolvine ® GLDA | 1 | 1 | 1 |
| Phenoxyethanol | 0.45 | 0.45 | 0.45 |
| NaOH (water solution) | 1 | 1 | 1 |
| Medley ® Brilliant | 2 | 2 | 2 |
| 300 L | | | |
| Manganese sulfate | 1 | 0 | 1 |
| Aluminium chloride | 0 | 0.1 | 0.1 |
| | Add water | Add water | Add water |
| | to 100% | to 100% | to 100% |
| pH | 8.0 | 8.1 | 8.2 |

1. A composition, comprising:
A) 0.5 wt.-% to 90 wt. % of at least one biosurfactant, and
B) 0.01 ppm to 500 ppm manganese ions,
wherein the percentages by weight and the parts per million refer to the total composition.

2. The composition according to claim 1, comprising:
A) 25 wt.-% to 75 wt. % of at least one biosurfactant, and
B) 0.1 ppm to 500 ppm manganese ions,
wherein the percentages by weight and the parts per million refer to the total composition.

3. The composition according to claim 1, comprising:
A) 0.5 wt.-% to 25 wt % of at least one biosurfactant, and
B) 0.01 ppm to 500 ppm manganese ions,
wherein the percentages by weight and the parts per million refer to the total composition.

4. The composition according to claim 1, wherein said at least one biosurfactant is selected from the group consisting of rhamnolipids, glucolipids and sophorolipids.

5. The composition according to claim 1, further comprising:
C) at least one preservative.

6. The composition according to claim 5, wherein said at least one preservative is selected from the group consisting of p-anisic acid, benzoic acid, levulinic acid, sorbic acid, lactic acid, mandelic acid, salicylic acid, dehydroacetic acid, caprylhydroxamic acid, cinnamic acid, geranic acid, pelargonic acid, and the salts of the aforementioned acids, methylparaben, ethylparaben, phenylpropanol, benzyl alcohol, phenethyl alcohol, phenoxyethanol, propanediol, pentylene glycol, 1,2-hexanediol, caprylyl glycol, undecyl alcohol, methylpropanediol, ethylhexylglycerin, n-octylglycerin, n-heptylglycerin, n-hexylglycerin, methylheptylglycerin, undecylenamidopropyltrimonium methosulfate, isothiazolinones, triethyl citrate, citral, hydroxymethylglycinate, glyceryl caprylate, iodopropynyl butylcarbamat and Sodium Caproyl/Lauroyl Lactylate.

7. A method of stabilizing the colour of a composition comprising a biosurfactant, the method comprising:
a) providing a composition comprising a biosurfactant, and
b) adding manganese to the composition to a concentration of 0.01 ppm to 500 ppm manganese ions, wherein the parts per million refer to the total composition.

8. The method according to claim 7, wherein the manganese is added in the form of manganese oxide and/or a manganese salt.

9. The method according to claim 7, wherein said at least one biosurfactant is selected from the group consisting of rhamnolipids, glucolipids and sophorolipids.

10. The method according to claim 7, wherein said composition comprising a biosurfactant provided in a) has a Gardner colour value in a range of from 0.01 to 15.

11. The method according to claim 7, wherein said composition comprising a biosurfactant provided in a) has an L colour value of the CIELAB colour space in a range of from 20 to 100.

12. A method, comprising:
improving the colour stability of a composition comprising at least one biosurfactant with manganese ions.

13. A method, comprising:
preventing malodour of a composition comprising at least one biosurfactant with manganese ions.
